(19) United States
(12) Patent Application Publication
(10) Pub. No.: US 2024/0108718 A1
(43) Pub. Date: Apr. 4, 2024

(54) NUCLEIC ACID VACCINES

(71) Applicants: University of Copenhagen, Copenhagen K (DK); AdaptVac ApS, Copenhagen N (DK)

(72) Inventors: Adam Frederik Sander BERTELSEN, Dragør (DK); Morten Agertoug NIELSEN, Birkerød (DK); Louise GOKSØYR, Copenhagen N (DK); Cyrielle Elyette FOUGEROUX, Copenhagen S (DK); Willem Adriaan DE JONGH, Manchester (GB)

(21) Appl. No.: 18/257,554

(22) PCT Filed: Dec. 17, 2021
(86) PCT No.: PCT/EP2021/086528

§ 371 Date: Jun. 14, 2023

(30) Foreign Application Priority Data
Dec. 18, 2020 (EP) 20215653.5
Mar. 9, 2021 (EP) 21161436.7

(51) Int. Cl. A61K 39/29

(52) U.S. Cl. CPC A61K 39/292; A61K 2039/53

(57) ABSTRACT

The present invention relates to modular nanoparticle-based compositions based on nucleic acids, such as DNA and RNA, which are particularly useful in prophylaxis and/or treatment of diseases and disorders.

### TECHNICAL FIELD

The present invention relates to modular nanoparticle-based compositions based on nucleic acids, such as DNA and RNA, which are particularly useful in prophylaxis and/or treatment of diseases and disorders.

### BACKGROUND

Vaccines remain the most effective tools for preventing and controlling the spread of infectious diseases. Live-attenuated vaccines are highly immunogenic, inducing long-lived antibody responses even after a single immunization. In contrast, modern, subunit vaccines (i.e. based on a soluble protein antigen) show high safety, but reduced immunogenicity and fail to induce similar durable antibody responses in humans. Induction of a strong and long lasting immune response to pathogens as well as disease-associated antigens is thus very difficult to obtain with simple subunit vaccines.

The licensed Human papillomavirus (HPV) vaccines (Cervarix®, Gardasil®, and Gardasil 9®), however, make an important exception, since they seem to have comparable immunogenicity to live-attenuated vaccines and can induce highly potent, durable antibody responses in humans, even after a single dose (Schiller et al., 2018, Schiller et al., 2012, De Vincenzo et al., 2014). Importantly, this vaccine is formed by the self-assembly of the HPV major capsid protein into virus-like particles (VLPs), which is believed to be the cause of its high potency.

In fact, many studies have established a strong causal link between the high immunogenicity of VLPs and their structural similarities to native viruses, and several strategies have been pursued, exploiting VLPs as scaffolds for the presentation of heterologous antigens, including self-antigens. These studies have collectively shown that multivalent, repetitive antigen display can, in fact, significantly increase the immunogenicity of an antigen and even induce long-lasting immunity.

We have previously described the development of a modular VLP-based vaccine platform using a split-protein (Tag/Catcher) conjugation technology for attaching antigens in a unidirectional, multivalent and repetitive manner on the surface of VLPs (WO 2016/112921). To date, this technology represents a highly powerful and versatile vaccine platform.

DNA- and/or RNA-based vaccines, in contrast to protein-based vaccines, possess major advantages, mainly due to their simplicity and low cost of production; omitting the steps of recombinant expression and purification of the vaccine antigen. In fact, usually clinical vaccine batches can already be generated shortly after a sequence encoding the antigen has become available. A further advantage is that the manufacturing process is cell-free and highly scalable. Additionally, the manufacturing of multiple different vaccines at a facility requires minimal adaptation of the manufacturing processes to the specific vaccine formulations. Finally, in vivo expression of complex proteins that are difficult or impossible to generate recombinantly with current expression systems is possible with these vaccines.

However, so far DNA vaccines have been shown to induce only relatively weak immune responses against the antigen when tested in humans and non-human primates, limiting their commercial exploitation.

There is thus an urgent need for a vaccine that combines the high immunogenicity of multivalent particulate vaccines with the manufacturing advantages associated with nucleic acid based vaccines.

### SUMMARY

The present invention provides nucleic acid based vaccines, which upon delivery into eukaryotic cells during vaccination, are translated into self-assembling nanoparticles displaying the vaccine antigen in a unidirectional, repetitive and multivalent manner by exploiting a split-protein Tag/Catcher conjugation system.

The repetitive, multivalent antigen display increases the immunogenicity of the vaccine antigen, enabling induction of a strong antigen-specific immune response after vaccination. At the same time, the nucleic acid (DNA and/or mRNA) based vaccine technology holds major benefits in terms of manufacturing, as the up- and down-stream processes associated with recombinant production of the vaccine antigen can be omitted. Additionally, delivery of the vaccine antigen as a nucleic acid sequence allows in vivo translation of the encoded proteins, which may be otherwise difficult or impossible to produce recombinantly.

Herein is provided a composition comprising:
i. a first polynucleotide encoding a protein fused to a first peptide tag; and
ii. a second polynucleotide encoding an antigen fused to a second peptide tag,
wherein the antigen and the protein upon expression in a cell are linked via an isopeptide bond, or an ester bond, between the first peptide tag and the second peptide tag, whereby i-ii form a particle displaying said antigen.

Herein is also provided a composition as disclosed herein for use in the prophylaxis and/or treatment of a disease in a subject in need thereof.

Herein is also provided a method of preventing or treating a disease in a subject in need thereof.

Herein is also provided an expression system comprising:
i. a first polynucleotide encoding a protein fused to a first peptide tag; and
ii. a second polynucleotide encoding an antigen fused to a second peptide tag,
   wherein upon expression of the first and second polynucleotides in a cell, the antigen and the protein are linked via an isopeptide bond, or an ester bond, between the first peptide tag and the second peptide tag, whereby i-ii form a particle displaying said antigen.

Herein is also provided a cell expressing:
i. a first polynucleotide encoding a protein fused to a first peptide tag, preferably as defined in any one of the preceding claims; and
ii. a second polynucleotide encoding an antigen fused to a second peptide tag, preferably as defined in any one of the preceding claims,
   wherein the antigen and the protein upon expression in a cell are linked via an isopeptide bond, or an ester bond, between the first peptide tag and the second peptide tag, whereby i-ii form a particle displaying said antigen.

Herein is also provided a host cell comprising an expression system as disclosed herein.

Herein is also provided a method of administering a composition for use in the prophylaxis and/or treatment of a disease in a subject in need thereof comprising the steps of
i. obtaining at least one composition as disclosed herein, and
ii. administering said composition to a subject at least once for prophylaxis and/or treatment of a disease as defined herein.

Herein is also provided a kit of parts comprising
i. a composition or an expression system as defined herein, and
ii. optionally, a medical instrument or other means for administering the composition, and
iii. instructions for use.

### DESCRIPTION OF THE DRAWINGS

FIG. 1: Confocal laser scanning microscopy (CLSM) of C2C12 mouse cells 72 hours after co-transfection with SpyCatcher (SpyC)-eGFP and Spytagged (SpytT) HBcore. The picture shows distinct particulate enhanced green fluorescent protein (eGFP) fluorescent signals with a peri-nuclear distribution (all panels).

FIG. 2: Confocal laser scanning microscopy (CLSM) of C2C12 mouse cells 72 hours after co-transfected with SpyCatcher (SpyC)-eGFP and Spytagged (SpytT) AP205 coat protein. The picture shows an even green smear from the eGFP fluorescence throughout the cell (all panels). Scale bar shows 50 μm.

FIG. 3: Western blot (WB) probed with polyclonal anti-eGFP antibodies for detection of eGFP in pre (UC input) and post ultracentrifugation (UC) fractions (3-21 and 31) of sonicated C2C12 cell co-transfected with Spytagged (SpytT) AP205 coat protein and SpyCatcher (SpyC)-eGFP. The theoretical size of eGFP is 41 kDa whereas the conjugation of eGFP and AP205 would be 59 kDa. A band of approximately 59 kDa is seen weekly in the eGFP and more strongly in the UC input sample, but not in any of the post UC fractionated samples i.e. there is no indication that AP205 particles have formed to which eGFP have bound.

FIG. 4: Western blot (WB) probed with polyclonal anti-eGFP antibodies for detection of eGFP in pre (UC input) and post ultracentrifugation (UC) fractions (3-21 and 31) of sonicated C2C12 cell co-transfected with Spytagged (SpyC) HBcore antigen and SpyCatcher (SpyC)-eGFP. The theoretical size of eGFP is 41 kDa whereas the conjugation of eGFP and HBcore antigen is 64 kDa. A band of approximately 64 kDa is seen in the UC-input sample as well as in post-UC fractions, indicating that particulate structures have formed from the conjugated SpyTHBc and SpyC eGFP proteins.

FIG. 5: Vector map of pVAX1 (V26020, thermoFisher). CMV promoter: bases 137-724; T7 promoter/priming site: bases 664-683; multiple cloning site: bases 696-811; bovine growth hormone (BGH) reverse priming site: bases 823-840; BGH polyadenylation signal: bases 829-1053; kanamycin resistance gene: bases 1226-2020; pUC origin: bases 2320-2993.

FIG. 6: Expression of particle-forming subunit proteins from plasmid DNA. Western blot (WB) images of particle-forming subunits after plasmid DNA transfection in HEK cells. HEK cells were transfected with plasmid DNA encoding for particle forming subunit. Cells and supernatant were harvested 6 days after transfection and ran on a WB. Relevant antibodies coupled to horseradish peroxidase (HRP) were used for detection of the particle forming subunit.
A. Sample: sign3-SpyC-i301-ctag. Primary Ab: aSpyC (mouse sera). Secondary Ab: anti-mouse-HRP. Expected size: 35.7 kDa.
B. Sample: sign8-tandemHBc-SpyC. Primary Ab: aHBc (mouse sera). Secondary Ab: anti-mouse-HRP. Expected size: 52.3 kDa.
C. Sample: sign8-tandemHBc-SpyT. Primary Ab: aHBc (mouse sera). Secondary Ab: anti-mouse-HRP. Expected size: 23.12 kDa.
D. Sample: sign9-Ferritin-SpyC. Primary Ab: aSpyC (mouse sera). Secondary Ab: anti-mouse-HRP. Expected size: 32.6 kDa.

These pictures show band of the expected size in cell and supernatant samples for all constructs, thus indicating expression and secretion of the particle-forming subunit proteins in HEK cells, after plasmid DNA transfection.

FIG. 7: Expression of soluble proteins from plasmid DNA. Western blot images of soluble proteins after plasmid DNA transfection in HEK cells. HEK cells were transfected with plasmid DNA encoding for soluble protein. Cells and supernatant were harvested 6 days after transfection and ran on a WB. Relevant antibodies coupled to HRP were used for detection of the soluble protein.
A. Sample: sign8-SpyT-eGFP. Primary Ab: aGFP-HRP. Expected size: 30.8 kDa.
B. Sample: sign8-eGFP. Primary Ab: aGFP-HRP. Expected size: 29 kDa.
C. Sample: sign8-SpyC-His. Primary Ab: aSpyC (mouse sera). Secondary Ab: anti-mouse-HRP. Expected size: 15.4 kDa.
D. Sample: sign7-Pfs25-SpyT-Ctag. Primary Ab: aCtag-biotin. Secondary Ab: strep-HRP. Expected size: 24.3 kDa.
E. Sample: sign8-SpyC-eGFP. Primary Ab: aGFP-HRP. Expected size: 41.7 kDa.

These pictures show bands of the expected size in cell and supernatant for all constructs, thus indicating expression and secretion of soluble proteins in HEK cells, after plasmid DNA transfection.

FIG. 8: Verification of the conjugation of eGFP to different particle-forming proteins. Western blot (WB) images of coupled eGFP to particle forming proteins after plasmid DNA co-transfection in HEK cells. HEK cells were co-transfected with plasmid DNA encoding for eGFP+tag/catcher and plasmid DNA encoding for particle-forming proteins+corresponding tag/catcher. Cells and supernatant were harvested 6 days after co-transfection. Relevant antibodies coupled to HRP were used for detection of the coupled GFP to the particle forming subunit.
A. Sample: sign8-SpyT-eGFP and sign9-SpyC-Ferritin. Primary Ab: aGFP-HRP. Expected size: 62 kDa.
B. Sample: sign8-SpyC-eGFP and sign8-Hbc-SpyT. Primary Ab: aHBc (mouse serum). Secondary Ab: anti-mouse-HRP. Expected size: 64.8 kDa.
C. Sample: sign8-tandemHBc-SpyC and sign8-SpyT-eGFP. Primary Ab: aGFP-HRP. Expected size: 83.1 kDa.
D. Sample: sign3-SpyC-i301-ctag and sign8-SpyT-eGFP. Primary Ab: aGFP-HRP. Expected size: 66.5 kDa.
E. Sample: sign9-SpyT-E2 and sign8-SpyC-eGFP. Primary Ab: aGFP-HRP. Expected size: 72.45 kDa.
F. Sample: sign9-SpyT-LS and sign8-SpyC-eGFP. Primary Ab: aGFP-HRP. Expected size: 61.7 kDa.

These pictures show bands of the expected size for coupled eGFP to different particle-forming subunit proteins in cells and supernatant for all constructs. This indicates that eGFP and the particles with the corresponding tag/catcher are able to couple in vitro, after co-transfection in HEK cells.

FIG. 9: Verification of the conjugation of SpyC to different particle-forming proteins. Western blot (WB) images of coupled SpyC to particle forming proteins after plasmid DNA co-transfection in HEK cells. HEK cells were co-transfected with plasmid DNA encoding for SpyC and plasmid DNA encoding for particle-forming proteins+corresponding tag. Cells and supernatant were harvested 6 days after co-transfection. Relevant antibodies coupled to HRP were used for detection of the coupled SpyC to the particle forming subunits.
A. Sample: sign8-SpyC-His and sign9-SpyT-E2. Primary Ab: aHis-HRP. Expected size: 46.15 kDa.
B. Sample: sign8-SpyC-His and sign9-LS-SpyT. Primary Ab: aHis-HRP. Expected size: 35.4 kDa.
C. Sample: sign8-SpyC-His and sign8-Hbc-SpyT. Primary Ab: aHis-HRP. Expected size: 38.5 kDa.
D. Sample: sign8-Norovirus-SpyT and sign8-SpyC-His. Primary Ab: aHis-HRP. Expected size: 78.16 kDa.

These pictures show bands of the expected size for coupled SpyC to different particle-forming subunit proteins in cells and supernatant for all constructs. This indicates that SpyC and the particle with the corresponding tag/catcher are able to couple in vitro, after co-transfection in HEK cells.

FIG. 10: Verification of the conjugation of Pfs25 to different particle-forming proteins. Western blot (WB) images of coupled Pfs25 to particle forming proteins after plasmid DNA co-transfection in HEK cells. HEK cells were co-transfected with plasmid DNA encoding for Pfs25-SpyT and plasmid DNA encoding for particle-forming proteins+corresponding catcher. Cells and supernatant were harvested 6 days after co-transfection. Relevant antibodies coupled to HRP were used for detection of the coupled Pfs25 to the particle forming subunit.
A. Sample: sign9-SpyC-Ferritin and sign7-Pfs25-SpyT-Ctag. Primary Ab: aCtag-biotin. Secondary Ab: strep-HRP. Expected size: 56.9a.
B. Sample: sign3-SpyC-i301-Ctag and sign7-Pfs25-SpyT-Ctag. Primary Ab: aCtag-biotin. Secondary Ab: strep-HRP. Expected size: 60 kDa.

These pictures show bands of the expected size for coupled Pfs25 to different particle-forming subunit proteins in cell and supernatant for all constructs. This indicates that Pfs25 and the particle with the corresponding tag/catcher are able to couple in vitro, after co-transfection in HEK cells.

FIG. 11: Verifying nanoparticle formation by ultracentrifugation (UC) and WB. Western blot (WB) image of UC fractions from HEK cells transfected supernatant. HEK cells were transfected with plasmid DNA encoding for particle-forming subunit proteins. Supernatant was harvested 6 days after transfection and loaded on an optiprep density gradient. The gradient was spun down for 3 h30, 47800 RPM, 16° C., and fractionated into 12 fractions. Each fraction was run on a WB where relevant antibodies coupled to HRP were used for detection of the particle forming subunit. If any particles are formed, bands of the corresponding size will be expected in fractions 3-8.
A. Sample: sign3-SpyC-i301-Ctag. Primary Ab: aSpyC (mouse sera). Secondary Ab: anti-mouse-HRP. Expected size 36 kDa.
B. Sample: sign8-tandemHBc-SpyCatcher. Primary Ab: aHBc (mouse sera). Secondary Ab: anti-mouse-HRP. Expected size 52 kDa.
C. Sample: sign9-Ferritin-SpyC. Primary Ab: aSpyC (mouse sera). Secondary Ab: anti-mouse-HRP. Expected size 32.6 kDa.

These pictures show particle formation in all constructs, as we can see bands of the expected size present in relevant fractions (3-8). Thus, this indicates that after transfection with plasmid DNA in HEK cells, there was not only expression and secretion of the particle subunit, but also formation of a particle in vitro.

FIG. 12: Verifying coupled nanoparticle formation by UC and Western blot (WB). WB image of UC fractions from HEK cells transfected supernatant. HEK cells were co-transfected with plasmid DNA encoding for soluble antigen+tag/catcher and plasmid DNA encoding for particle-forming proteins+corresponding tag/catcher. Supernatant was harvested 6 days after transfection and loaded on an optiprep density gradient. The gradient was spun down for 3 h30, 47800 RPM, 16° C., and fractionated into 12 fractions. Each fraction was run on a WB where relevant antibodies coupled to HRP were used for detection of the coupled antigen to the particle forming subunit. If any particles are formed, bands of the corresponding size will be expected in fractions 3-8.
A. Sample: sign8-SpyT-eGFP+sign9-SpyC-Ferritin. Primary Ab: aGFP-HRP. Expected size 62 kDa.
B. Sample: sign8-SpyC-His+sign9-SpyT-E2. Primary Ab: aHis-HRP. Expected size 46.15 kDa.
C. Sample: sign8-SpyC-His+sign9-LS-SpyT. Primary Ab: aHis-HRP. Expected size 35.4 kDa.
D. Sample: sign9-SpyC-Ferritin+sign7-Pfs25-SpyT. Primary Ab: aCtag-biotin. Secondary Ab: strep-HRP. Expected size 56.9 kDa.
E. Sample: sign3-SpyC-i301+sign7-Pfs25-SpyT. Primary Ab: aCtag-biotin. Secondary Ab: strep-HRP. Expected size 60 kDa.
F. Sample: sign9-SpyT-E2+sign8-SpyC-eGFP. Primary Ab: aGFP-HRP. Expected size 72.45 kDa.
G. Sample: sign9-LS-SpyT+sign8-SpyC-eGFP. Primary Ab: aGFP-HRP. Expected size 61.7 kDa.

These pictures show coupled particle formation in all constructs, as we can see bands of the expected size present in relevant fractions (3-8). Thus, this indicates that after co-transfection with plasmid DNA in HEK cells, there was not only expression and secretion of the particle subunit and the soluble antigens, but also formation of a particle coupled to an antigen in vitro.

FIG. 13: Visualisation of particle formation by transmission electronic microscopy (TEM). Electron microscopy images of UC purified supernatant from HEK cells transfected with plasmid DNA. HEK cells were transfected with plasmid DNA encoding for particle forming subunit or co-transfected with plasmid DNA encoding for soluble antigen+tag/catcher and plasmid DNA encoding for particle-forming proteins+corresponding tag/catcher. Supernatant was harvested 6 days after transfection and loaded on an optiprep density gradient. The gradient was spun down for 3 h30, 47800 RPM, 16° C., and fractionated into 12 fractions. The fractions containing particle were pooled and dialysis into 1×PBS for TEM imaging.
A. Sample: sign9-Ferritin-SpyC. Expected size: 12 nm.
B. Sample: sign8-SpyC-His+sign9-LS-SpyT. Expected size: 12 nm.
C. Sample: sign9-SpyT-E2+sign8-SpyC-His. Expected size: 12 nm.
D. Sample: sign8-SpyT-eGFP+sign9-SpyC-Ferritin. Expected size: 12 nm
E. Sample: sign3-SpyC-i301-Ctag+sign7-Pfs25-SpyT-Ctag. Expected size: 25 nm.
F. Sample: sign9-SpyC-Ferritin+sign7-Pfs25-SpyT-Ctag. Expected size: 12 nm.

These pictures show particles of the expected size formed in the SN of transfected or co-transfected cells. Thus, we can further confirm that particles and coupled particles are able to be formed in the supernatant of transfected or co-transfected cells in vitro from plasmid DNA.

FIG. 14: Verification that conjugation of the antigen to the nanoparticle-forming protein occurred intracellularly. Western blot (WB) images of coupled soluble antigen to particle forming proteins after plasmid DNA co-transfection in HEK cells. HEK cells were co-transfected with plasmid DNA encoding for a soluble antigen+tag/catcher and plasmid DNA encoding for particle-forming proteins+corresponding tag/catcher. Cells and supernatant were harvested 6 days after co-transfection. Upon harvest, cells were resuspended in 1×SDS+DTT, to prevent further coupling. Cell samples in 1×SDS+DTT and supernatant were run on WB. Relevant antibodies coupled to HRP were used for detection of the coupled soluble antigen to the particle forming subunit. Thus if any coupling is seen on WB, it had to occur intracellularly before harvest.
A. Sample: sign8-SpyC-His and sign9-LS-SpyT. Primary Ab: aSpyC (mouse sera). Secondary Ab: anti-mouse-HRP. Expected size: 35.4 kDa.
B. Sample: sign8-SpyT-eGFP+sign9-SpyC-Ferritin (B1822+B1772). Primary Ab: aSpyC (mouse sera). Secondary Ab: anti-mouse-HRP. Expected size: 63.4 kDa.
C. Sample: sign3-SpyC-i301-ctag (B1774) and sign8-SpyT-eGFP (B1822). Primary Ab: aHis-HRP. Expected size: 66.5 kDa.
D. Sample: sign8-eGFP-SpyC (B2009) and sign9-LS-SpyT (B1929) Lane 1 (cell), 2 (SN) compared to sign8-eGFP (B2033) and sign9-LS-SpyT (B1929) Lane 3 (cell), 4 (SN). Primary Ab: aGFP-HRP. Expected: B2009+B1929 should be able to couple while B2033+B1929 should not (since there are not catcher on B2033).

These pictures show that particles and soluble antigens are able to couple intracellularly, and not only in the supernatant. Indeed, a band was visualised of the expected coupling size in cells harvested with SDS+DTT, thus indicating that the coupling occurred inside the cells in the in vitro culture.

FIG. 15: Verification of the immunogenicity of the plasmid DNA vaccine encoding for particle forming subunit. ELISA titers of total IgG in mice after plasmid DNA immunization. Plasmid DNA encoding for particles and/or soluble antigen was used for vaccination in mice. Balb/c mice were immunized with 30 ug of LS-SpyT (B1929) and 30 ug of SpyC (B1928) (N=6), or 30 ug of SpyC (B1928) (N=4), or 30 ug of E2-SpyT (B1930) and 30 ug of SpyC (B1928). DNA was formulated in PBS and injected in the right thigh muscle. Mice were immunized on day 0 and week 5, and blood was drawn on week 3 and 4 post prime and post boost. Serum was isolated from the blood and run on ELISA for detection of anti-SpyC IgG. For that purpose, 96-well plates (Nunc MaxiSorp) were coated SpyC in PBS. IgG against SpyC were detected with a HRP conjugated goat anti-mouse IgG (Life technologies, A16072). Plates were developed with TMB X-tra substrate (Kem-En-Tec, 4800A) and absorbance was measured at 450 nM.
A. ELISA titers against SpyC 3 weeks and 4 weeks post prime immunization in Balb/c mice
B. ELISA titers against SpyC 3 weeks and 4 weeks post boost immunization in Balb/c mice

These ELISA titers show that mice receiving DNA encoding for the particles and DNA encoding for SpyC, have higher IgG titers against SpyC after a first immunization, compared to mice receiving only DNA encoding for SpyC. Additionally, this trend is even higher after a boost immunization. This is true for both groups of mice that have received SpyC in combination with either the LS particle or the E2 particle.

### DETAILED DESCRIPTION OF THE INVENTION

The present disclosure provides nucleic acid based vaccines, which upon delivery into eukaryotic cells during vaccination, are translated into self-assembling nanoparticles displaying the vaccine antigen in a unidirectional, repetitive and multivalent manner by exploiting a split-protein Tag/Catcher conjugation system.

The repetitive, multivalent antigen display increases the immunogenicity of the vaccine antigen, enabling induction of a strong antigen-specific immune response after vaccination. At the same time, the nucleic acid (DNA and/or mRNA) based vaccine technology holds major benefits in terms of manufacturing, as the up- and down-stream processes associated with recombinant production of the vaccine antigen can be omitted. In addition, delivery of the vaccine antigen as a nucleic acid sequence allows in vivo translation of the encoded proteins, which may be otherwise difficult or impossible to produce recombinantly.

The solution of the present invention represents a novel approach for making a versatile nucleic-acid based vaccine delivery platform capable of efficiently displaying antigen epitopes and of inducing long-term protective immunity.

### Definitions

The term “isopeptide bond” as used herein, refers to an amide bond between a carboxyl group and an amino group at least one of which is not derived from a protein main chain or alternatively viewed is not part of the protein backbone. An isopeptide bond may form within a single protein or may occur between two peptides or a peptide and a protein. Thus, an isopeptide may form intramolecularly within a single protein or intermolecularly i.e. between two peptide/protein molecules. Typically, an isopeptide bond may occur intramolecularly between two reactive amino acids: a lysine and an asparagine or aspartate. For the process to occur the two reactive amino acids need to be in close proximity in a hydrophobic environment often including aromatic residues. Finally, the autocatalytic process may be facilitated by a catalytic aspartate or glutamate residue, which do not themselves take part in the isopeptide bond.

In the case of intermolecular isopeptide bonds, the bond typically occurs between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. Thus, an isopeptide bond may form between a lysine residue and an asparagine residue or between a lysine residue and an aspartic acid residue. Particularly, isopeptide bonds can occur between the side chain amine of lysine and carboxamide group of asparagine.

The term “open reading frame” as used herein refers to a nucleotide sequence comprising in a 5′ to 3′ direction 1) a translation initiation codon, 2) one or more codons coding for one or more gene products of interest, preferably one or more protein, and 3) a translation stop codon, whereby it is understood that 1), 2) and 3) are operably linked in frame. The open reading frame will thus consist of a multiple of 3 nucleotides (triplets).

The term “sequence variant” refers to a polypeptide and/or polynucleotide sequence with at least 70%, such as 75%, such as 80%, such as 85%, such as 90%, such as 95%, such as 96%, such as, 97%, such as 98%, such as 99%, such as 99.5%, such as 100% sequence identity to said polypeptide and/or polynucleotide sequence.

The term “antigenic variant” refers to a variant of the full length of a polypeptide or a fragment of said polypeptide, wherein the fragment comprises an epitope that is recognized by a cytotoxic T lymphocyte, helper T lymphocyte and/or B cell of the host. Said fragment may be more immunogenic and thus elicit a stronger and/or longer lasting immune response than the original polypeptide from which it is derived. Preferably, the immunogenic portion of the antigenic variant will comprise at least 30%, preferably at least 50%, especially at least 75% and in particular at least 90% (e.g. 95% or 98%) of the amino acid sequence of the reference sequence. The immunogenic portion will preferably comprise all of the epitope regions of the reference sequence. The immunogenicity of said antigenic variant may be verified by any of the known methods in the art, such as the methods described in Wadhwa et al., 2015.

A first peptide tag and a second peptide tag (or binding partner) as discussed herein refer to a first and second peptide tag which bind to one another via an isopeptide bond, preferably a spontaneous isopeptide bond. Preferably the first peptide tag comprises one of the reactive residues involved in the isopeptide bond and the second peptide tag comprises the other reactive residue involved in that isopeptide bond.

The term “spontaneous” as used herein refers to a bond, in particular an isopeptide bond, which can form in a protein or between peptides or proteins (e.g. between the first peptide tag and the second peptide tag) without any other agent (e.g. an enzyme catalyst) being present and/or without chemical modification of the protein or peptide e.g. without native chemical ligation or chemical coupling. A spontaneous isopeptide bond may therefore form of its own accord in the absence of enzymes or other exogenous substances or without chemical modification. Particularly however, a spontaneous isopeptide or covalent bond may require the presence of a glutamic acid or an aspartic acid residue in one of the peptides/proteins involved in the bond to allow formation of the bond.

The term “virus-like particle” or “VLP” refers to one or several recombinantly expressed viral proteins such as viral capsid proteins, which spontaneously assemble into macromolecular particulate structures mimicking the morphology of a virus coat, but lacking infectious genetic material.

The term “particle” herein refers to a virus-like particle or to a nanoparticle, on the surface of which an antigen can be displayed as described herein. The surface may be an internal surface, i.e. facing towards the inner part of the particle, or an external surface, i.e. facing towards the surroundings of the particle.

The term “self-assembly” refers to a process in which a system of pre-existing components, under specific conditions, adopts a more organised structure through interactions between the components themselves. In the present context, self-assembly refers to the intrinsic capacity of a protein, such as a viral protein, for example a capsid protein, and/or a phage protein to self-assemble into particles, in particular virus-like particles in the absence of other viral proteins, when subjected to specific conditions. “Self-assembly” does not preclude the possibility that cellular proteins, e.g. chaperones, participate in the process of intracellular VLP or nanoparticle assembly. The self-assembly process may be sensitive and fragile and may be influenced by factors such as, but not limited to, choice of expression host, choice of expression conditions, and conditions for maturing the virus-like particles. Virus capsid proteins may be able to form VLPs on their own, or in combination with several virus capsid proteins, these optionally all being identical.

The term “consistent orientation”, as used herein, refers to the orientation of an antigen and its spatial orientation on the surface of a particle as disclosed herein, i.e. on an internal surface or on an external surface of the particle, preferably at least on the external surface. When linking an antigen fused to a second peptide tag to protein comprising a second peptide tag as disclosed herein, one molecule of antigen can only be linked to a single particle-forming protein at unique sites in both the antigen and the protein, thus creating a uniform and/or consistent presentation of said antigen with a consistent orientation. In contrast, for example, a streptavidin homo-tetramer may crosslink several proteins on the outer surface of a biotinylated VLP, thus creating an irregular and non-consistent orientation of said antigen. Besides, it is highly challenging to use streptavidin as a bridging molecule e.g. for conjugating biotinylated antigens onto biotinylated VLPs, since the multiple biotin binding sites will allow cross-linking and aggregation of the biotinylated VLPs.

The term “regularly spaced” as used herein, refers to antigens of the present invention which forms a pattern on a surface of a VLP or nanoparticle. Such pattern may be symmetric, circle-like, and/or bouquet like pattern of antigens.

The term “treatment” refers to the remediation of a health problem. Treatment may also be preventive and/or prophylactic or reduce the risk of the occurrence of a disease and/or infection. Treatment may also be curative or ameliorate a disease and/or infection.

The term “prophylaxis” refers to the reduction of risk of the occurrence of a disease and/or infection. Prophylaxis may also refer to the prevention of the occurrence of a disease and/or infection.

The term “loop” refers to a secondary structure of a polypeptide where the polypeptide chain reverses its overall direction and may also be referred to as a turn.

The term “vaccine cocktail” refers to a mixture of antigens administered together. A vaccine cocktail may be administered as a single dose or as several doses administered over a period of time. Time intervals may be, but are not limited to, administration within the same year, month, week, day, hour and/or minute. Co-vaccination and vaccine cocktail may be used interchangeably.

The term “self-antigens” refers to endogenous antigens that have been generated within previously normal cells as a result of abnormal cell metabolism.

### Compositions

Herein is provided a composition comprising:
i. a first polynucleotide encoding a protein fused to a first peptide tag; and
ii. a second polynucleotide encoding an antigen fused to a second peptide tag,
wherein the antigen and the protein upon expression in a cell are linked via an isopeptide bond, or an ester bond, between the first peptide tag and the second peptide tag, whereby i-ii form a particle displaying said antigen.

Said compositions are useful for prophylaxis and/or treatment of a disease or a disorder, such as those described herein below.

Administration of said composition comprising said first and second polynucleotides in a subject may induce a stronger immune response in said subject compared to administration of a composition comprising the polypeptides encoded by said first and second polynucleotides, wherein said first and second polypeptide have been linked via an isopeptide bond, or via an ester bond, between the first peptide tag and the second peptide tag prior to administration in the subject.

The first peptide tag and the second peptide tag are selected from peptides having the intrinsic ability to form an isopeptide bond, or an ester bond, thereby binding to one another. The first polynucleotide encodes a protein which has the ability to form, preferably spontaneously, a particle, such as a nanoparticle or a virus-like particle (VLP). Upon expression in a cell, the first polynucleotide thus leads to the formation of particles formed by the protein fused to the first peptide tag, while the second polynucleotide leads to expression of a fusion protein comprising or consisting of an antigen fused to the second peptide tag. The assembled particles may closely resemble viruses or other pathogenic organisms that are recognized by the immune system, but are non-infectious because they contain no pathogenic genetic material. Due to the spontaneous formation of an isopeptide bond, or an ester bond, between the first peptide tag and the second peptide tag, particles are formed, which display the antigen on their surface, preferably on their external surface. Importantly, besides consistent antigen orientation, such nanoparticles and VLPs unexpectedly have unusually beneficial antigen display characteristics including consistent antigen orientation, high-density, and regular spacing. The obtained nanoparticles and VLPs can thus induce strong humoral responses and overcome B cell tolerance. The successful formation of such nanoparticles or VLPs may be assessed by relevant methods as known to the person skilled in the art, such as those disclosed in Example 2 and 3 of the present disclosure. Such nanoparticles or VLPs surprisingly show an increased efficiency of antigen coupling compared to chemical coupling methods, and allow antigen display with consistent antigen orientation, high-density, and regular spacing, for both large and small antigens.

The first and second polynucleotides may be DNA or RNA; preferably, the first polynucleotide and the second polynucleotides are both DNA, or both RNA.

The RNA constructs and/or the mRNA transcribed from the DNA constructs may be polycistronic. In some embodiments, the first and the second polynucleotides are encoded on the same ribonucleic acid molecule. In some embodiments, the first and the second polynucleotides lie within the same open reading frame, whereby only one promoter sequence is needed to transcribe both polynucleotides. In some embodiments, the first and the second polynucleotides lie within separate open reading frames and may thus be regulated by separate promoters.

### Proteins

In preferred embodiments, the protein is a particle-forming protein. For example, the protein may be a viral capsid protein or a viral envelope protein such as a glycoprotein. In some embodiments, the protein is from a mammalian virus, for example a human virus.

In some embodiments, the protein is a protein from a hepatitis virus such as hepatitis B or E, for example a core protein from hepatitis B virus. In some embodiments, the protein is a protein from a norovirus such as NoV. In some embodiments, the protein is a protein from a papilloma virus such as Human Papilloma Virus (HPV), preferably HPV16 or HPV18, such as HPV L1. In some embodiments, the protein a protein from a polyomavirus such as polyomavirus vp1 (PyV). In some embodiments, the protein is a protein from a calicivirus such as feline calicivirus (FCV), preferably FCV VP1. In some embodiments, the protein is a protein from a circovirus such as a porcine circovirus (PCV), preferably PCV2 ORF2. In some embodiments, the protein is a protein from a nervous necrosis virus (NNV), such as NNV coat protein. In some embodiments, the protein is a protein from a parvovirus such as canine parvovirus (CVP), preferably CPV VP2, goose parvovirus (GPV) or porcine parvovirus (PPV), preferably structural proteins from GPV or PPV, or parvovirus B19. In some embodiments, the protein is a protein from a protoparvovirus such as an enteritis virus, for example mink enteritis virus (MEV), preferably MEV VP2, or duck plague virus (DPV), preferably a DPV structural protein.

The protein may be a protein from a plant virus, such as a cowpea virus, a tobacco virus, a tomato virus, a cucumber virus or a potato virus. In some embodiments, the plant virus is a mosaic virus, preferably Cowpea mosaic virus (CPMV). In some embodiments, the plant virus is a tobacco mosaic virus (TMV). In some embodiments, the plant virus is a tomato spotted wilt virus (TSWV). In some embodiments, the plant virus is a tomato yellow leaf curl virus (TYLCV). In some embodiments, the plant virus is a cucumber mosaic virus (CMV). In some embodiments, the plant virus is a potato virus Y (PVY).

In some embodiments, the protein is a bacteriophage protein, such as a protein from Salmonella virus P22, MS2, QBeta, PRR1, PP7, bacteriophage R17, bacteriophage fr, bacteriophage GA, bacteriophage SP, bacteriophage M11, bacteriophage MX1, bacteriophage NL95, bacteriophage f2 or Cb5. Additional relevant bacteriophage proteins are described in Lieknina et al., 2019.

In some embodiments, the protein is a protein from a virus which is common in mammals, in particular in humans. Without being bound by theory, such viruses may be better suited for expression in mammalian cells, in particular human cells, than viruses commonly found in non-mammalian organisms. Thus, in some embodiments, the virus is a hepatitis virus, such as a hepatitis B or E virus. In some embodiments, the virus is a norovirus. In some embodiments, the virus is a papilloma virus, such as Human Papilloma Virus (HPV), preferably HPV16 or HPV18. In some embodiments, the virus is a polyomavirus. In some embodiments, the virus is a parvovirus.

In some embodiments, the protein is a particle forming protein, such as ferritin, i301, replicase polyprotein 1a (pp1a) or a lumazine synthase. Other particle-forming proteins are known in the art and may also be used.

### First and Second Peptide Tags

Peptide pairs, consisting of a first peptide tag and a second peptide tag which are capable of binding to one another via the (spontaneous) formation of an isopeptide bond, are known in the art, or can be designed or obtained by methods known in the art, in particular as described in Zakeri et al., 2012, and in Zakeri et al., 2010.

The term “peptide tag” as used herein generally refers to a small peptide fragment which may be designed or derived directly from a protein which naturally forms an intramolecular isopeptide bond. Peptide tags may also be identified by using a known binding partner, for example derived from a protein naturally forming an intramolecular isopeptide bond, to screen a peptide library. The candidate peptide tags may thus be from a library, e.g. a peptide library, which can be screened for candidate peptide tags. They may also be designed in silico.

A peptide pair as understood herein thus consists of two peptide tags which can interact via the spontaneous formation of an isopeptide bond, or via an ester bond. Generally, these are also called a “tag and catcher” system, where the longer of the two peptide tags is termed “catcher” while the shorter of the two peptide tags is termed “tag”. For instance, the SpyTag/SpyCatcher system consists of a first peptide tag (SpyTag) and a second peptide tag (SpyCatcher).

In some embodiments, the peptide pair as understood herein consists of two peptide tags which can interact via the spontaneous formation of an ester bond. Useful peptide tags able to form such spontaneous ester bonds are further described in Young et al., 2017.

The “tag” may be between 5-50 amino acids in length e.g. from 10, 20, 30, 40 to 50 amino acids in length and may bind covalently via an isopeptide bond to a binding partner as defined herein. Thus, the “tag” may comprise one reactive residue involved in an isopeptide bond in the isopeptide protein used to design the binding partner (and the binding partner may comprise the other reactive residue involved in that bond), as described herein.

In some embodiments, the “tag” has a length between 7 and 47 amino acids, such as between 8 and 46 amino acids, such as between 9 and 45 amino acids, such as between 10 and 44 amino acids, such as between 11 and 43 amino acids, such as between 12 and 42 amino acids, such as between 13 and 41 amino acids, such as between 14 and 40 amino acids, such as between 15 and 39 amino acids, such as between 16 and 38 amino acids, such as between 17 and 37 amino acids, such as between 18 and 36 amino acids, such as between 19 and 35 amino acids, such as between 20 and 34 amino acids, such as between 21 and 33 amino acids, such as between 22 and 32 amino acids, such as between 23 and 31 amino acids, such as between 24 and 30 amino acids, such as between 25 and 29 amino acids, such as between 26 and 28 amino acids, such as 27 amino acids. In some embodiments, the “tag” has a length of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46 amino acids.

In some embodiments, the “catcher” is at least 20 amino acids in length. Preferably, the “catcher” has a length of 5 amino acids or more, such as 10 amino acids or more, such as 15 amino acids or more, such as 20 amino acids or more, such as 25 amino acids, such as 30 amino acids, such as 35 amino acids, such as 40 amino acids, such as 45 amino acids, such as 50 amino acids, such as 60, 70, 80, 90, 100, 125, 150, 175, 200, 225, 250, 275, 300, 325 or 350 amino acids or more. In preferred embodiments, the “catcher” is at least 20 amino acids in length. In some embodiments, the “catcher” is between 75 to 125 amino acids in length

Preferably, the “catcher” has an amino acid sequence which consists of more amino acid residues than the “tag”.

In some embodiments, one of the first and second peptide tags is selected from the group consisting of a SpyTag (SEQ ID NO: 1), a SdyTag (SEQ ID NO: 2), a SnoopTag (SEQ ID NO: 3), a PhoTag (SEQ ID NO: 4), an EntTag (SEQ ID NO: 5), a KTag, a BacTag (SEQ ID NO: 15), a Bac2Tag (SEQ ID NO: 16), a Bac3Tag (SEQ ID NO: 17), a Bac4Tag (SEQ ID NO: 18), a RumTrunkTag (SEQ ID NO: 13 or SEQ ID NO: 14), a Rum7Tag (SEQ ID NO: 12), a RumTag (SEQ ID NO: 6), a Rum2Tag (SEQ ID NO: 7), a Rum3Tag (SEQ ID NO: 8), a Rum4Tag (SEQ ID NO: 9), a Rum5Tag (SEQ ID NO: 10), a Rum6Tag (SEQ ID NO: 11) and a Bac5Tag (SEQ ID NO: 19). Nucleic acid sequences encoding said tags are as follows: SpyTag (SEQ ID NO: 35), SdyTag (SEQ ID NO: 36), SnoopTag (SEQ ID NO: 37), PhoTag (SEQ ID NO: 38), EntTag (SEQ ID NO: 39), KTag, BacTag (SEQ ID NO: 49), Bac2Tag (SEQ ID NO: 50), Bac3Tag (SEQ ID NO: 51), Bac4Tag (SEQ ID NO: 52), RumTrunkTag (SEQ ID NO: 47 or SEQ ID NO: 48), Rum7Tag (SEQ ID NO: 46), RumTag (SEQ ID NO: 40), Rum2Tag (SEQ ID NO: 41), Rum3Tag (SEQ ID NO: 42), Rum4Tag (SEQ ID NO: 43), Rum5Tag (SEQ ID NO: 44), Rum6Tag (SEQ ID NO: 45) and Bac5Tag (SEQ ID NO: 46).

In some embodiments, the other of the first and second peptide tags is selected from the group consisting of a SpyCatcher (SEQ ID NO: 55), a SdyCatcher (SEQ ID NO: 56), a SnoopCatcher (SEQ ID NO: 57) and an esther-forming split-protein pair. An example of an esther-forming split-protein pair is the fragment corresponding to amino acid residues 439-587 of cpe0147 (Uniprot B1R775) (SEQ ID NO: 34, DNA sequence: SEQ ID NO: 68) and the fragment corresponding to amino acid residues 565-587 of cpe0147 (Uniprot B1R775) (SEQ ID NO: 20; DNA sequence: SEQ ID NO: 54). Other first or second peptide tags are presented in SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33; the corresponding DNA sequences are SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, and SEQ ID NO: 67.

In some embodiments, the peptide pair comprises or consists of a SpyTag and a SpyCatcher. In some embodiments, the peptide pair comprises or consists of an SdyTag and an SdyCatcher. In some embodiments, the peptide pair comprises or consists of a SnoopTag and a SnoopCatcher.

In some embodiments, the peptide pair comprises or consists of truncated or modified versions of any of the above, i.e. further engineered peptide pairs, which however retain the ability to form an isopeptide bond.

A peptide tag may be altered, e.g. mutations or alterations may be introduced in any one or any two of the first or second peptide tag, i.e. in any one or any two of the tag and catcher. The peptide tag, i.e. the first, or the second peptide tag, should be able to covalently bind to a corresponding binding partner via an isopeptide bond, or via an ester bond, spontaneously. In this respect, each peptide tag preferably comprises one of the reactive amino acid residues involved in the formation of an isopeptide bond in the isopeptide protein. Hence, each peptide tag comprises only one reactive residue from the isopeptide bond and does not comprise both reactive residues involved. Further, if the peptide tag is modified or mutated, the reactive residue in that fragment preferably remains unchanged. This means that when a homologue of a peptide tag is used, the homologue preferably still contains the reactive residue which was originally present in the original peptide tag. In some embodiments, however, the reactive residue in that fragment is also changed if the peptide tag is modified or mutated.

Preferably, the reactive residue present in the tag is an asparagine or an aspartate residue, which can form an isopeptide bond with the reactive residue of the binding partner or modified binding partner (the catcher), as described above. Thus, one peptide tag contains one reactive residue while the other peptide tag contains the other reactive residue, and thus no single peptide tag contain both reactive residues.

In some embodiments, both reactive residues are involved in the formation of an isopeptide bond. In some embodiments, the reactive residue of the first peptide tag is different than the reactive residue of the second peptide tag. Preferably, the reactive residue present in the “catcher” is a lysine residue. In some embodiments, the reactive residue present in the “catcher” is an asparagine or an aspartate residue. Preferably, the reactive residue present in the “tag” is an asparagine or an aspartate residue. In some embodiments, the reactive residue present in the “tag” is a lysine residue. These residues together may form the isopeptide bond.

Without being bound by theory, a third residue may be involved in the formation of the isopeptide bond. While not directly participating in the bond, this third residue may mediate the formation of the bond. Typically, the third residue is a glutamate residue. The modified binding partner preferably comprises this third residue. In other words, the peptide tag, i.e. any of the first, second or third peptide tag, preferably does not comprise this third residue, which is instead present in the modified binding partner.

Consequently, as long as the peptide pair retains the ability to form an isopeptide bond, the peptide pair may comprise or consists of truncated or modified versions of any of the above mentioned peptide tags.

Thus, in some embodiments, one of the first and second peptide tags is selected from the group consisting of a SpyTag (SEQ ID NO: 1), a SdyTag (SEQ ID NO: 2), a SnoopTag (SEQ ID NO: 3), a PhoTag (SEQ ID NO: 4), an EntTag (SEQ ID NO: 5), a KTag, a BacTag (SEQ ID NO: 15), a Bac2Tag (SEQ ID NO: 16), a Bac3Tag (SEQ ID NO: 17), a Bac4Tag (SEQ ID NO: 18), a RumTrunkTag (SEQ ID NO: 13 or SEQ ID NO: 14), a Rum7Tag (SEQ ID NO: 12), a RumTag (SEQ ID NO: 6), a Rum2Tag (SEQ ID NO: 7), a Rum3Tag (SEQ ID NO: 8), a Rum4Tag (SEQ ID NO: 9), a Rum5Tag (SEQ ID NO: 10), a Rum6Tag (SEQ ID NO: 11), a Bac5Tag (SEQ ID NO: 19) and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of one of the first and second peptide tags may be selected from the group consisting of a SpyTag (SEQ ID NO: 35), SdyTag (SEQ ID NO: 36), SnoopTag (SEQ ID NO: 37), PhoTag (SEQ ID NO: 38), EntTag (SEQ ID NO: 39), KTag, BacTag (SEQ ID NO: 49), Bac2Tag (SEQ ID NO: 50), Bac3Tag (SEQ ID NO: 51), Bac4Tag (SEQ ID NO: 52), RumTrunkTag (SEQ ID NO: 47 or SEQ ID NO: 48), Rum7Tag (SEQ ID NO: 46), RumTag (SEQ ID NO: 40), Rum2Tag (SEQ ID NO: 41), Rum3Tag (SEQ ID NO: 42), Rum4Tag (SEQ ID NO: 43), Rum5Tag (SEQ ID NO: 44), Rum6Tag (SEQ ID NO: 45), Bac5Tag (SEQ ID NO: 46) and variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the other of the first and second peptide tags is selected from the group consisting of a SpyCatcher (SEQ ID NO: 21), a SdyCatcher (SEQ ID NO: 22), a SnoopCatcher (SEQ ID NO: 23), an esther-forming split-protein pair (such as the fragment corresponding to amino acid residues 439-587 of cpe0147 (Uniprot B1R775) (SEQ ID NO: 34, DNA sequence: SEQ ID NO: 68) and the fragment corresponding to amino acid residues 565-587 of cpe0147 (Uniprot B1R775) (SEQ ID NO: 20; DNA sequence: SEQ ID NO: 54)), SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33 and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the other of the first and second peptide tags may be selected from the group consisting of the DNA sequence of SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67 and variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of a SpyTag (SEQ ID NO: 1) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a SpyCatcher (SEQ ID NO: 21) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of a SpyTag (SEQ ID NO: 35) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and a SpyCatcher (SEQ ID NO: 55) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of an SdyTag (SEQ ID NO: 2) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and an SdyCatcher (SEQ ID NO: 22) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of an SdyTag (SEQ ID NO: 36) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and an SdyCatcher (SEQ ID NO: 56) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of a SnoopTag (SEQ ID NO: 3) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a SnoopCatcher (SEQ ID NO: 23) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of a SnoopTag (SEQ ID NO: 37) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and a SnoopCatcher (SEQ ID NO: 57) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

### Fusion of the First Peptide Tag to the Protein and Fusion of the Second Peptide Tag to the Antigen

Changing the position where the first peptide tag is fused to the protein may allow changing the orientation of the antigen on the particle. This may be performed to enable the best possible display of the most important epitopes of the antigen. The best possible orientation may be different from antigen to antigen.

Epitopes of specific monoclonal antibodies may be mapped on the antigen structure, whereby it is possible to determine which epitopes are accessible after conjugation of the antigen to the particle. Specifically, one may measure binding between a specific monoclonal antibody and the complex of the antigen bound to the particle (antigen:particle complex), such as by using ELISA or another affinity-measuring technique (e.g. Attana), and thereby determine the orientation of the antigen. Cryo-electron microscopy may also be used to determine the structure of the entire antigen:particle complex. If the antigen contains a functional binding epitope, binding-assays may be conducted to determine if the epitope is exposed or hidden in the final antigen:particle complex.

Changing the position where the second peptide tag is fused to the antigen will allow changing the orientation of the antigen on the particle. This may be performed to enable the best possible display of the most important epitopes of the antigen. The best possible orientation may be different from antigen to antigen.

In some embodiments, the first peptide tag is fused to the N-terminus of the protein. In other embodiments, the first peptide tag is fused to the C-terminus of the protein. In other embodiments, the first polynucleotide is inserted in-frame in the coding sequence of the protein. The fusion protein may comprise a linker between the first peptide tag and the protein.

Similarly, in some embodiments, the second peptide tag is fused to the N-terminus of the antigen. In other embodiments, the second peptide tag is fused to the C-terminus of the antigen. In other embodiments, the second polynucleotide is inserted in-frame in the coding sequence of the antigen. The fusion protein may comprise a linker between the second peptide tag and the antigen.

### Diseases and Medical Indications

The present invention is a novel, generic, and easy-to-use-approach to conjugate various antigens to a nanoparticle or VLP directly in the cell in which the nanoparticle or VLP is to be expressed, i.e. in vivo. Depending on the antigen, the present compositions can be used for prophylaxis and/or treatment of a wide range of diseases. The diseases which the present invention may be used for prophylaxis and/or treatment of include, but are not limited to, cancers, cardiovascular diseases, allergic diseases, chronic diseases, neurologic diseases, and/or infectious diseases. Antigens are typically peptides, polypeptides or proteins or fragments thereof, i.e. they comprise or consist of an amino acid sequence.

In some embodiments, an antigen which is associated with at least one cancer disease is linked to the protein, such as a particle-forming protein as described herein, via the interaction between the first peptide tag and second peptide tag. In a further embodiment the present compositions may be used for prophylaxis and/or treatment of the cancer and/or cancers which the antigen is associated with.

In some embodiments, an antigen which is associated with at least one cardiovascular disease is linked to the protein, such as a particle-forming protein as described herein, via the interaction between the first peptide tag and second peptide tag. In a further embodiment the present compositions can be used for prophylaxis and/or treatment of the cardiovascular disease and/or cardiovascular diseases which the antigen is associated with.

In some embodiments, an antigen which is associated with at least one allergic disease is linked to the protein, such as a particle-forming protein as described herein, via the interaction between the first peptide tag and second peptide tag. In a further embodiment the present compositions can be used for prophylaxis and/or treatment of the allergic disease and/or allergic diseases which the antigen is associated with.

In some embodiments, an antigen which is associated with at least one infectious disease is linked to the protein, such as a particle-forming protein as described herein, via the interaction between the first peptide tag and second peptide tag. In a further embodiment the present compositions can be used for prophylaxis and/or treatment of the infectious disease and/or infectious diseases which the antigen is associated with.

In some embodiments, an antigen which is associated with at least one chronic disease is linked to the protein, such as a particle-forming protein as described herein, via the interaction between the first peptide tag and second peptide tag. In a further embodiment the present compositions can be used for prophylaxis and/or treatment of the chronic disease and/or chronic diseases which the antigen is associated with.

In some embodiments, an antigen which is associated with at least one neurologic disease is linked to the protein, such as a particle-forming protein as described herein, via the interaction between the first peptide tag and second peptide tag. In a further embodiment the present compositions can be used for prophylaxis and/or treatment of the neurologic disease and/or neurologic diseases which the antigen is associated with.

In some embodiments, an antigen which is associated with at least one viral disease is linked to the protein, such as a particle-forming protein as described herein, via the interaction between the first peptide tag and second peptide tag. In a further embodiment the present compositions can be used for prophylaxis and/or treatment of the viral disease which the antigen is associated with. In some embodiments, the viral disease is caused by a coronavirus such as SARS-CoV-2, malaria, tuberculosis, HIV or influenza.

A non-exhaustive list of antigens which may be used with the present invention is outlined in Table 1 and Table 2. In addition, table 1 shows examples of specific diseases the antigens are associated with as well as examples of patient groups which may be in need of prophylaxis and/or treatment using the present compositions.

Relevant antigens include: hemagglutinin, GD2, EGF-R, CEA, CD52, CD21, neuraminidase, human melanoma protein gp100, human melanoma protein melan-A/MART1, HIV envelope protein, M2e, VAR2CSA, ICAM1, CSP, Dengue virus NS1, Dengue virus envelope protein, Chikungunya virus envelope protein, tyrosinase, HCV E2, NA17-A nt, MAGE-3, HPV 16 E7, HPV L2, PD1, PD-L1, CTLA-4, p53, hCG, Fel d1, EGRFvIII, endoglin, ANGPTL-3, CSPG4, CTLA-4, HER2, IgE, IL-1 beta, IL-5, IL-13, IL-17, IL-22, IL-31, IL-33, TSLP, NGF and (IHNV) G-protein, a lymphotoxin such as lymphotoxin α or β, a lymphotoxin receptor, a receptor activator of nuclear factor kB ligand, vascular endothelial growth factor VEGF, a VEGF receptor, IL-23 p19, ghrelin, CCL21, CXCL12, SDF-1, M-CSF, MCP-1, endoglin, GnRH, TRH, eotaxin, bradykinin, BLC, TNF-α, amyloid β peptide A, angiotensin, gastrin, progastrin, CETP, CCR5, C5a, CXCR4, Des-Arg-bradykinin, GnRH peptide, angiotensin peptide or TNF peptide.

In some embodiments, the antigen is an antigenic fragment or antigenic variant of hemagglutinin, GD2, EGF-R, CEA, CD52, CD21, neuraminidase, human melanoma protein gp100, human melanoma protein melan-A/MART1, HIV envelope protein, M2e, VAR2CSA, ICAM1, CSP, Dengue virus NS1, Dengue virus envelope protein, Chikungunya virus envelope protein, tyrosinase, HCV E2, NA17-A nt, MAGE-3, HPV 16 E7, HPV L2, PD1, PD-L1, CTLA-4, p53, hCG, Fel d1, EGRFvIII, endoglin, ANGPTL-3, CSPG4, CTLA-4, HER2, IgE, IL-1 beta, IL-5, IL-13, IL-17, IL-22, IL-31, IL-33, TSLP, NGF and (IHNV) G-protein, a lymphotoxin such as lymphotoxin α or β, a lymphotoxin receptor, a receptor activator of nuclear factor kB ligand, vascular endothelial growth factor VEGF, a VEGF receptor, IL-23 p19, ghrelin, CCL21, CXCL12, SDF-1, M-CSF, MCP-1, endoglin, GnRH, TRH, eotaxin, bradykinin, BLC, TNF-α, amyloid β peptide A, angiotensin, gastrin, progastrin, CETP, CCR5, C5a, CXCR4, Des-Arg-bradykinin, GnRH peptide, angiotensin peptide or TNF peptide. This antigenic fragment or antigenic variant may be better at inducing a stronger immune response than the corresponding antigen. Thus, in some embodiments, the protein sequence of the antigenic fragment or antigenic variant is a homologue of the corresponding antigen, having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, the antigenic fragment or antigenic variant is encoded by a polypeptide. Said polypeptide may consist or comprise of a nucleic acid sequence variant of the corresponding natural antigen, the nucleic acid sequence variant having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity to the corresponding antigen.

It is desirable that the cell upon transfection and expression of the antigen and the particle-forming protein, each fused to a tag as described herein, can form self-assembling particles displaying the antigen linked to the particle-forming protein. This may be assessed by relevant methods as known to the person skilled in the art, such as those disclosed in Example 2 and 3 of the present disclosure.

The compositions of the present invention may as well be used against other diseases and/or use other antigens than the ones listed herein.

In an embodiment of the present invention, the medical indication is selected from the group consisting of a cardiovascular disease, an immune-inflammatory disease, a chronic disease, a neurologic disease, an infectious disease and cancer. In a particular embodiment, the medical indication is an immune-inflammatory disease. In another particular embodiment, the medical indication is a cardiovascular disease. In another embodiment the medical indication is a chronic disease. In another embodiment the medical indication is a neurologic disease. In another embodiment, the medical indication is an infectious disease. In another embodiment, the medical indication is cancer.

In another embodiment, the antigen is a polypeptide, peptide and/or an antigenic fragment of a polypeptide associated with an abnormal physiological response, such as a cardiovascular disease and/or an allergic reaction/disease. In a particular embodiment the abnormal physiological response is a cancer.

In a further embodiment the antigen is a protein, peptide and/or an antigenic fragment associated with a medical indication as disclosed herein.

TABLE 1
Non-exhaustive list of antigens or parts hereof that could be used in treatment
of specific diseases/medical indications in various patient groups.

| Examples of antigens | Examples of a specific disease | Examples of patient group |
|---|---|---|
| (non-exhaustive) | (non-exhaustive) | (non-exhaustive) |
| Her2/Neu (ERBB2) | Breast cancer | Females overexpressing Her2 |
| Her2/Neu (ERBB2) | Gastric cancer | Males and females overexpressing Her2 |
| Her2/Neu (ERBB2) | Ovarian cancer | Females overexpressing Her2 |
| Her2/Neu (ERBB2) | Uterine serous | Postmenopausal Females |
| | carcinoma | overexpressing Her2 |
| Survivin | Cancer types | Males and non-pregnant females |
| | overexpressing Survivin | overexpressing Survivin |
| PCSK9 | Cardiovascular disease | Males and females with dyslipidemia |
| PCSK9 | Cardiovascular disease | Males and females with atherosclerosis |
| PCSK9 | Cardiovascular disease | Males and females with |
| | | hypercholesterolemia |
| ANGPTL3 | Cardiovascular disease | Males and females with dyslipidemia |
| ANGPTL3 | Cardiovascular disease | Males and females with atherosclerosis |
| ANGPTL3 | Cardiovascular disease | Males and females with |
| | | hypercholesterolemia |
| Interleukin-5 | Asthma | Males and females with eosinophilia |
| Interleukin-5 | nasal polyposis | Males and females with eosinophilia |
| Interleukin-5 | atopic dermatitis | Males and females with eosinophilia |
| Interleukin-5 | eosinophilic esophagitis | Males and females with eosinophilia |
| Interleukin-5 | Hypereosinophilic syndrome | Males and females with eosinophilia |
| Interleukin-5 | Churg-Strauss syndrome | Males and females with eosinophilia |
| Ag85A | Tuberculosis | Males and females with tuberculosis |
| PfRH5 | Malaria | Males and females with malaria |
| VAR2CSA | Malaria | Females with malaria |
| PfEMP1, CIDR1a | Malaria | Males and females with malaria |
| GLURP | Malaria | Males and females with malaria |
| MSP3 | Malaria | Males and females with malaria |
| Pfs25 | Malaria | Males and females with malaria |
| CSP | Malaria | Males and females with malaria |
| PfSEA-1 | Malaria | Males and females with malaria |
| Hemagglutinin HA | Influenza | Males and females with influenza |
| Interleukin-17 | Psoriasis | Males and females with Psoriasis |
| Interleukin-17 | Multiple sclerosis | Males and females with multiple |
| | | sclerosis |
| Interleukin-17 | Rheumatoid arthritis | Males and females with rheumatoid |
| | | arthritis |
| Interleukin-17 | Inflammatory bowel | Males and females with inflammatory |
| | diseases | bowel diseases |
| Interleukin-17 | Asthma | Males and females with Asthma |
| IL-33 | Asthma | Males and females with Asthma |
| IgE | Asthma | Males and females with Asthma |
| Gp160 | HIV | Males and females with HIV |
| Gp120 | HIV | Males and females with HIV |
| Gp40 | HIV | Males and females with HIV |
| GD2 | Cancer cell types expressing | Males and females with GD2 expressing |
| | GD2 (e.g. melanomas, | tumors. |
| | osteosarcoma and soft- | |
| | tissue sarcomas) | |
| EGF-R | Cancer cell types | Males and females with EGF-R |
| | expressing EGF-R (e.g. | expressing tumors. |
| | metastatic colorectal | |
| | and head and neck cancer) | |
| CEA | Cancer cell types expressing | Males and females with CEA expressing |
| | CEA (e.g. colon and rectal | tumors. |
| | cancer or pancreatic, breast, | |
| | ovary, or lung cancer. | |
| CD52 | Chronic lymphocytic leukemia | Males and females with chronic |
| | (CLL), cutaneous T-cell | lymphocytic leukemia (CLL), cutaneous |
| | lymphoma (CTCL) and | T-cell lymphoma (CTCL) and T-cell |
| | T-cell lymphoma or | lymphoma or multiple sclerosis. |
| | multiple sclerosis | |
| CD21 | B-cell cancers | Males and females with B-cell cancers |
| human | Cancer cell types expressing | Males and females with melanoma. |
| melanoma | human melanoma protein gp | |
| protein gp100 | 100 (e.g. Melanoma). | |
| human | Cancer cell types | Males and females with melanoma. |
| melanoma | expressing human | |
| protein melan- | melanoma protein | |
| A/MART-1 | melan-A/MART-1 (e.g. | |
| | Melanoma) | |
| tyrosinase | Melanoma | Males and females with melanoma |
| NA17-A nt | Melanoma | Males and females with melanoma |
| protein | | |
| MAGE-3 protein | melanoma, non-small cell | Males and females with melanoma, non- |
| | lung cancer, hematologic | small cell lung cancer or hematologic |
| | malignancies. | malignancies |
| p53 protein | Cancer cell types | Males and females with tumors |
| | expressing p53 | expressing p53 |
| HPV 16 E7 | Cancers of the cervix, | HPV infected males and females |
| protein | vulva, vagina, penis, | |
| | oropharynx and anus. | |
| HPV L2 | Cancers of the cervix, | HPV infected males and females |
| | vulva, vagina, penis, | |
| | oropharynx and anus. | |
| PD-L1 | Cancer types PD-L1 | Males and females with tumors |
| | | expressing PD-L1 |
| PD-L1 | Cancer types PD1 | Males and females with tumors |
| | | expressing PD1 |
| CTLA-4 | Cancer types CTLA-4 | Males and females with tumors |
| | | expressing CTLA-4 |
| hCG | Cancer cell types | Males and females with tumors |
| | expressing hCG | expressing hCG |
| Fel d1 | Cat allergy | Males and females allergic to cats |
| (IHNV) G-protein | Infectious | Salmon and trout infected with IHNV |
| | haematopoietic necrosis (IHN) | |

TABLE 2
Non-exhaustive list of diseases/medical indications and target
antigen/organisms of the present VLP/nanoparticle vaccine.

| Disease: | Target antigen/Organism: |
|---|---|
| Cancer: | Her2/Neu (ERBB2)/Homo Sapiens |
| | Survivin (Baculoviral IAP repeat-containing protein 5)/ |
| | Homo Sapiens |
| | GD2/Homo Sapiens |
| | EGF-R/Homo Sapiens |
| | CEA/Homo Sapiens |
| | CD52/Homo Sapiens |
| | human melanoma protein gp100/Homo Sapiens |
| | human melanoma protein melan-A/MART-1/Homo Sapiens |
| | tyrosinase/Homo Sapiens |
| | NA17-A nt protein/Homo Sapiens |
| | MAGE-3 protein/Homo Sapiens |
| | p53 protein/Homo Sapiens |
| | HPV 16 E7 protein/Human papillomavirus |
| | HPV L2 protein/Human papillomavirus |
| | PD1/Homo Sapiens |
| | PD-L1/Homo Sapiens |
| | CTLA-4/Homo Sapiens |
| | hCG/Homo Sapiens |
| | (IHNV) G-protein/Infectious haematopoietic necrosis virus |
| Cardiovascular disease: | PCSK9 (Proprotein convertase subtilisin/kexin type 9)/ |
| | Homo Sapiens |
| Asthma/Allergies: | IL-5 (Interleukin-5)/Homo Sapiens |
| | Fel d1/Felis catus |
| | IL-17/Homo sapiens |
| | IL-13/Homo sapiens |
| | IL-1B/Homo sapiens |
| | IgE/Homo sapiens |
| Tuberculosis: | Ag85A (Diacylglycerol cyltransferase/mycolyltransferase)/ |
| | Mycobacterium tuberculosis |
| Malaria: | Reticulocyte-binding protein homologue 5 (PfRH5)/ |
| | Plasmodium falciparum |
| | VAR2CSA (domain, ID1-ID2a)/Plasmodium falciparum |
| | CIDR1a domain of PfEMP1, Plasmodium falciparum |
| | Glutamate rich protein (GLURP)/Plasmodium falciparum |
| | Merozoite surface protein 3 (MSP3)/Plasmodium |
| | falciparum |
| | 25 kDa ookinete surface antigen (Pfs25)/Plasmodium |
| | falciparum |
| | Circumsporozoite protein (CSP)/Plasmodium falciparum |
| | Schizont egress antigen-1 (PfSEA-1)/Plasmodium |
| | falciparum |
| Multiple sclerosis | CD52/Homo sapiens |
| Contraception | hCG |
| Influenza | HA |

### Cancer and Associated Antigens

In 2012 more than 14 million adults were diagnosed with cancer and there were more than 8 million deaths from cancer, globally. Consequently, there is a need for efficient cancer therapeutics.

One characteristic of cancer cells is abnormal expression levels of genes and proteins. One example of a cancer associated gene is HER2, which is overexpressed in 20% of all breast cancers and is associated with increased metastatic potential and poor patient survival. Although cancer cells express cancer associated antigens in a way that immunologically distinguishes them from normal cells, most cancer associated antigens are only weakly immunogenic because most cancer associated antigens are “self” proteins which are generally tolerated by the host. The present compositions can be used to express, in a cell of a subject, particles displaying an antigen capable of activating the immune system to react against for example cancer associated antigens and overcome the immunological tolerance to such antigens. Different cancers are characterized by having different cancer associated antigens. Survivin is regarded to be overexpressed in most cancer cells and could also be used in the present invention. Therefore the present invention may be used in treatment/prophylaxis of most types of cancers that overexpress a tumor associated antigen.

Thereby the present invention provides compositions capable of activating the immune system to react against for example cancer associated antigens and overcome immunological tolerance to such antigens. In an embodiment the present compositions can be used for prophylaxis and/or treatment of the cancer which the antigen is associated with.

In another embodiment the present invention is used in treatment/prophylaxis of any type of cancer which overexpresses an antigen. The type of cancer which the invention may be used against is determined by the choice of antigen.

It is known that oncoviruses can cause cancer. Therefore in an embodiment the vaccine of the present invention comprises an oncovirus associated antigen linked to a protein, preferably a particle-forming protein, via the isopeptide bond or ester bond.

In a further embodiment the present compositions can be used for prophylaxis and/or treatment of the cancer which the antigen is associated with.

In an embodiment the antigen is a protein or peptide or an antigenic fragment of a polypeptide associated with a cancer selected from the group comprising of adrenal cancer, anal cancer, bile duct cancer, bladder cancer, bone cancer, brain/CNS tumors in adults, brain/CNS tumors in children, breast cancer, breast cancer in men, cancer in adolescents, cancer in children, cancer in young adults, cancer of unknown primary (CUP), Castleman disease, cervical cancer, colon/rectum cancer, endometrial cancer, esophagus cancer, Ewing family of tumors, eye cancer, gallbladder cancer, gastrointestinal carcinoid tumors, gastrointestinal stromal tumor, gestational trophoblastic disease, Hodgkin disease, Kaposi sarcoma, kidney cancer, laryngeal and hypopharyngeal cancer, leukemia, acute lymphocytic in adults, leukemia, acute myeloid leukemia, chronic lymphocytic leukemia, chronic myeloid leukemia, chronic myelomonocytic leukemia, leukemia in children, liver cancer, lung cancer, non-small cell lung cancer, small cell lung cancer, lung carcinoid tumor, lymphoma, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, non-Hodgkin lymphoma in children, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, adult soft tissue cancer sarcoma, skin cancer, basal and squamous cell skin cancer, melanoma skin cancer, Merkel cell skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor.

In some embodiments the cancer is selected from the group consisting of breast cancer, gastric cancer, ovarian cancer, and uterine serous carcinoma.

Linking the Her2/Neu (ERBB2) and/or Survivin or an antigenic fragment hereof to the VLP or nanoparticle as described herein forms a VLP or nanoparticle which is capable of activating the immune system to react against for example cells with high Her2/Neu (ERBB2) and/or Survivin expression and overcome immunological tolerance. In an embodiment the Her2/Neu (ERBB2) and/or Survivin can be used for prophylaxis and/or treatment of the herein disclosed cancer disease and/or other cancer diseases. Using a similar reasoning other cancer disease associated antigens may be used against any cancer disease. Such antigens may be chosen from the group consisting of interleukin-17, hemagglutinin, GD2, EGF-R, CEA, CD52, CD21, human melanoma protein gp100, human melanoma protein melan-A/MART1, tyrosinase, NA17-A nt, MAGE-3, HPV 16 E7, HPV L2, PD1, PD-L1, CTLA-4, p53, hCG, Fel d1 and (IHNV) G-protein.

In an embodiment the antigen of the present invention is Her2/Neu (ERBB2) and/or Survivin or an antigenic fragment hereof, wherein the antigen is associated with and directed against at least one of the herein disclosed types of cancers. In an embodiment the antigen of the present disclosure is interleukin-17, hemagglutinin, GD2, EGF-R, CEA, CD52, CD21, human melanoma protein gp100, human melanoma protein melan-A/MART1, tyrosinase, NA17-A nt, MAGE-3, HPV 16 E7, HPV L2, PD1, PD-L1, CTLA-4, HPV L2, PD1, PD-L1, CTLA-4, p53, hCG, Fel d1 and (IHNV) G-protein or an antigenic fragment thereof, wherein the antigen is associated with and directed against at least one of the herein disclosed types of cancers.

### Cardiovascular Diseases and Associated Antigens

An estimated 17.3 million people died from cardiovascular diseases in 2008, representing 30% of all global deaths. Addressing risk factors such as tobacco use, unhealthy diet and obesity, physical inactivity, high blood pressure, diabetes and raised lipids are important for prevention of cardiovascular diseases. However, the need for preventive pharmaceutical measures is increasingly important. The present invention may be used in treatment/prophylaxis of most types of cardiovascular diseases. The type of cardiovascular disease which the invention may be used against is decided by the choice of antigen.

In an embodiment of the invention the antigen is a protein or peptide or an antigenic fragment of a polypeptide associated with a disease selected from the group comprising a lipid disorder such as hyperlipidemia, type I, type II, type III, type IV, or type V hyperlipidemia, secondary hypertriglyceridemia, hypercholesterolemia, familial hypercholesterolemia, xanthomatosis, cholesterol acetyltransferase deficiency, an arteriosclerotic condition (e.g., atherosclerosis), a coronary artery disease, a cardiovascular disease.

In an embodiment of the invention the antigen is a protein or peptide or an antigenic fragment of a polypeptide associated with a cardiovascular disease. In a further embodiment the cardiovascular disease is selected from the group consisting of dyslipidemia, atherosclerosis, and hypercholesterolemia.

One example of a polypeptide associated with a cardiovascular disease is PCSK9 which acts in cholesterol homeostasis. Blockage of PCSK9 has medical significance and can lower the plasma and/or serum low-density lipoprotein cholesterol (LDL-C) levels. Reducing LDL-C reduces the risk of for example heart attacks.

Linking the PCSK9 antigen to the VLP or nanoparticle forms a PCSK9-VLP/nanoparticle based vaccine which is capable of activating the immune system to produce antibodies that bind PCSK9 and either clear PCSK9 from the bloodstream or hinders binding of PCSK9 to the LDL receptor, thereby lowering the LDL-C levels and the risk of heart attacks. In an embodiment, the present compositions can be used for prophylaxis and/or treatment of the herein disclosed cardiovascular disease and/or other cardiovascular diseases. Using a similar reasoning other cardiovascular disease associated antigens may be used against any cardiovascular disease.

In a preferred embodiment the antigen comprises PCSK9 or an antigenic fragment hereof, wherein the antigen is associated with and directed against at least one of the herein disclosed cardiovascular disease and/or other cardiovascular diseases.

Another example of a polypeptide associated with a cardiovascular disease is ANGPTL3 which acts in cholesterol homeostasis. Blockage of ANGPTL3 has medical significance and can lower the plasma and/or serum low-density lipoprotein cholesterol (LDL-C) levels. Reducing LDL-C reduces the risk of for example heart attacks.

Linking the ANGPTL3 antigen to the VLP or nanoparticle forms a ANGPTL3-VLP/nanoparticle based vaccine which is capable of activating the immune system to produce antibodies that bind ANGPTL3 and either clear ANGPTL3 from the bloodstream or hinders binding of ANGPTL3 to the LDL receptor, thereby lowering the LDL-C levels and the risk of heart attacks. In an embodiment, the present compositions can be used for prophylaxis and/or treatment of the herein disclosed cardiovascular disease and/or other cardiovascular diseases. Using a similar reasoning other cardiovascular disease associated antigens may be used against any cardiovascular disease.

In a preferred embodiment the antigen comprises ANGPTL3 or an antigenic fragment hereof, wherein the antigen is associated with and directed against at least one of the herein disclosed cardiovascular disease and/or other cardiovascular diseases.

### Immune-Inflammatory Diseases and Associated Antigens

The prevalence of immune-inflammatory diseases worldwide is rising dramatically in both developed and developing countries. According to World Health Organization statistics, hundreds of millions of subjects in the world suffer from allergic rhinitis and it is estimated that 300 million have asthma markedly affecting the quality of life of these individuals and negatively impacting the socio-economic welfare of society. Interleukin 5 (IL-5) has been shown to play an instrumental role in eosinophilic inflammation in various types of allergies, including severe eosinophilic asthma. Eosinophils are regulated in terms of their recruitment, activation, growth, differentiation and survival by IL-5 which, consequently, has identified this cytokine as a primary target for therapeutic interventions.

Linking an IL-5 antigen or a fragment hereof to the particle-forming protein of the present invention forms an IL-5-VLP/nanoparticle based vaccine which is capable of activating the immune system to react against IL-5. Consequently an IL-5-based composition described in the present invention may be used in the treatment/prophylaxis of eosinophilic asthma or other immune-inflammatory diseases. Other immune-inflammatory disease associated antigens (e.g. IgE or interleukin 17 or IL-17) may be used by the present invention using a similar reasoning. Consequently an IL-17-based vaccine described in the present invention may be used in the treatment/prophylaxis of eosinophilic asthma or other immune-inflammatory diseases. The type of asthma or allergy or other immune-inflammatory disease which the invention may be used against is decided by the choice of antigen. In an embodiment the antigen is a protein or peptide or an antigenic fragment of a polypeptide associated with one or more asthma or immune-inflammatory diseases disclosed herein. In a preferred embodiment the asthma or immune-inflammatory disease is selected from the group consisting of eosinophilic asthma, allergy, nasal polyposis, atopic dermatitis, eosinophilic esophagitis, hypereosinophilic syndrome, and Churg-Strauss syndrome.

In a preferred embodiment the antigen comprises IL-5, IL-17 or an antigenic fragment hereof, wherein the antigen is associated with and directed against at least one of the herein disclosed asthma or allergy diseases and/or other immune-inflammatory diseases.

### Infectious Diseases and Associated Antigens

Tuberculosis and malaria are two major infectious diseases. In 2012, an estimated 207 million cases of malaria occurred which resulted in more than 500.000 deaths. Also in 2012, an estimated 8.6 million people developed tuberculosis and 1.3 million died from the disease. The current methods of treatment are insufficient and some have resulted in drug resistance. Consequently there is a need for new and efficient drugs for treatment/prophylaxis of tuberculosis and malaria. Linking a malaria or tuberculosis associated-antigen or a fragment hereof to the VLP or nanoparticle of the present invention forms a VLP or nanoparticle based vaccine which is capable of activating the immune system to react against for example malaria or tuberculosis. Using a similar line of reasoning the present invention may be used in treatment/prophylaxis of most infectious disease. The type of infectious disease which the invention may be used against is decided by the choice of antigen.

The year 2020 has been marked by the SARS-CoV-2/COVID-19 pandemic, resulting in millions of infected subjects worldwide, and this infectious disease is thus of great interest. Influenza is another infectious disease of interest.

In an embodiment the antigen fused to the second peptide tag of the present invention is a protein or peptide or an antigenic fragment of a polypeptide associated with an infectious disease such as tuberculosis and/or malaria.

In one embodiment an antigen from Plasmodium falciparum is fused to the second peptide tag for use in treatment/prophylaxis of malaria.

In a further embodiment an antigen from Mycobacterium tuberculosis is fused to the second peptide tag for use in treatment/prophylaxis of tuberculosis.

In a further embodiment the antigen is selected from the group consisting of Ag85A from Mycobacterium tuberculosis, PfRH5 from Plasmodium falciparum, VAR2CSA (domain, ID1-ID2a) from Plasmodium falciparum, CIDR1a domain, of PfEMP1 from Plasmodium falciparum, GLU RP from Plasmodium falciparum, MSP3 from Plasmodium falciparum, Pfs25 from Plasmodium falciparum, CSP from Plasmodium falciparum, and PfSEA-1 from Plasmodium falciparum or an antigenic fragment of the disclosed antigens. In another embodiment the antigen comprises a fusion construct between MSP3 and GLURP (GMZ2) from Plasmodium falciparum.

In a further embodiment the antigen is a hemagglutinin (HA) antigen from the influenza virus or an antigenic fragment thereof.

In another embodiment the antigen of the present invention comprises a protein, or an antigenic fragment hereof, from the pathogenic organism which causes the infectious disease.

In one embodiment, the antigen is a protein, peptide and/or an antigenic fragment of Severe Acute Respiratory Syndrome Coronavirus 2 (SARS-CoV-2). Thus, in some embodiments, the antigen is a protein, peptide and/or an antigenic fragment of a SARS-CoV-2 envelope protein. In some embodiments, the antigen is a protein, peptide and/or an antigenic fragment of a SARS-CoV-2 spike protein. In some embodiments, the antigen is a protein, peptide and/or an antigenic fragment of a SARS-CoV-2 nucleocapsid protein. In some embodiments, the antigen is a protein, peptide and/or an antigenic fragment of a SARS-CoV-2 envelope protein. In specific embodiments, the antigen is amino acids 319-591 of the SARS-CoV-2 spike protein RBD (GenBank accession number: QIA20044.1). Said antigen may be fused to a catcher or a tag, and may further comprise a C-tag purification tag.

Thus, in some embodiments, the composition as described herein comprises:
i. a first polynucleotide encoding a protein fused to a first peptide tag; and
ii. a second polynucleotide encoding a SARS-CoV-2 antigen fused to a second peptide tag,
   wherein the antigen and the protein upon expression in a cell are linked via an isopeptide bond, or an ester bond, between the first peptide tag and the second peptide tag, whereby i-ii form a particle displaying said SARS-CoV-2 antigen.

In some embodiments, the composition as described herein comprises:
i. a first polynucleotide encoding a protein fused to a SpyCatcher; and
ii. a second polynucleotide encoding a SARS-CoV-2 antigen fused to a SpyTag.

In some embodiments, the composition as described herein comprises:
i. a first polynucleotide encoding a protein fused to an SdyCatcher; and
ii. a second polynucleotide encoding a SARS-CoV-2 antigen fused to an SdyTag.

In some embodiments, the composition as described herein comprises:
i. a first polynucleotide encoding a protein fused to a SnoopCatcher; and
ii. a second polynucleotide encoding a SARS-CoV-2 antigen fused to a SnoopTag.

In some embodiments, the composition as described herein comprises:
i. a first polynucleotide encoding a protein fused to a SpyTag; and
ii. a second polynucleotide encoding a SARS-CoV-2 antigen fused to a SpyCatcher.

In some embodiments, the composition as described herein comprises:
i. a first polynucleotide encoding a protein fused to an SdyTag; and
ii. a second polynucleotide encoding a SARS-CoV-2 antigen fused to an SdyCatcher.

In some embodiments, the composition as described herein comprises:
i. a first polynucleotide encoding a protein fused to a SnoopTag; and
ii. a second polynucleotide encoding a SARS-CoV-2 antigen fused to a SnoopCatcher.

In one embodiment the antigen is a protein, peptide and/or an antigenic fragment of an influenza virus.

### Antigens Known to be Difficult to Express in Heterologous Expression Systems

Some antigens are difficult to express in a heterologous expression system (for example mammalian antigens produced in E. coli). A number of antigens are also present as multi-protein complexes, further complicating production and formulation. Protein degradation or aggregation during production and coupling can also cause issues for particle display, in particular for VLP display. This can result in antigens being insoluble and thus very complex or impossible to produce in vitro and used as vaccine for the VLP technology.

These antigens could thus benefit from being produced by DNA/mRNA in vivo and directly coupled to VLP in vivo. This would avoid the need to first produce and purify the protein/protein complex, and also avoids lengthy storage in potentially non-optimal buffers. Here are some examples of few antigens that could benefit from such a technology:

Interleukins: In the context of allergy and asthma, levels of interleukins are involved in the severity of the disease. The particle technology, in particular the VLP technology, allows for a breach of immune tolerance and thus to control levels of interleukins, thus alleviating some of the disease's symptoms. However, a number of interleukins are difficult to produce (particularly in E. coli, but also in a range of other expression systems) and often are insoluble. Among these, IL-13, IL-31 and IL-17A could benefit from the present mRNA/DNA technology.

Thus, in some embodiments, the antigen is IL-13. In some embodiments, the antigen is IL-31. In some embodiments, the antigen is IL-17A.

Similarly, PCSK9 is involved in cholesterol levels, thus research has been focusing on making a PCSK9 vaccine. In the SARS-CoV-2 pandemic, the mutation rate of the virus is quite high, thus, new vaccines might be needed in order to protect against the different variants. However, both of these antigens need to be produced in eukaryotic cells, making the production line costly and time consuming. Furthermore, PCSK9 has proven extremely difficult to couple stably to particles, including VLPs, with protein aggregation and degradation issues causing severe delays in development and requiring novel PCSK9 designs for success. This antigen could thus benefit from mRNA/DNA delivery technology, in order to cut down on the production time and cost.

Thus, in some embodiments, the antigen is PCSK9.

Finally, HIV trimers and Flu stem trimers are also difficult proteins to couple to particles, such as VLPs, as they are quite large proteins, and have intrinsic stability issues for the artificially designed stem trimers. It is also known from literature that a number of HIV variant sequences has proven impossible to incorporate in stem-only HIV vaccine designs due to instability issues (Zhang et al., 2021). In an in vivo system, where these antigens would be delivered as DNA/mRNA (where the coupling efficiency and pressure would be different while also removing the need to work with the unstable antigens for long periods before coupling to the particle), DNA/mRNA particle technology may be the solution.

Thus, in some embodiments, the antigen is an HIV trimer. In some embodiments, the antigen is an influenza virus trimer.

### Expression Systems

Herein is also provided an expression system comprising:
i. a first polynucleotide encoding a protein fused to a first peptide tag; and
ii. a second polynucleotide encoding an antigen fused to a second peptide tag,
   wherein upon expression of the first and second polynucleotides in a cell, the antigen and the protein are linked via an isopeptide bond, or an ester bond, between the first peptide tag and the second peptide tag, whereby i-ii form a particle displaying said antigen.

The expression system may consist of or comprise a polycistronic RNA construct and/or a DNA construct, from which the transcribed mRNA is polycistronic. Thus, in some embodiments, the first and the second polynucleotides of the expression system are encoded on the same ribonucleic acid molecule. In some embodiments, the first and the second polynucleotides of the expression system lie within the same open reading frame, whereby only one promoter sequence is needed to transcribe both polynucleotides. In some embodiments, the first and the second polynucleotides of the expression system lie within separate open reading frames and may thus be regulated by separate promoters.

The first peptide tag, the second peptide tag, the protein and/or the antigen may be as defined herein elsewhere.

The term “expression system” refers to a genetic construct designed to produce a protein and/or an RNA inside a cell. Thus, the expression system may comprise RNA and/or DNA, which is translated or transcribed to a protein or DNA, respectively, inside the cell.

The expression system may comprise the sequences necessary for gene expression in the cell. These may include a promoter, a translation initiation sequence such as a ribosomal binding site, a start codon, a termination codon, and a transcription termination sequence. There are differences in the enzymes responsible for protein synthesis between prokaryotes and eukaryotes, therefore the expression vectors must comprise elements for expression that are appropriate for the chosen host. For example, prokaryotic expression systems may comprise a Shine-Dalgarno sequence at the translation initiation site for the binding of ribosomes, while eukaryotic expression systems may contain a Kozak consensus sequence.

The expression system may additionally comprise a marker, such as a selectable marker, i.e. a gene that confers a trait suitable for artificial selection, whereby cells comprising the expression system may be selected for, or a screenable marker, such as a reporter gene, i.e. a gene that allows for differentiation between cells comprising or not comprising the expression system, whereby cells comprising the expression system may be identified. Examples of such markers include antibiotic resistance genes, auxotrophic markers and genes expressing detectable compounds, such as coloured and/or fluorescent compounds.

In some embodiments, the first polynucleotide and the second polynucleotide are both DNA polynucleotides. In some embodiments, the first polynucleotide and the second polynucleotide are both RNA polynucleotides. In some embodiments, the first polynucleotide or the second polynucleotide is a DNA polynucleotide and the other is an RNA polynucleotide.

The first polynucleotide and/or the second polynucleotide may be under the control of a promoter, such as an inducible promoter or a constitutive promoter. The first and/or the second polynucleotide may each be under the control of a first and/or second promoter, respectively, which may be identical or different. They may also be under the control of a single promoter.

The first and the second polynucleotides of the expression system may be comprised within the same molecule. The first and the second polynucleotides of the expression system may alternatively be comprised within different molecules, such as within two or more separate molecules.

The first and/or the second polynucleotide may further comprise a secretion or excretion signal to obtain a fusion protein comprising such a signal, whereby the protein fused to the first peptide tag and/or the antigen fused to the second peptide tag is secreted or excreted from the endoplasmic reticulum and optionally also from the cell.

The present expressions systems can be used for prophylaxis and/or treatment of a wide range of diseases as disclosed herein above.

### Cells and Host Cells

The invention further relates to a cell, such as a host cell, comprising a polynucleotide and/or an expression system as disclosed herein. The polynucleotide and/or expression system may have a sequence that is codon-optimised. Codon optimisation methods are known in the art and allow optimised expression in a heterologous host organism or cell. In an embodiment the cell may be selected from the group comprising bacteria, yeast, fungi, plant, mammalian and/or insect cells.

Methods for expressing a first polypeptide and/or a second polypeptide in a cell, such as a host cell, are known in the art. The first or second polypeptide may be heterologously expressed from corresponding polynucleotide sequences cloned into the genome of the cell or they may be comprised within a vector. For example, a first and/or second polynucleotide coding for the first and/or second polypeptide is cloned into the genome, and a first and/or second polynucleotide coding for the first and/or second polypeptide is comprised within a vector transformed or transfected into the cell.

Expression of the first and second polypeptides in the cell may occur in a transient manner. When the polynucleotide encoding one of the polypeptides is cloned into the genome, an inducible promoter may be cloned as well to control expression of the polypeptides. Such inducible promoters are known in the art. Alternatively, genes coding for suppressors of gene silencing may also be cloned into the genome or into a vector transfected within the cell.

Also provided herein is thus a cell expressing:
i. a first polynucleotide encoding a protein fused to a first peptide tag, preferably as defined in any one of the preceding claims; and
ii. a second polynucleotide encoding an antigen fused to a second peptide tag, preferably as defined in any one of the preceding claims;
   wherein the antigen and the protein upon expression in a cell are linked via an isopeptide bond, or an ester bond, between the first peptide tag and the second peptide tag, whereby i-ii form a particle displaying said antigen.

In some embodiments, the cell is a bacterial cell. In some embodiments, the cell is a yeast cell. In some embodiments, the cell is a fungal cell. In some embodiments, the cell is a plant cell. In some embodiments, the cell is a mammalian cell, such as a human cell. In some embodiments, the cell is an insect cell.

In a particular embodiment the cell, such as the host cell, may be selected from the group comprising Escherichia coli, Spodoptera frugiperda (sf9), Trichoplusia ni (BTI-TN-5B1-4), Pichia Pastoris, Saccharomyces cerevisiae, Hansenula polymorpha, Drosophila Schneider 2 (S2), Lactococcus lactis, Chinese hamster ovary (CHO), Human Embryonic Kidney 293, Nicotiana tabacum cv. Samsun NN and Solanum tuberosum cv. Solara. Thus in an embodiment, the cell is Escherichia coli. In another embodiment, the cell is Spodoptera frugiperda. In another embodiment, the cell is Pichia Pastoris. In another embodiment, the cell is Saccharomyces cerevisiae. In another embodiment, the cell is Hansenula polymorpha. In another embodiment, the cell is Drosophila Schneider 2. In another embodiment, the cell is Lactococcus lactis. In another embodiment, the cell is Chinese hamster ovary (CHO). In another embodiment, the cell is Human Embryonic Kidney 293. In another embodiment, the cell is Trichoplusia ni (BTI-TN-5B1-4). In another embodiment, the cell is Nicotiana tabacum cv. Samsun NN. In another embodiment, the cell is Solanum tuberosum cv. Solara.

In some embodiments, the cell, such as the host cell, expresses:
i. a first polynucleotide encoding a protein fused to a first peptide tag; and
ii. a second polynucleotide encoding an antigen fused to a second peptide tag,
   wherein the antigen and the protein upon expression in a cell are linked via an isopeptide bond, or an ester bond, between the first peptide tag and the second peptide tag, whereby i-ii form a particle displaying said antigen, and wherein the cell is selected from the group comprising bacteria, yeast, fungi, plant, mammalian and/or insect cells.

The present cells, such as host cells, can be used for prophylaxis and/or treatment of a wide range of diseases as disclosed herein above.

### EXAMPLES

### Example 1—Materials and Methods for Examples 2 and 3

Gene sequences of Spytagged Hepatitis B core antigen, Spytagged Acinobacter bacteriophage AP205 major coat protein and SpyCatcher-fused enhanced green fluorescent protein (eGFP) were codon optimized and synthesised by Geneart.

### Sequences:

>pVax1_SpyTag-AP205
(SEQ ID NO: 85, DNA sequence in SEQ ID NO: 88)
AHIVMVDAYKPTKGSGTAGGGSGSANKPMQPITSTANKIVWSDPTRLSTT
FSASLLRQRVKVGIAELNNVSGQYVSVYKRPAPKPEGCADACVIMPNENQ
SIRTVISGSAENLATLKAEWETHKRNVDTLFASGNAGLGFLDPTAAIVSS
DTTA*
>pVAX1_HBc-SpyTag
(SEQ ID NO: 86, DNA sequence in SEQ ID NO: 89)
MDIDPYKEFGASVELLSFLPSDFFPSIRDLLDTASALYREALESPEHVSP
HHTALRQAILCWGELMNLATWVGSNLEDPASRELVVSYVNVNMGLKLRQI
LWFHISCLTFGRETVLEYLVSFGVWIRTPTAYRPPNAPILSTLPETTVVG
GGGGSPGGGTPSPGGGGSQSPGGGGSQSGESQCGSAHIVMVDAYKPTK*
>pVAX1-SPYCEGFP
(SEQ ID NO: 87, DNA sequence in SEQ ID NO: 90)
GAMVDTLSGLSSEQGQSGDMTIEEDSATHIKFSKRDEDGKELAGATMELR
DSSGKTISTWISDGQVKDFYLYPGKYTFVETAAPDGYEVATAITFTVNEQ
GQVTVNGKATKGDAHIGGSGSMVSKGEELFTGVVPILVELDGDVNGHKFS
VSGEGEGDATYGKLTLKFICTTGKLPVPWPTLVTTLTYGVQCFSRYPDHM
KQHDFFKSAMPEGYVQERTIFFKDDGNYKTRAEVKFEGDTLVNRIELKGI
DFKEDGNILGHKLEYNYNSHNVYIMADKQKNGIKVNFKIRHNIEDGSVQL
ADHYQQNTPIGDGPVLLPDNHYLSTQSALSKDPNEKRDHMVLLEFVTAAG
ITLGMDELYK*

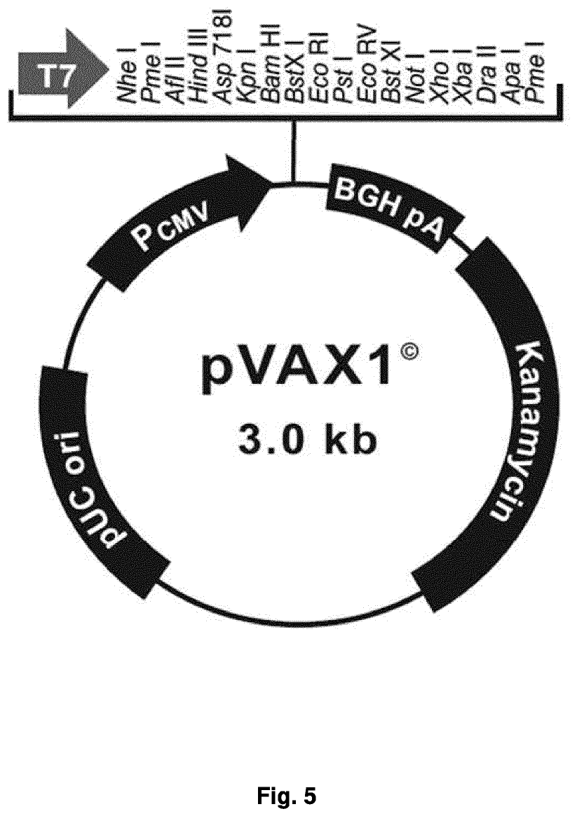

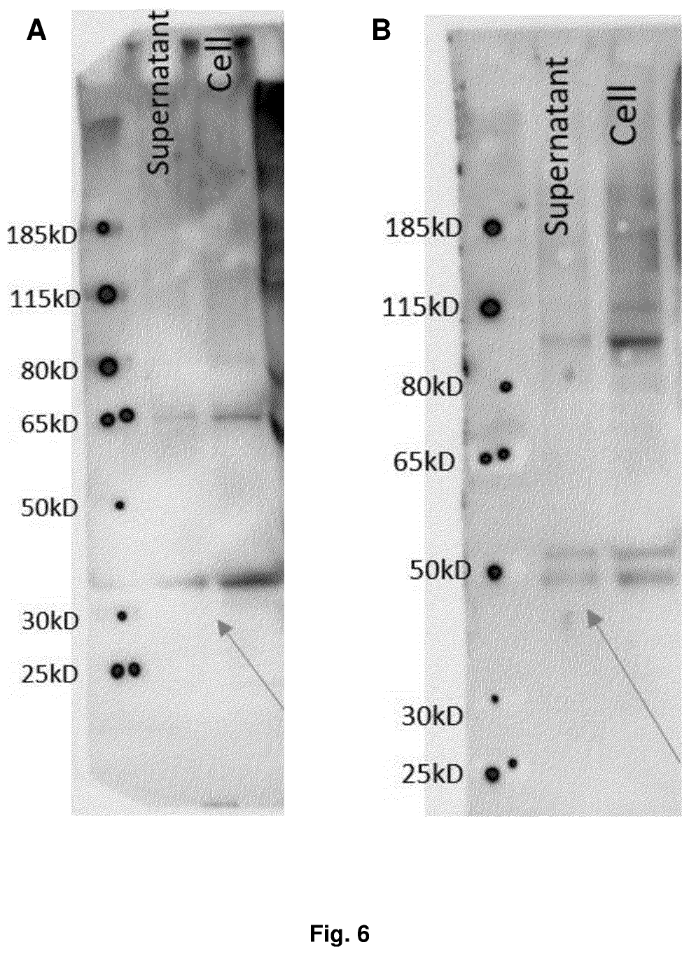

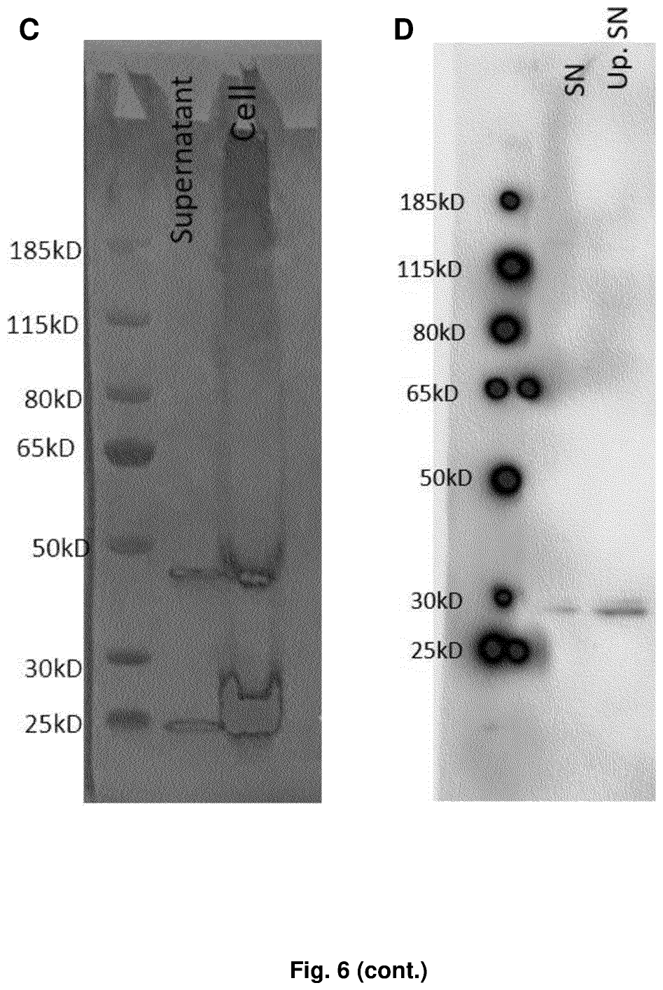

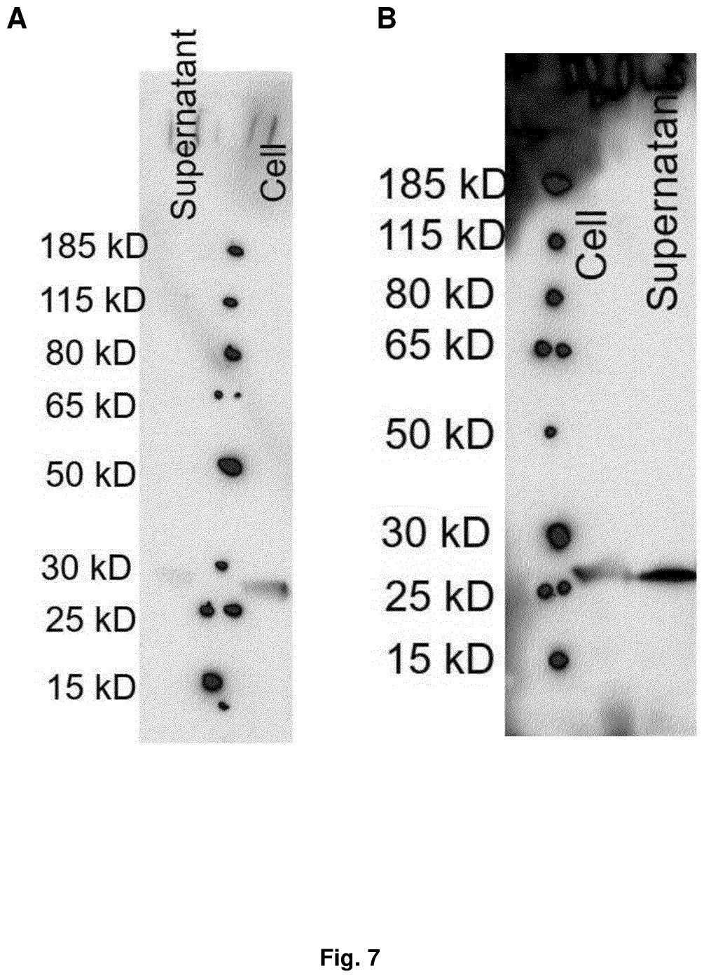

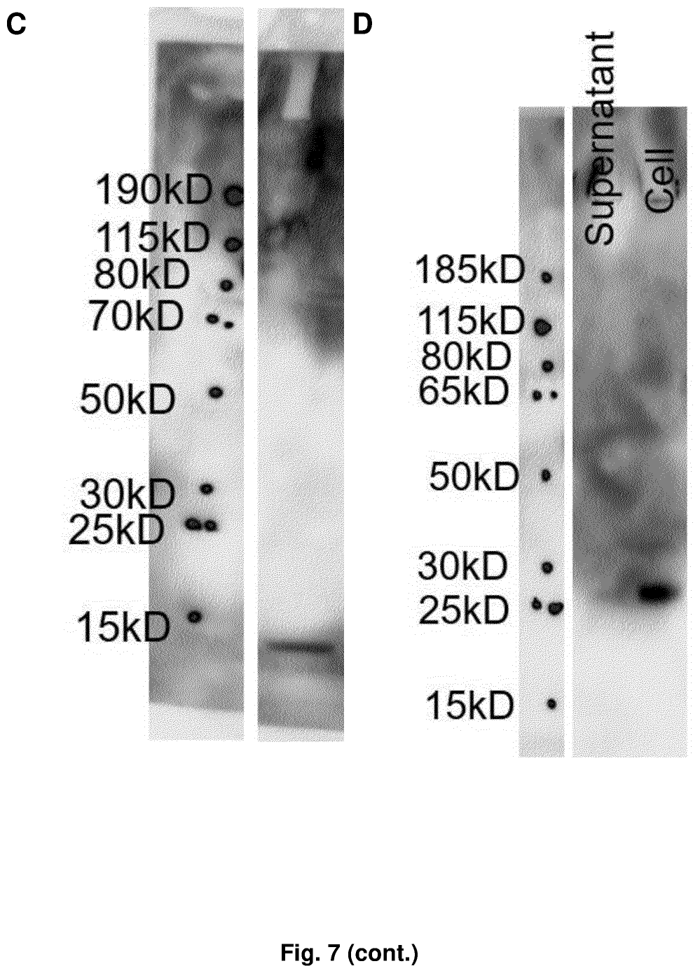

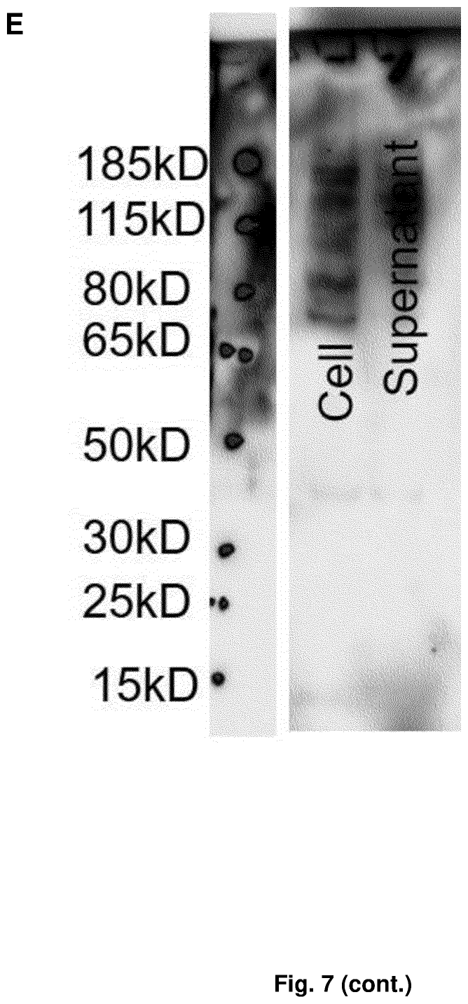

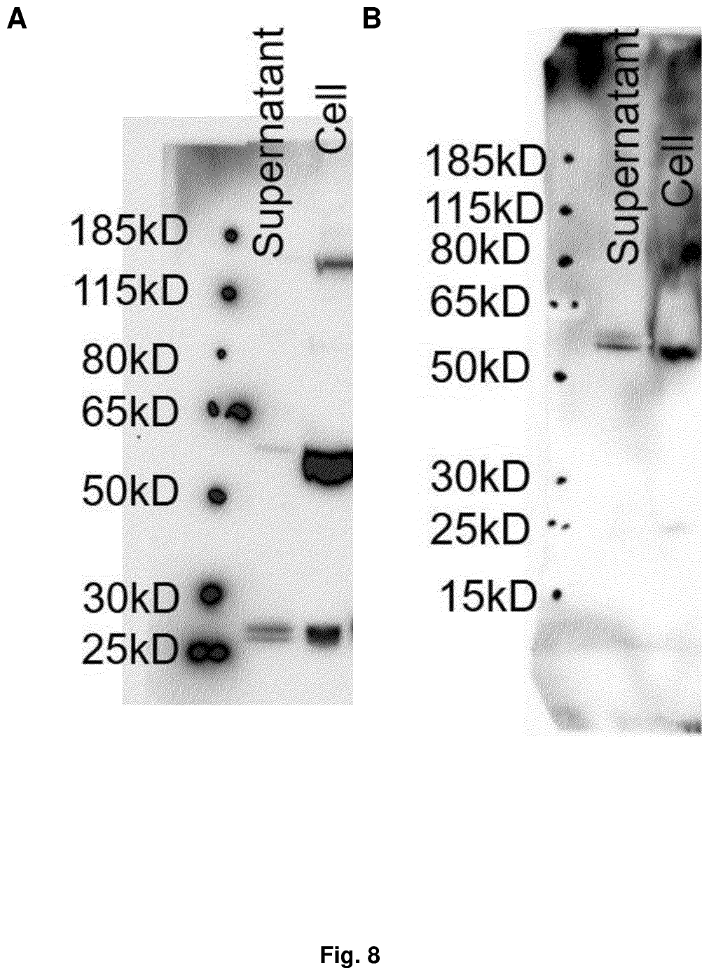

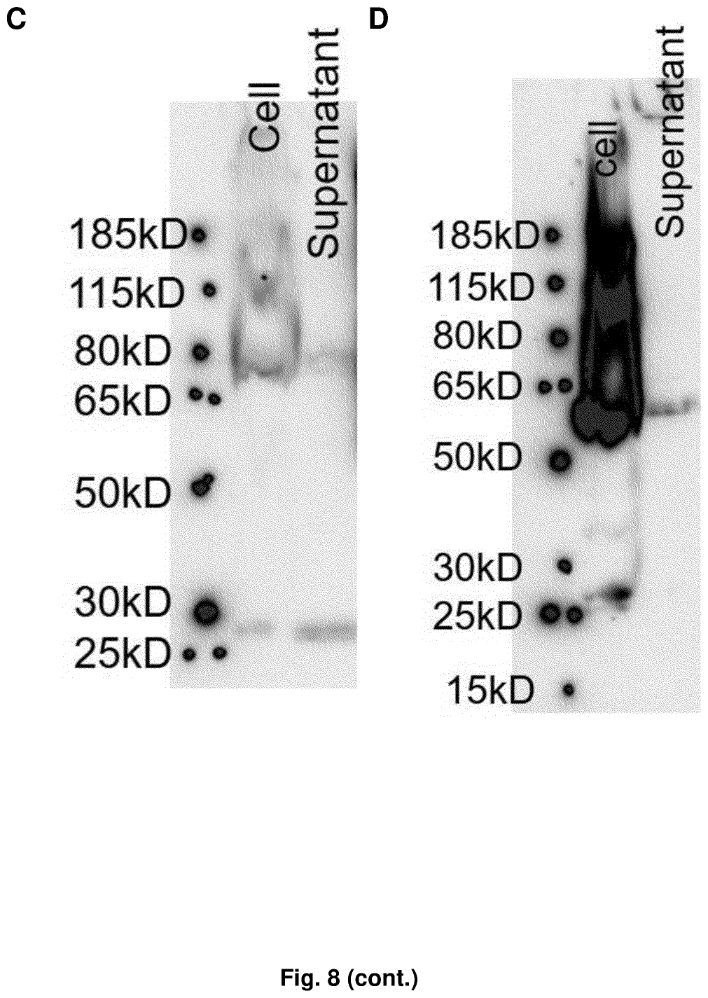

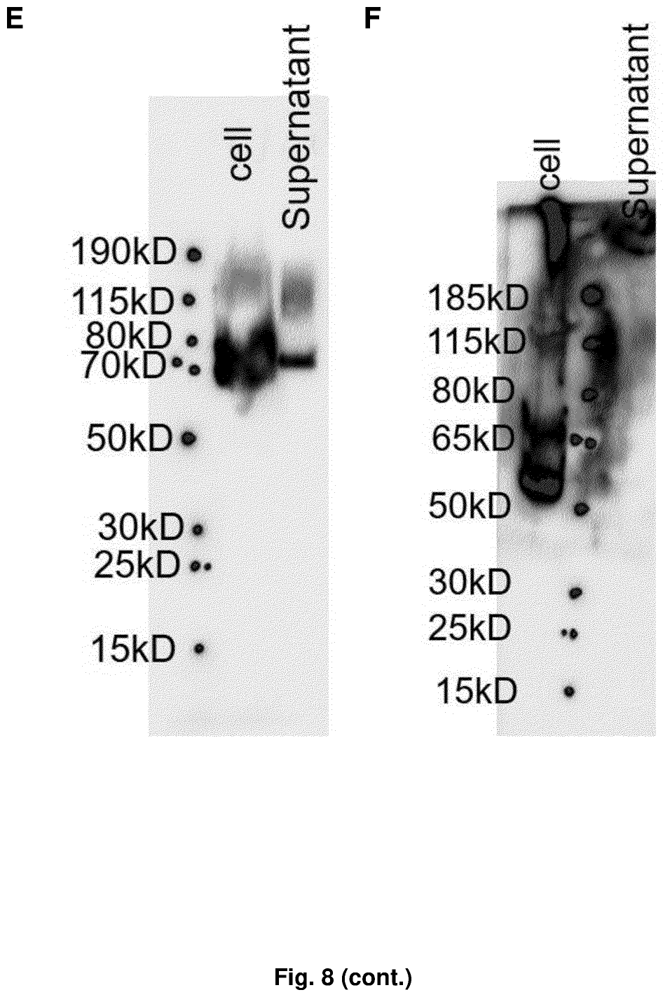

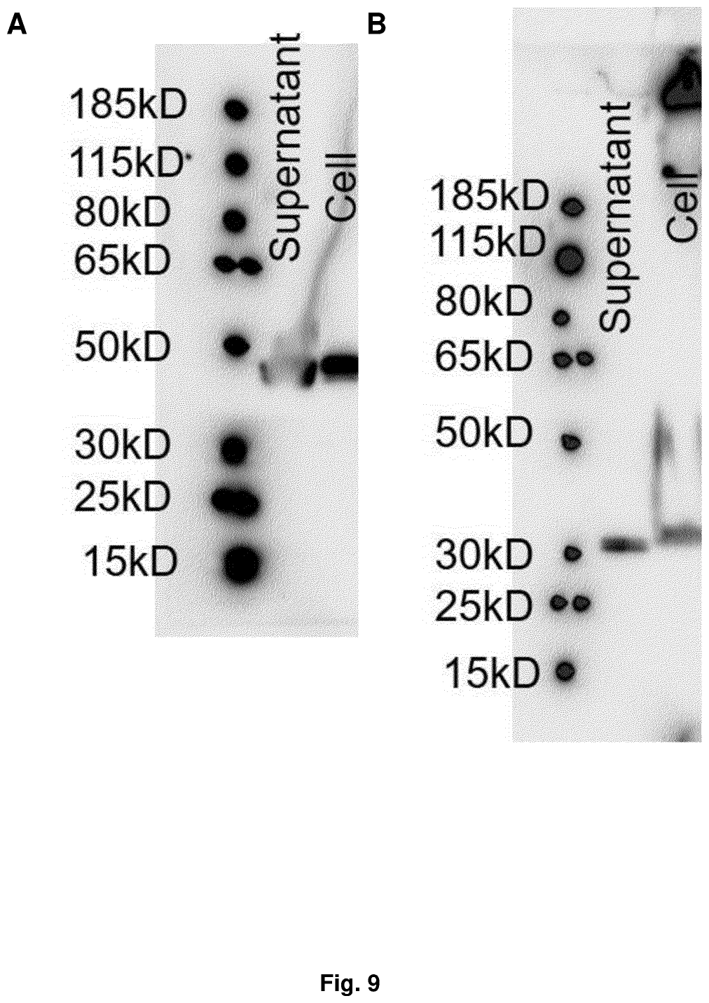

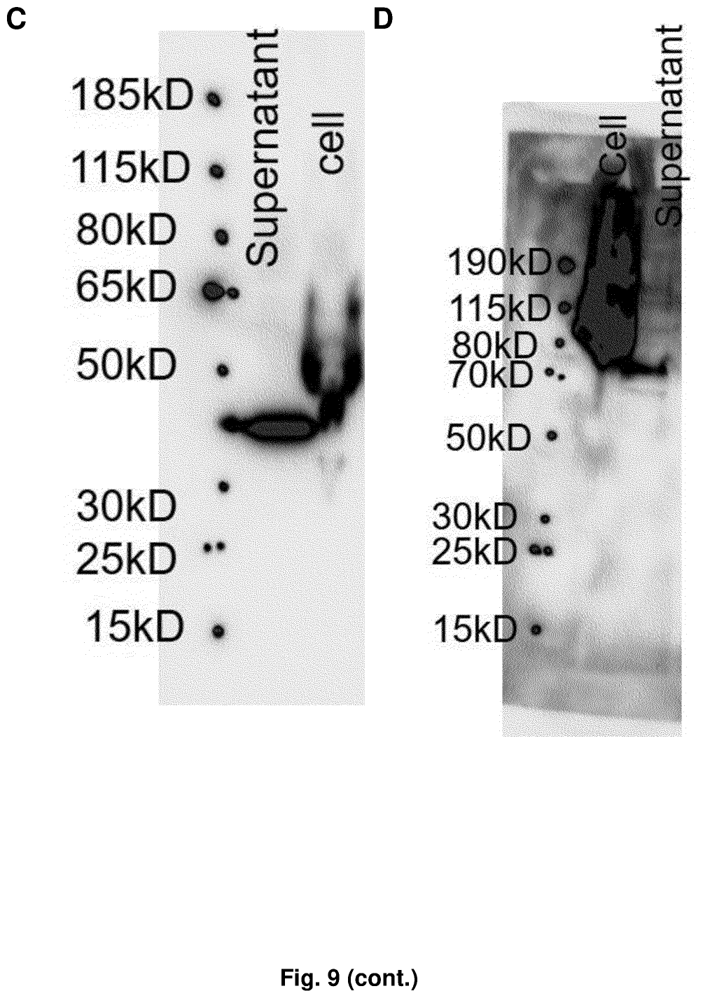

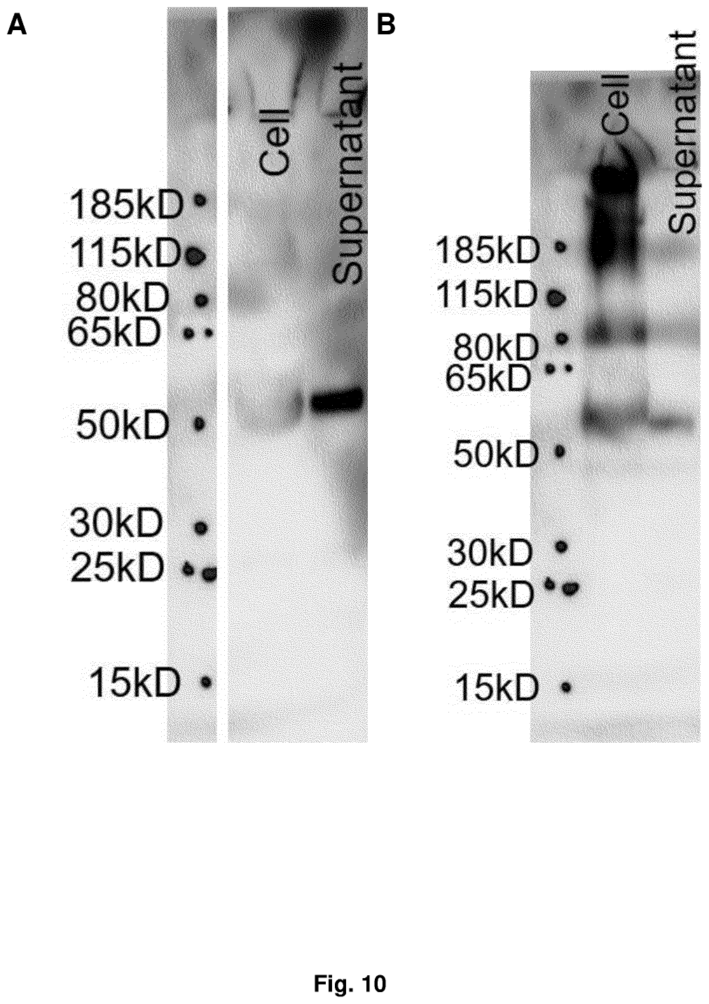

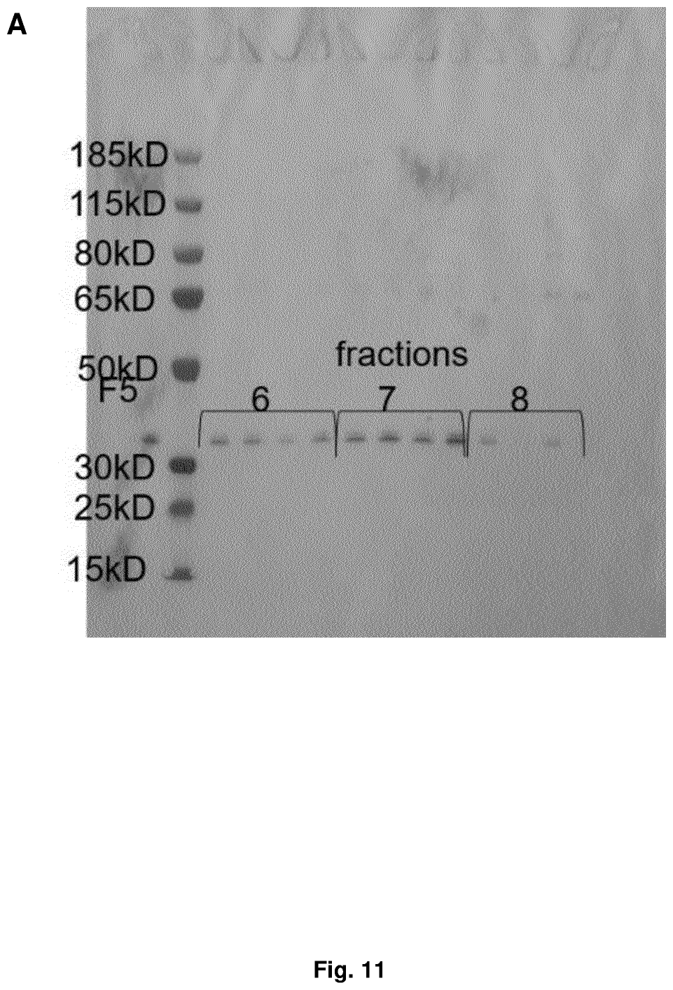

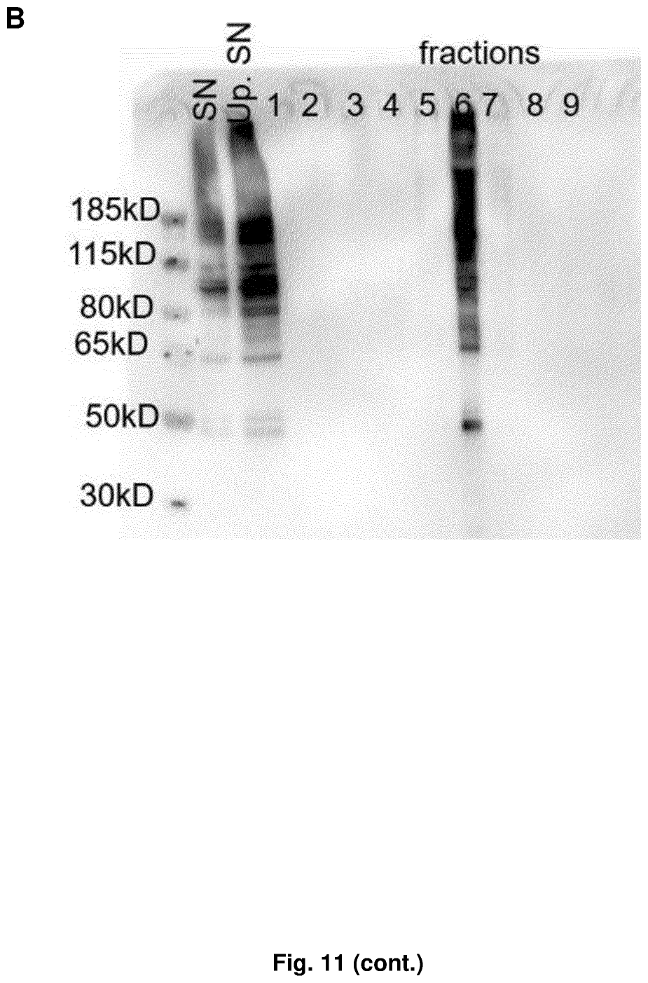

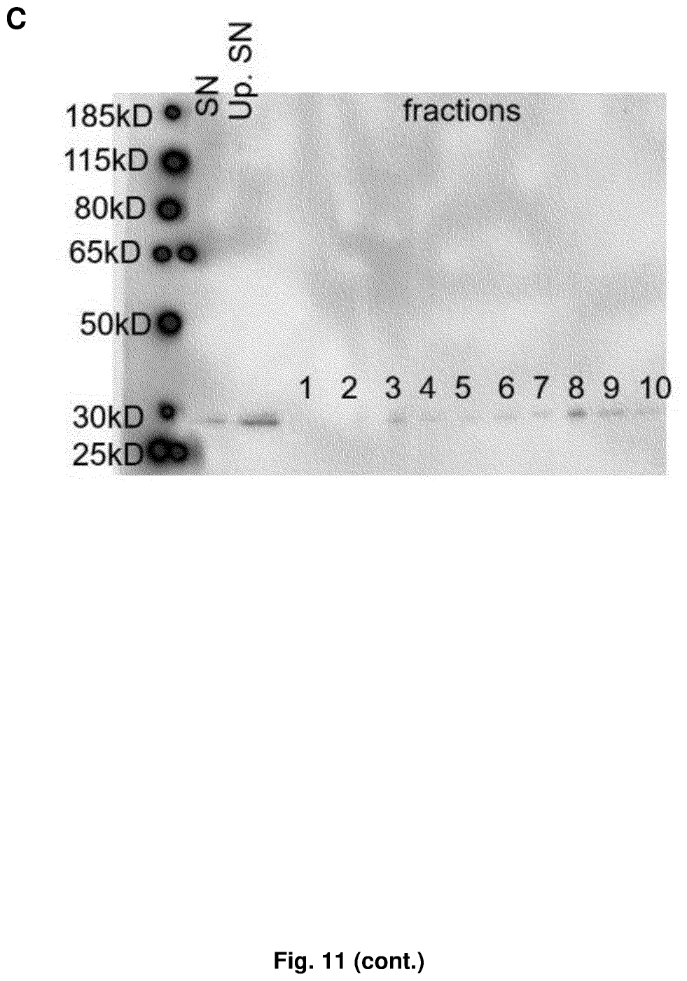

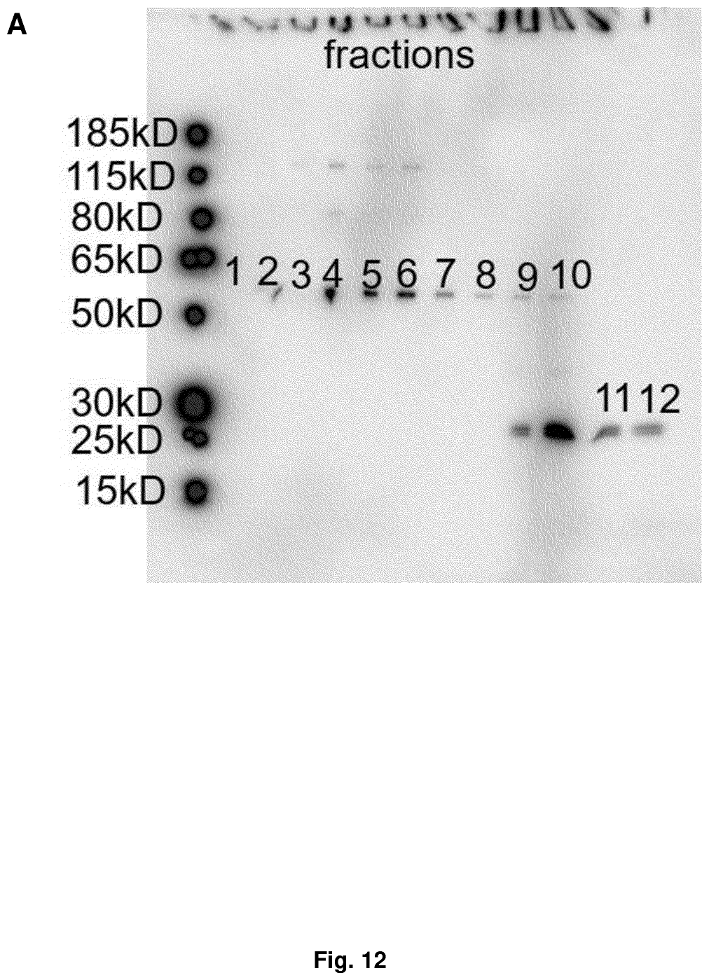

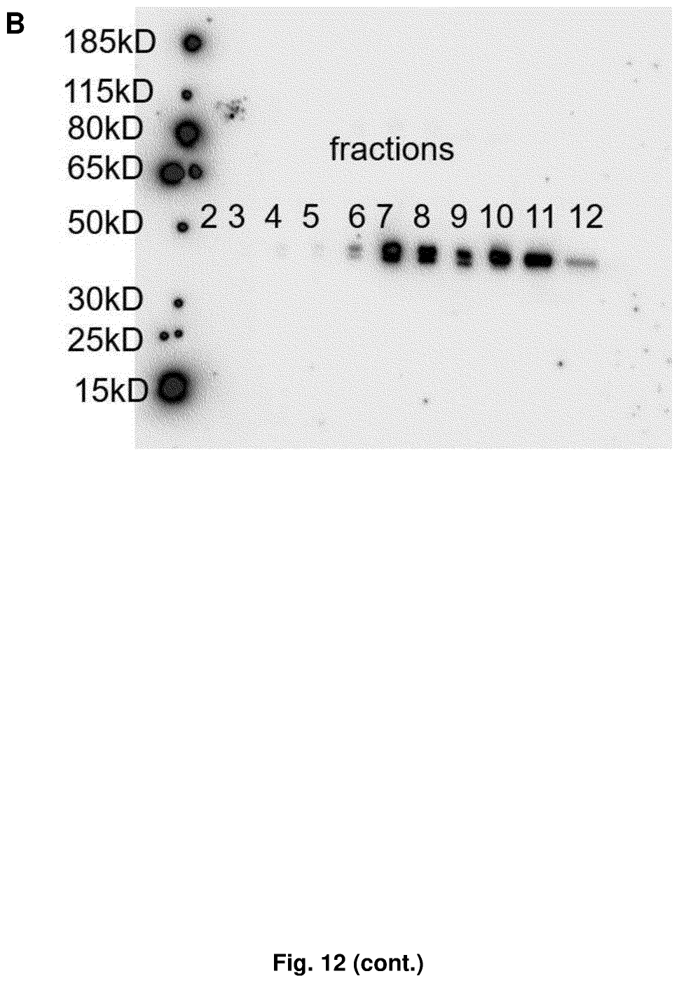

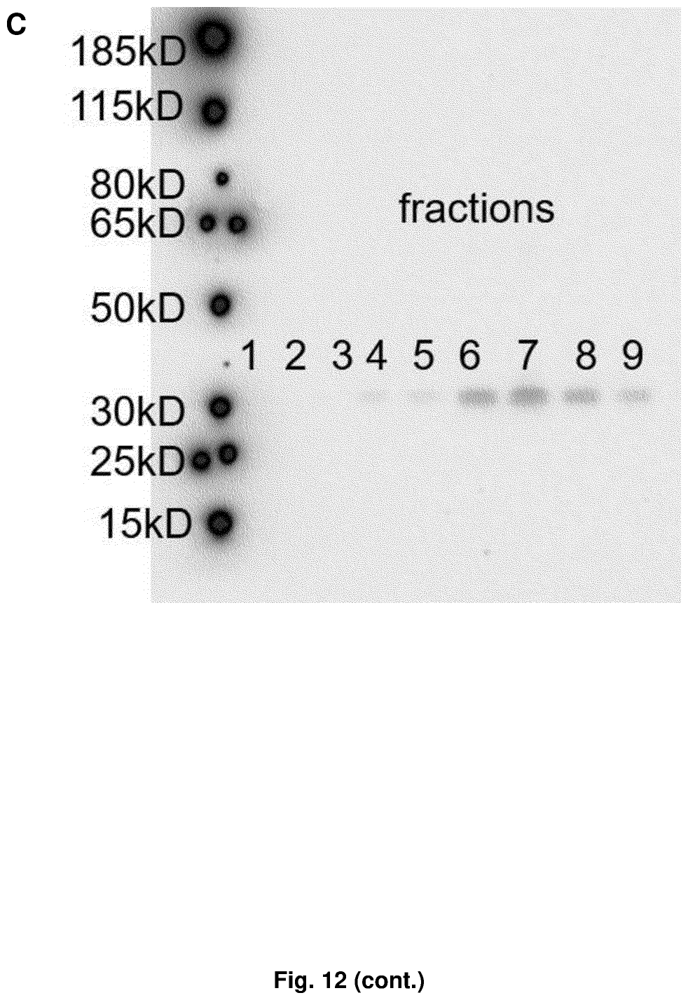

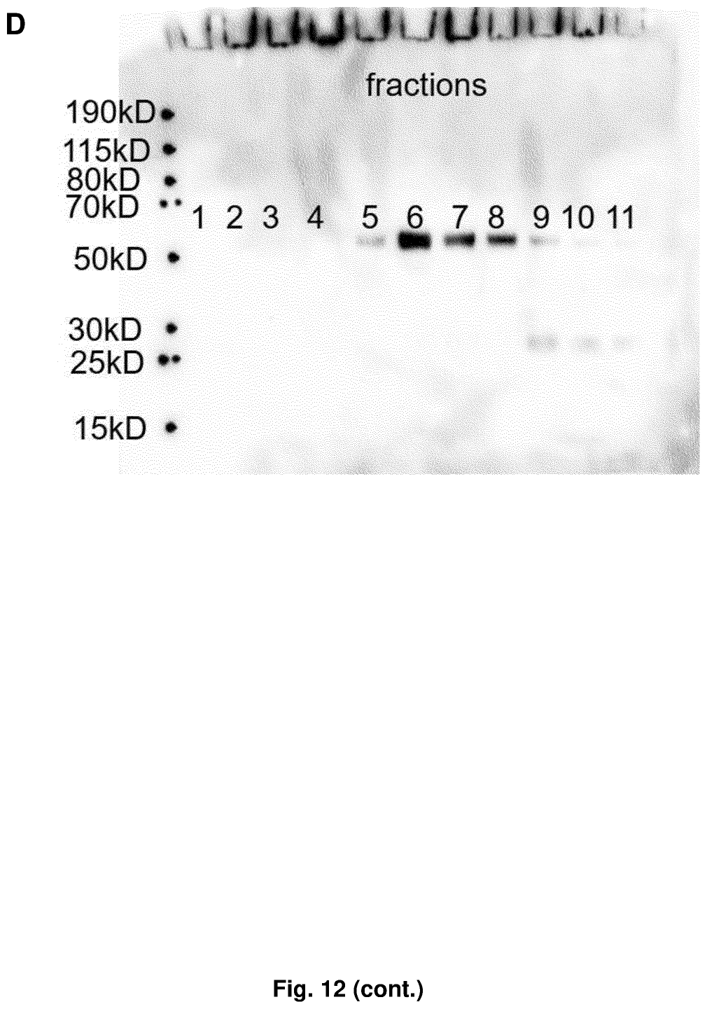

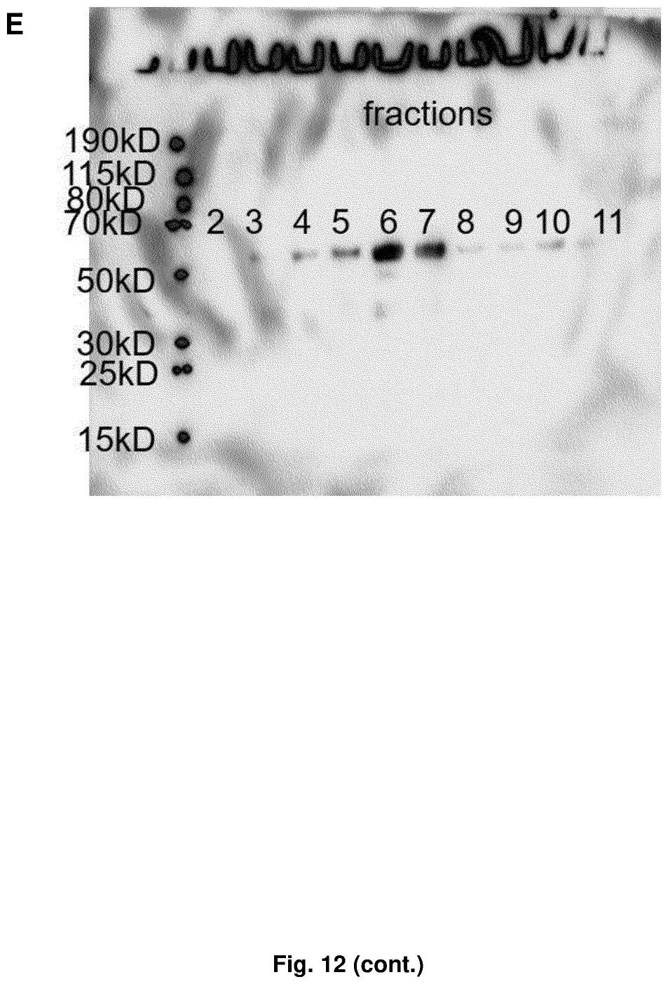

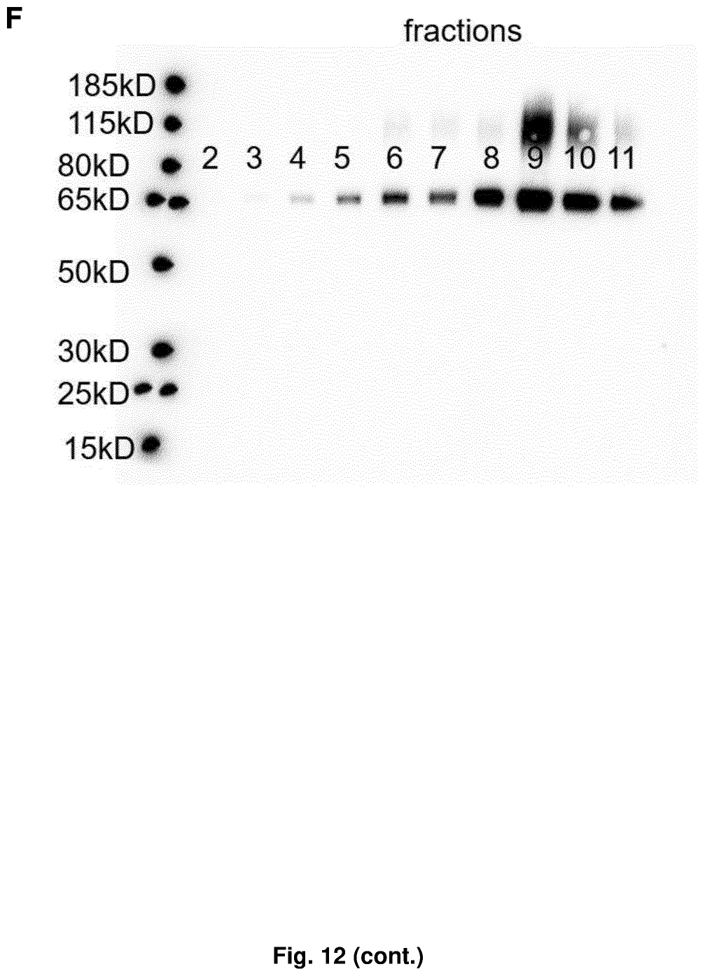

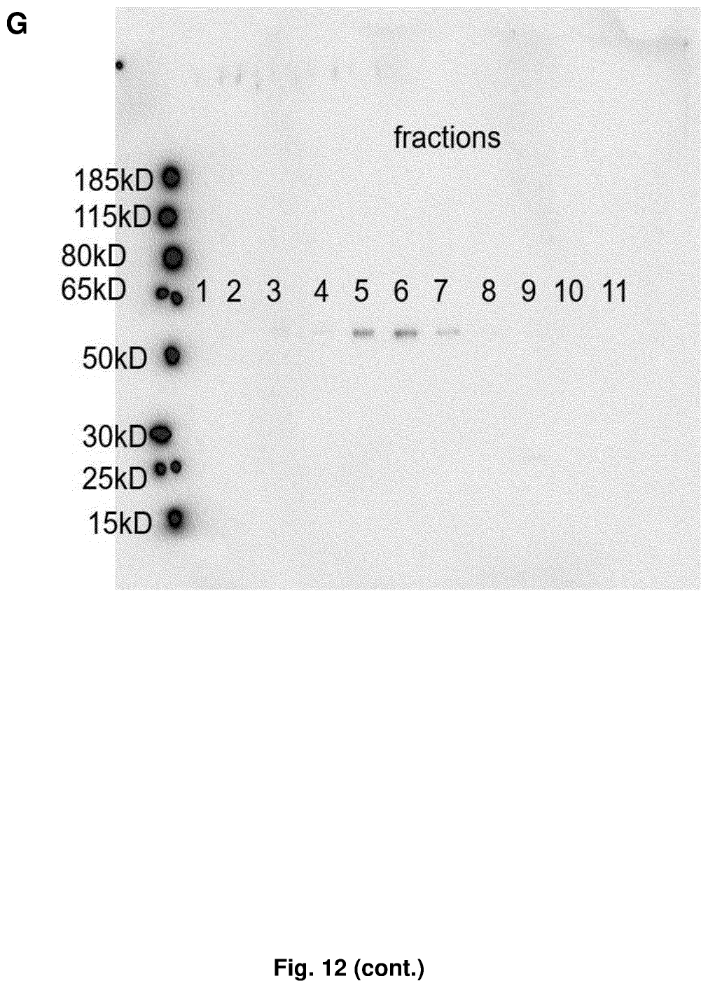

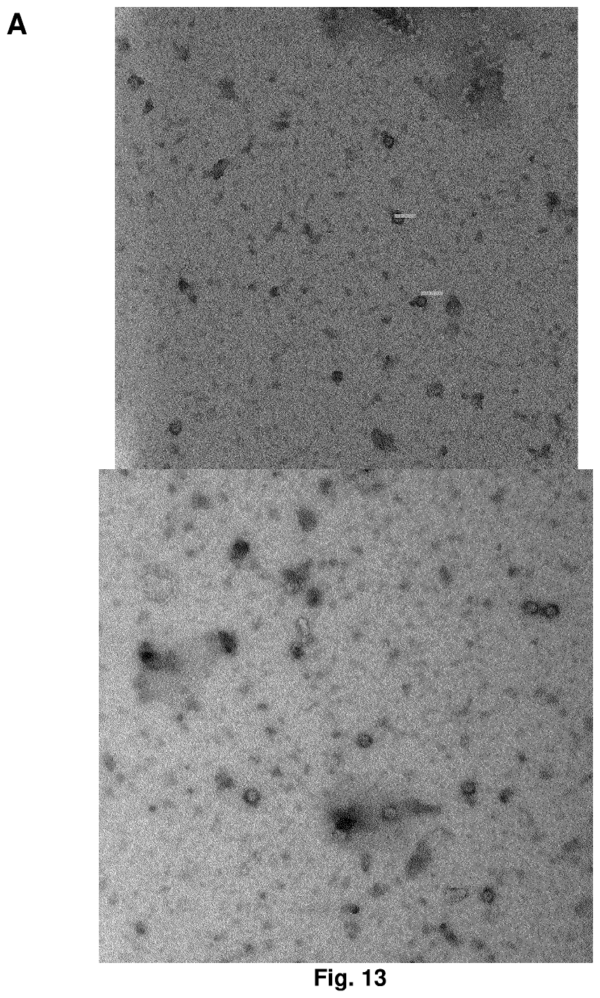

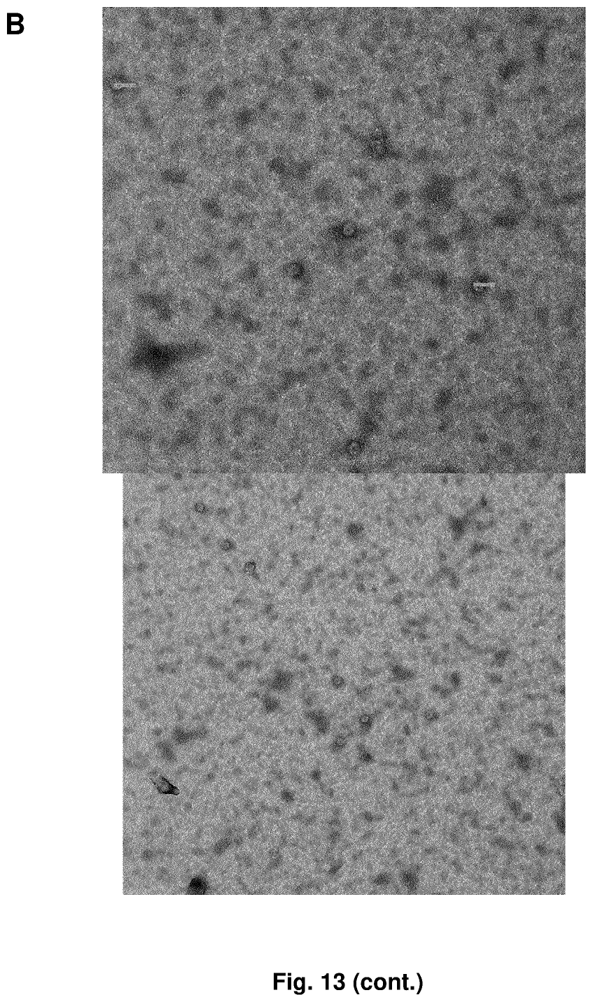

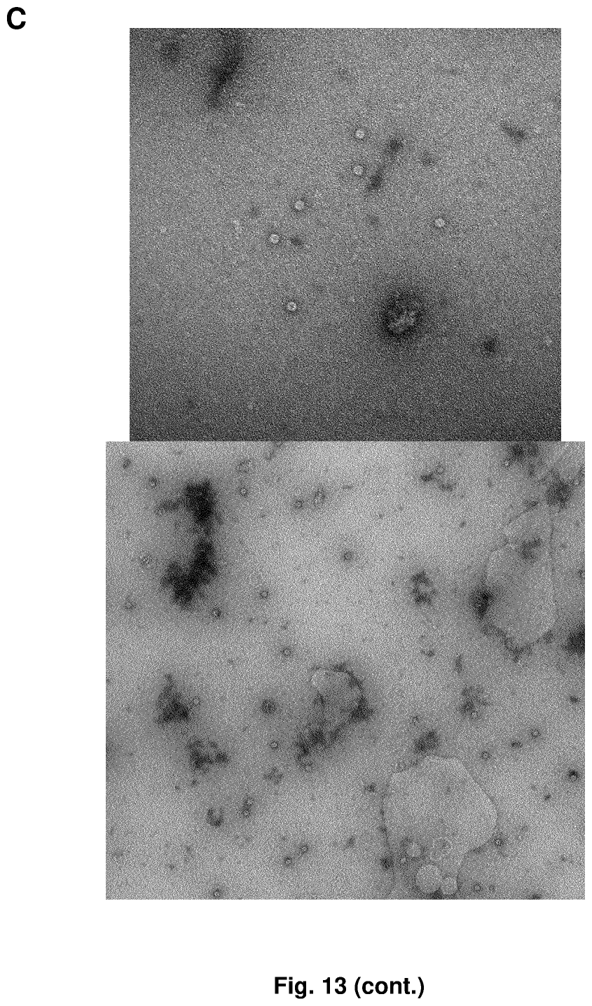

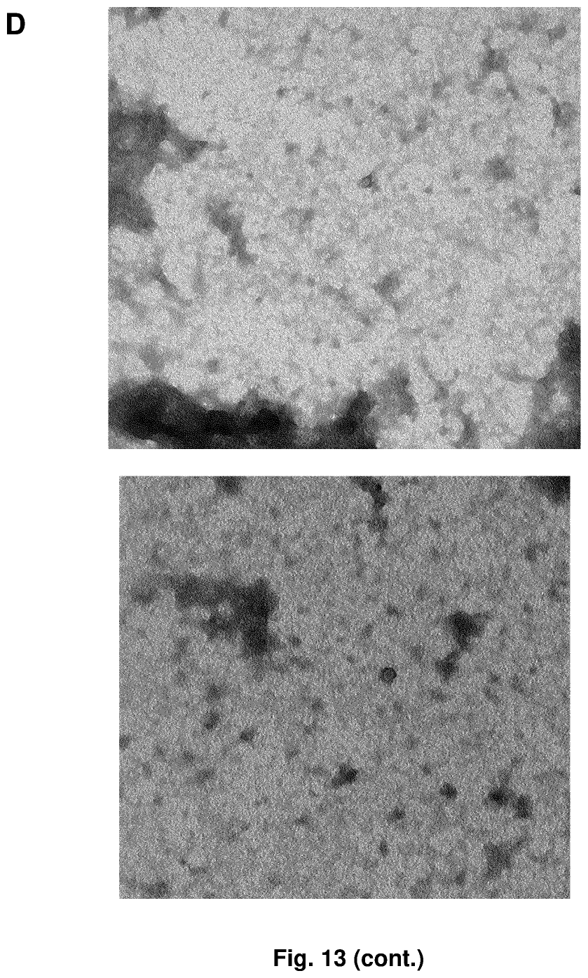

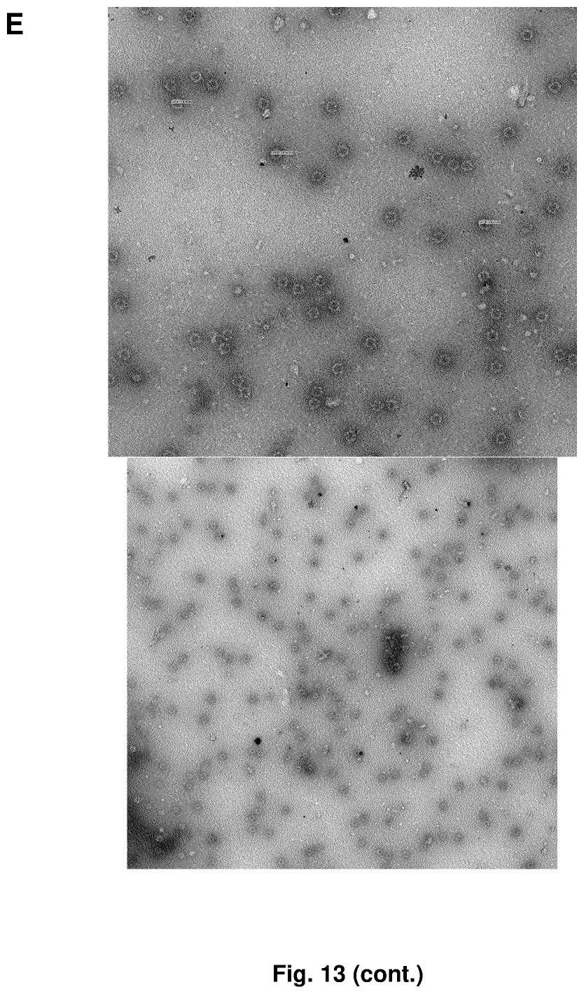

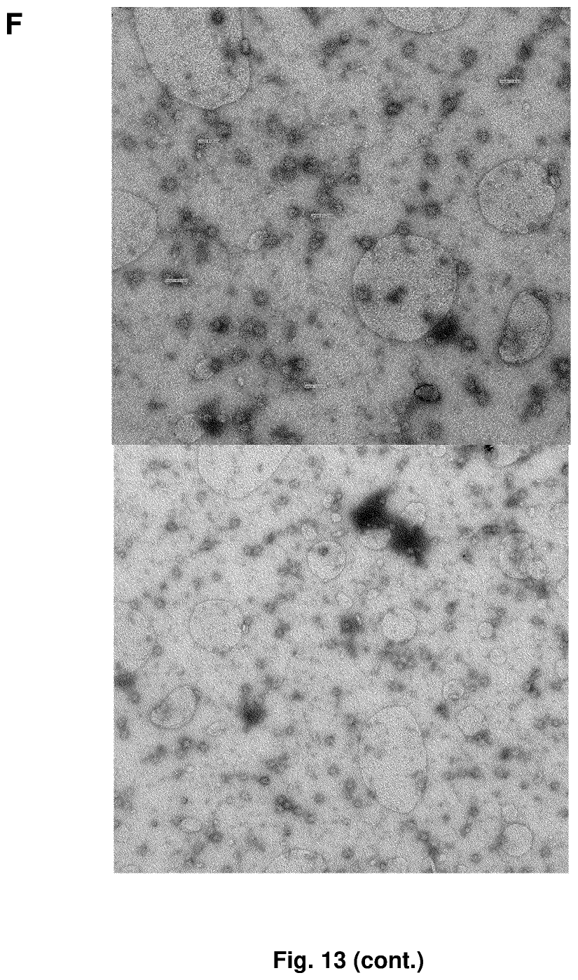

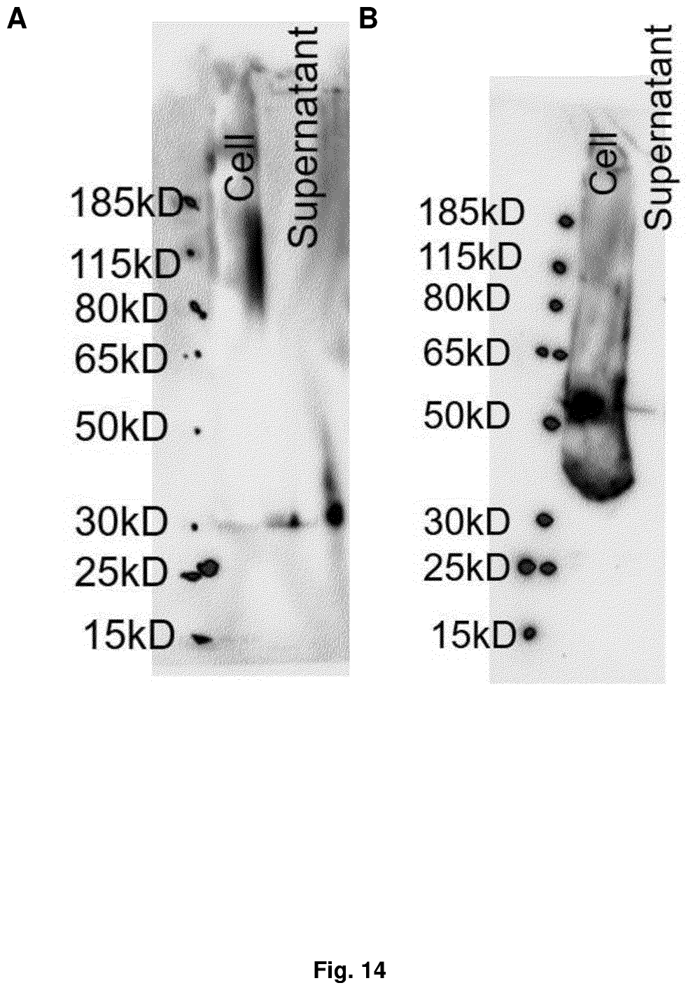

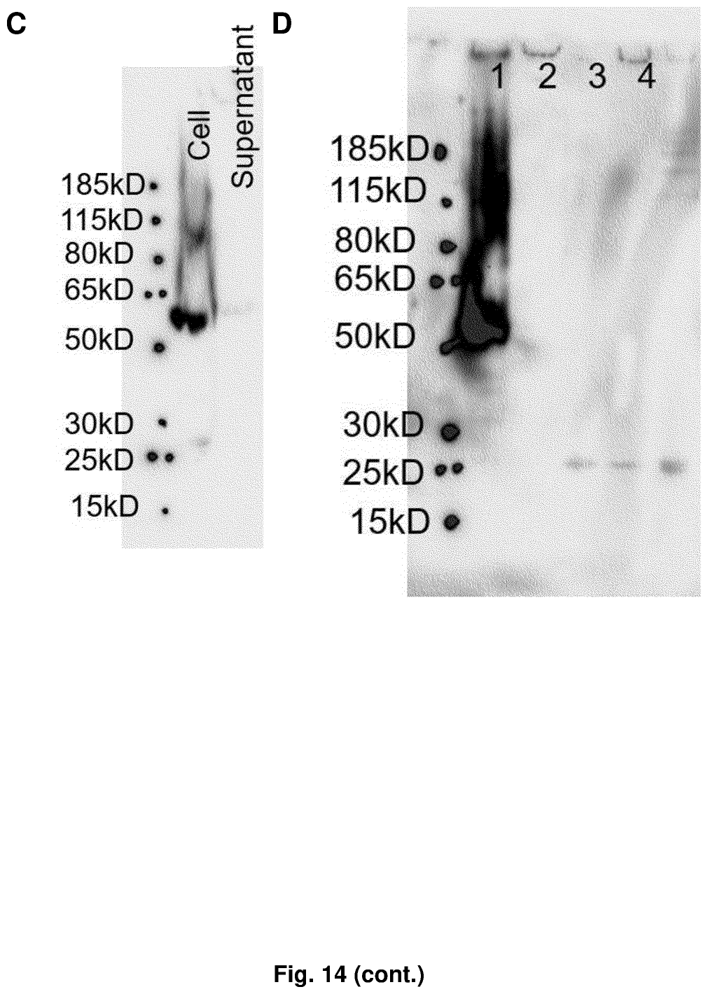

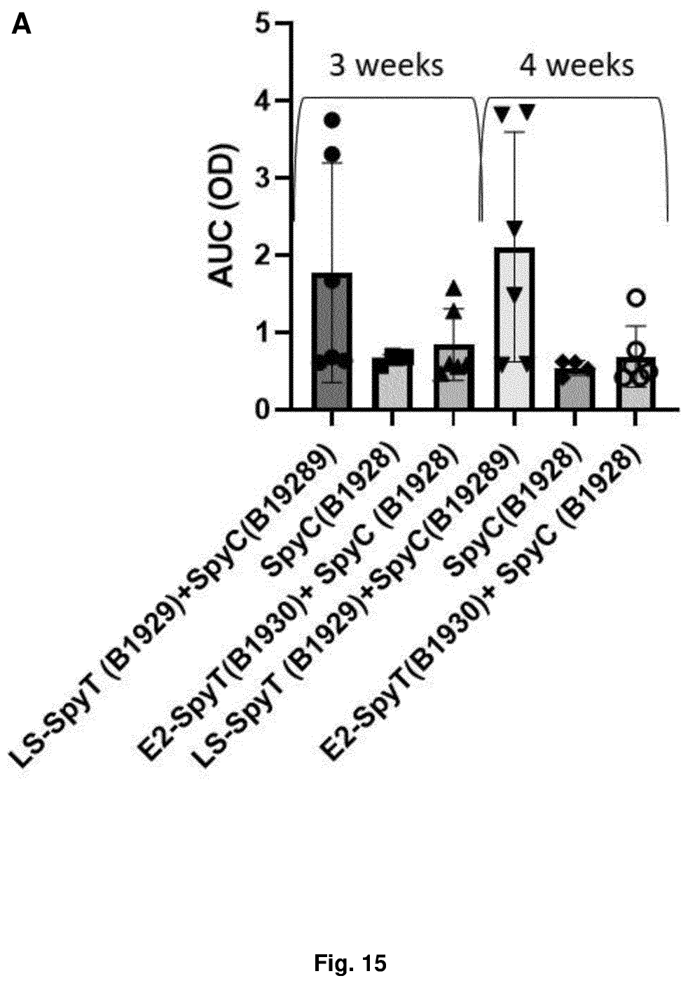

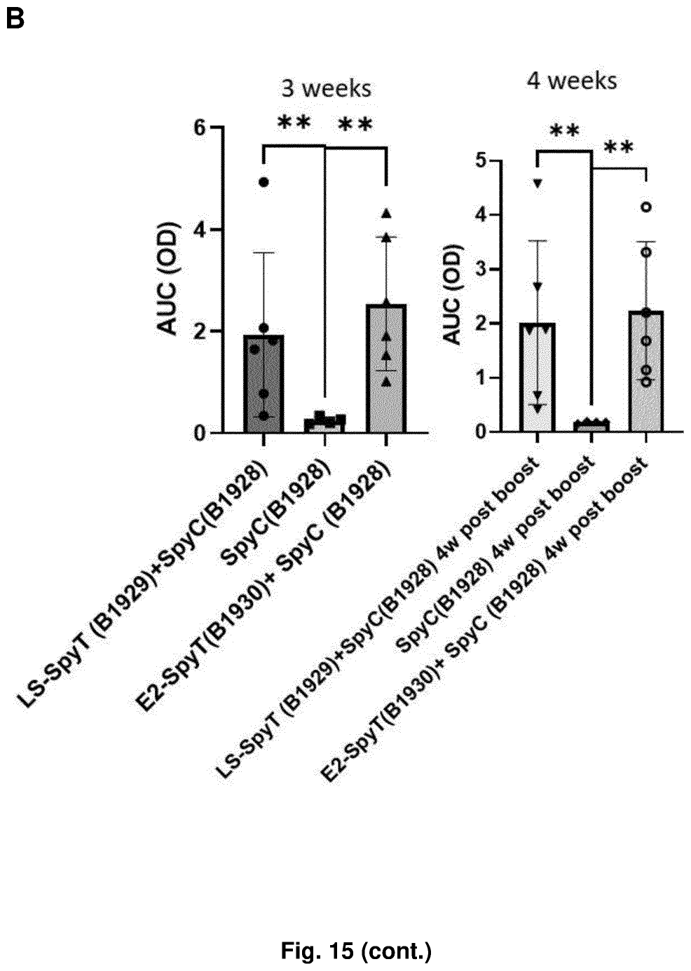

Modular combinatorial INFUSION shuffling and cloning of constructs into a plasmid vector suitable for DNA immunization (pVAX1—see FIG. 5) was performed.

Mouse derived C2C12 myoblast precursor cell line was transfected (reverse transfection, 2×24 well plates using Lipofectamine 2000 reagent) with:
A) SPYCEGFP (n=3)
B) SPYCEGFP and SpyTAP205 (n=3)
C) SPYCEGFP and HBcSpyT (n=3)

### Example 2—Formation of Distinct Intracellular Particles in Transfected Mammalian Cells

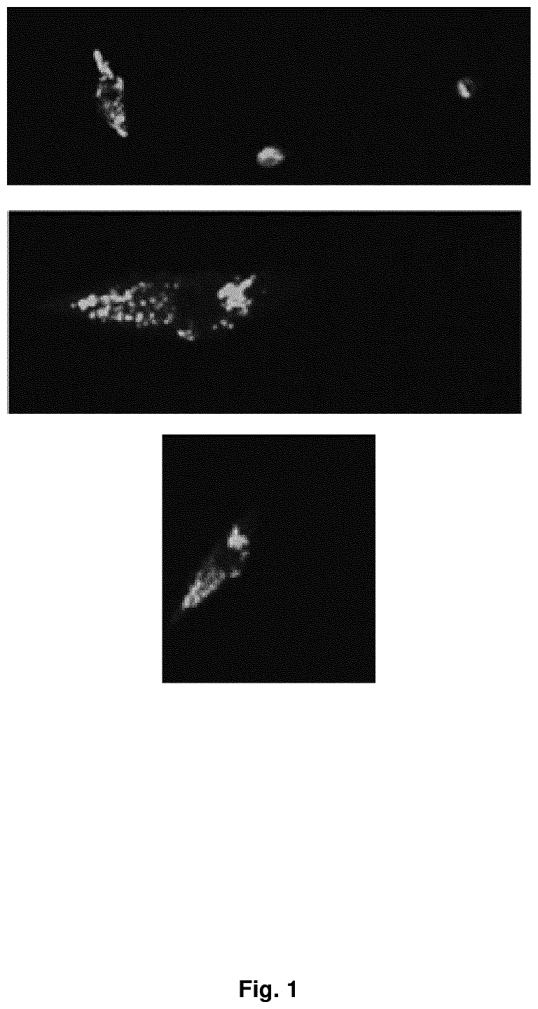

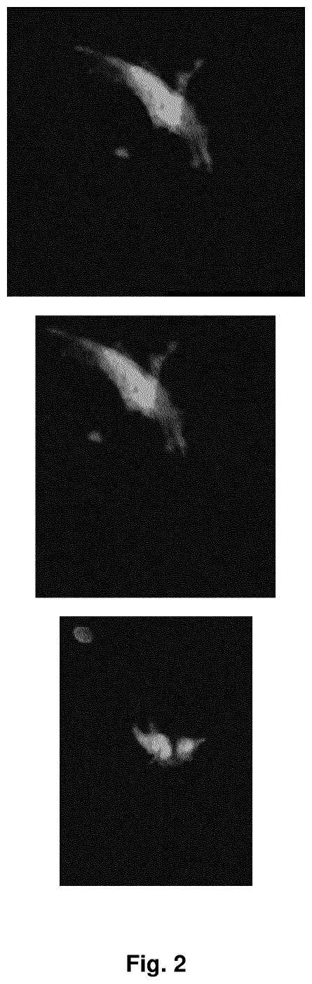

Co-transfection of eukaryotic cells with eGFP (genetically fused at N-terminus with SpyCatcher) and the Hepatitis B core antigen (genetically fused to SpyTag at C-terminus) resulted in the apparent expression of both components, which subsequently were able to bind (via interaction between SpyTag and SpyCatcher) to each other and form particulate complexes displaying eGFP. Specifically, confocal laser scanning microscopy (CLSM) of mouse cells harvested 72 hours after co-transfected with SpyCatcher-eGFP and Spytagged HBcore, respectively, revealed a distinct peri-nuclear distribution of eGFP fluorescent signals, indicating particularization of eGFP (see FIG. 1).

In contrast, transfection with SpyCatcher-eGFP and a Spytagged AP205 coat protein (previously shown to spontaneously form VLP upon expression in E. coli) resulted a diffuse/smeared eGFP fluorescent signal throughout the cell (see FIG. 2).

Overall, these results indicate that both the SpyCatcher eGFP and HBcSpyT protein were successfully expressed and that the eGFP was able to bind (via spyTag/SpyCatcher) to particulate structures formed by the HBcSpyT capsid protein.

### Example 3—Simultaneous Expression and Assembly of Encoded Proteins into Particulate Complexes in Mammalian Cells

To further investigate if the Spytagged coat proteins (AP205 and HBcore) could be expressed and form virus-like particles, and whether the SpyCatcher-eGFP protein could also bind (via spyTag/SpyCatcher) to such particles, we harvested the co-transfected C2C12 cells (48 hours after transfection), which were then sonicated for 45 s (3 times, 30%) and fractionated by ultracentrifugation (UC) using an iodixanol ultracentrifugation density gradient column. The post-UC fractions (incl. controls) were run on SDS-PAGE and transferred to a nitrocellulose membrane, which was finally probed with polyclonal HRP conjugated anti-GFP antibodies.

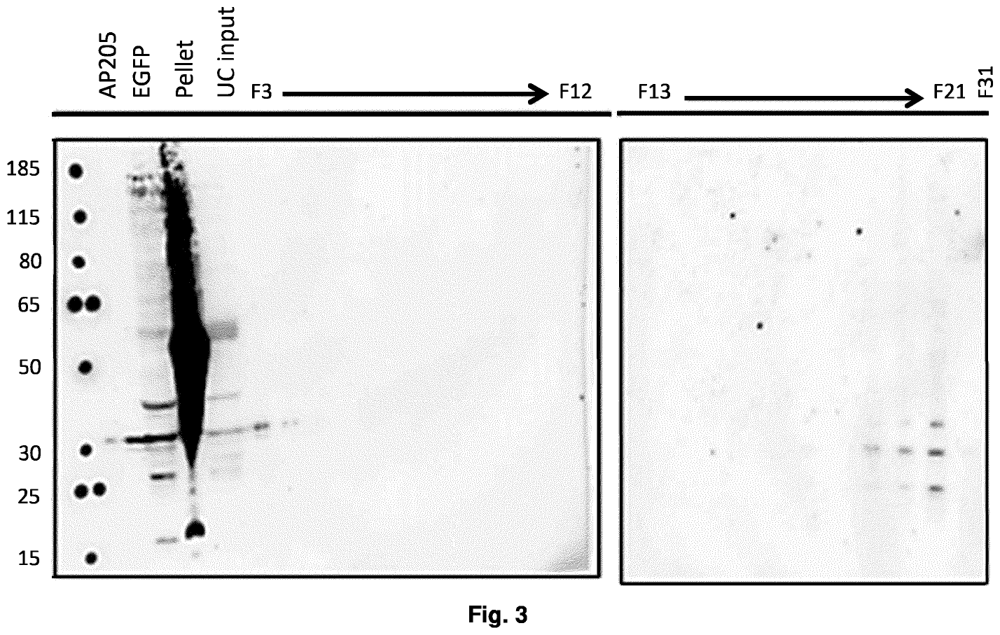

This experiment indicated that co-transfection of SpyCatcher eGFP and SpyTagged AP205 may have led to expression and conjugation of the two proteins, as indicated by the appearance of a protein band in the UC input lane (/pellet lane) having the approximate size of the theoretical size (i.e. 59 kDA) of the conjugate. However, there were not seen any protein bands of that size in any of the UC fractions (F3-F21), which could have contained particulate proteins (FIG. 3).

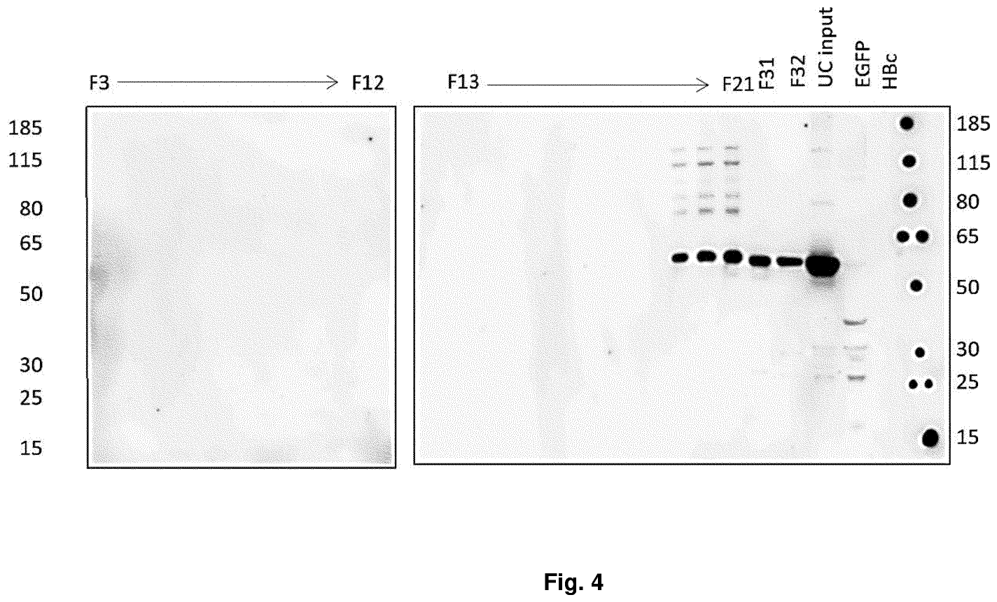

However, a similar analysis of C2C12 cells co-transfected with SpyT-HBc and SpyCatcher-eGFP, shows a clear protein band of apprx. 64 kDa (i.e. the theoretical size of a SpyTHBc:SpyCatcher-eGFP conjugate) both in the UC-input sample as well as in post UC fractions (F3-21, F31 and F32) including in high-density fractions expected to contain particulate protein complexes (FIG. 4). These results further indicate that co-transfection of Spytagged HBcore antigen and SpyCatcher-eGFP is able to simultaneously express and assemble into particulate complexes in a mammalian cell.

### Example 4—DNA Sequences Used in Example 5 to 10

The following nomenclature is used throughout examples 5 to 10 to refer to DNA sequences encoding the indicated amino acid SEQ ID NO:s.

| | | Encodes |
|---|---|---|
| Abbre- | | SEQ |
| viation | Description | ID NO: |
| Sign1 | Secretion signal tag (from azurocidin) | 79 |
| Sign2 | Secretion signal tag (from serum albumin) | 80 |
| Sign3 | Secretion signal tag (from modified serum | 81 |
| | albumin) | |
| Sign4 | Secretion signal tag (from Ig kappa chain V III | 82 |
| | region) | |
| Sign5 | Secretion signal tag (from Modified Ig kappa | 83 |
| | chain V III region MOPC 63 like (mIgκ C)) | |
| Sign6 | Secretion signal tag (from Modified Ig kappa | 84 |
| | chain V III region VG (mIgκ H)) | |
| Sign7 | Secretion signal tag (from tissue plasmogen | 99 |
| | activator) | |
| Sign8 | Secretion signal tag (from chymotrypsinogen B) | 100 |
| Sign9 | Secretion signal tag (from secreted embryonic | 101 |
| | alkaline phosphatase) | |
| SpyC | SpyCatcher | 21 |
| i301 | i301 particle-forming protein | 72 |
| ctag | C-terminal tag | 91 |
| Hbc | Hepatitis core protein | 92 |
| tandemHBc | Tandem hepatitis core protein construct | 93 |
| SpyT | SpyTag | 1 |
| Ferritin | Human ferritin (amino acids 5-174) | 69 |
| eGFP | Enhanced green fluorescent protein | 94 |
| His | His purification tag | 95 |
| Pfs25 | Plasmodium falciparum antigen | 96 |
| E2 | 2-oxo acid dehydrogenase subunit E2 | 97 |
| LS | Lumazine synthase | 70 |
| Norovirus | Norovirus capsid protein | 98 |

### Example 5—Expression of Soluble Antigens and Particle-Forming Proteins from Plasmid DNA

### Methods

DNA was cloned in pVAX1 vector (V26020, thermoFisher) and cloned in E. coli (One shot Top10, Invitrogen, C404006). Vectors were purified using a midiprep kit.

HEK293-Freestyle cells were transfected or co-transfected with 37.5 ug in 30 mL culture using FreeStyle™ MAX Reagent (16447100, Life Technologies).

After incubation for 6 days, cells and supernatant were harvested. Supernatant was frozen down at −20° C. and represent the samples called supernatant (SN). Cells were pelleted by spinning down at 1200 RPM, 5 min, and resuspended in PBS with Complete™, Mini, EDTA-free Protease Inhibitor Cocktail (Sigma, 11836170001). Cells were sonicated at 30%, 45 s, 3 times and spun down 20.000 g, 10 min, 4° C. The supernatant is frozen at −20° C. and represent the samples called “cell”.

Both cell and SN were run on denaturing SDS+DTT gels (15 uL loaded), the SDS gel was then transferred on a Nitrocellulose membrane and blocked in TBS-T 5% milk overnight. Proteins on the membrane were detected with a primary antibody (as specified for each gel) and if necessary a secondary antibody (as specified for each gel). Membrane was washed 3 times between each antibody and prior to development with TBS-T. The membrane was developed with ECL substrate according to manufacturer's instructions.

### Results and Conclusion

After DNA transfection in mammalian cells in vitro, expression and secretion (through a secretion signal) of particles and soluble antigens could be detected by WB of the cell and SN from transfected cells. The Western blots shows the appearance of a band at the expected size for the particle or soluble antigen.

This was shown for the following:
Sign3-SpyC-i301-ctag
Sign8-tandemHBc-SpyC
Sign8-HBc-SpyT
Sign9-Ferritin-SpyC
Sign8-SpyT-eGFP
Sign8-eGFP
Sign8-SpyC-His
Sign7-Pfs25-SpyT-Ctag
Sign8-SpyC-eGFP

As seen in FIGS. 6 and 7, bands of the expected size in cell and supernatant samples for all constructs were observed, thus indicating expression and secretion of the particle-forming subunit proteins as well as soluble protein antigens in human (HEK) cells, after plasmid DNA transfection.

### Example 6—Coupling of Particles and Proteins Expressed from Plasmid DNA

### Methods

HEK293-Freestyle cells were co-transfected with 37.5 ug (18.75 ug of each vector) in 30 mL culture using FreeStyle™ MAX Reagent (16447100, Life Technologies).

Cells were co-transfected with a vector encoding for a particle and a vector encoding for a protein with a compatible tag-catcher for coupling.

After incubation for 6 days, cells and supernatant were harvested. Supernatant was frozen down at −20° C. and represent the samples called supernatant (SN). Cells were pelleted by spinning down at 1200 RPM, 5 min, and resuspended in PBS with Complete™, Mini, EDTA-free Protease Inhibitor Cocktail (Sigma, 11836170001). Cells were sonicated at 30%, 45 s, 3 times and spun down 20 000 g, 10 min, 4° C. The supernatant is frozen at −20° C. and represent the samples called “cell”.

Both cell and SN were run on denaturing SDS+DTT gels (15 uL loaded), the SDS gel was then transferred on a Nitrocellulose membrane and blocked in TBS-T 5% milk overnight. Proteins on the membrane were detected with a primary antibody (as specified for each gel) and if necessary a secondary antibody (as specified for each gel). Membrane was washed 3 times between each antibody and prior to development with TBS-T. The membrane was developed with ECL substrate according to the manufacturer's instructions.

### Results and Conclusion

After in vitro DNA co-transfection of mammalian (HEK293) cells, the isopeptide bond-mediated conjugation of individually expressed antigens and particle-forming proteins, respectively, could be verified by WB by detecting a band of the expected size of the conjugated antigen and particle-forming protein.

This was shown for the following:
Sign8-SpyT-eGFP and sign9-SpyC-Ferritin
Sign8-SpyC-eGFP and sign8-Hbc-SpyT
Sign8-tandemHBc-SpyC and sign8-SpyT-eGFP
Sign3-SpyC-i301-ctag and sign8-SpyT-eGFP
Sign9-SpyT-E2 and sign8-SpyC-eGFP
Sign9-SpyT-LS and sign8-SpyC-eGFP
Sign8-SpyC-His and sign9-SpyT-E2
Sign8-SpyC-His and sign9-LS-SpyT
Sign8-SpyC-His and sign8-Hbc-SpyT
Sign8-SpyC-Ferritin and sign7-Pfs25-SpyT
Sign3-SpyC-i301-Ctag and sign7-Pfs25-SpyT
Sign8-Norovirus-SpyT and sign8-SpyC-His

As seen in FIGS. 8, 9 and 10, bands of the expected size for coupled eGFP, SpyCatcher and Pfs25 (model antigens) to different particle-forming subunit proteins in cells and supernatant for all constructs were observed. This indicates that the antigens and the particles with the corresponding tag/catcher are able to couple in vitro, after co-transfection in human (HEK) cells.

### Example 7—Verification of Nanoparticle Formation by Ultracentrifugation and Western Blotting

### Methods

After HEK cell transfection and harvest, supernatant was loaded onto an Optiprep step gradient (23, 29 and 35%) followed by centrifuged for 3 h 30 min at 47800 g, 16° C. The gradient was then dripped into fractions (F1-F12) each fraction containing approximately 250 uL.

If a particle was formed it is expected that it will be found in the middle fractions (F3-F8).

All fractions were run on denaturing SDS+DTT gels (15 μL loaded), the SDS gel was then transferred on a Nitrocellulose membrane and blocked in TBS-T 5% milk overnight. Proteins on the membrane were detected with a primary antibody (as specified for each gel) and if necessary a secondary antibody (as specified for each gel). The membrane was washed 3 times between each antibody and prior to development with TBS-T. The membrane was developed with ECL substrate according to the manufacturer's instructions.

### Results and Conclusion

After DNA co-transfection or transfection in mammalian cells in vitro, we could detect potential particle formation and secretion. Particle formation can be expected when the proteins are found within the fractions 3-8 from the density gradient. This was the case for the following:
Sign3-SpyC-i301-Ctag
Sign8-tandemHBc-SpyCatcher
Sign9-Ferritin-SpyC
Sign8-SpyT-eGFP+sign9-SpyC-Ferritin
Sign8-SpyC-His+sign9-SpyT-E2
Sign8-SpyC-His+sign9-LS-SpyT
Sign9-SpyC-Ferritin+sign7-Pfs25-SpyT
Sign3-SpyC-i301+sign7-Pfs25-SpyT
Sign9-SpyT-E2+sign8-SpyC-eGFP
Sign9-LS-SpyT+sign8-SpyC-eGFP

As seen in FIGS. 11 and 12, particle formation as well as the formation of particles coupled to antigens were observed in all depicted constructs, as bands are seen of the expected sizes present in the relevant fractions (fractions 3-8). This thus indicates that after transfection with plasmid DNA in human (HEK) cells, there was not only expression and secretion of the particle subunits and the soluble antigens in vivo, but also formation of the particles and of the particles coupled to antigens in vitro.

### Example 8—Visualisation of Particle Formation by Transmission Electronic Microscopy

After HEK cell transfection, harvest and purification by ultracentrifugation, samples were run on transmission electronic microscopy (TEM).

After DNA co-transfection or transfection in mammalian cells in vitro, secreted particle formation was visualized by TEM.

This was shown for the following:
Sign9-Ferritin-SpyC
Sign8-SpyC-His+sign9-LS-SpyT
Sign9-SpyT-E2+sign8-SpyC-His
Sign8-SpyT-eGFP+sign9-SpyC-Ferritin
Sign3-SpyC-i301-Ctag+sign7-Pfs25-SpyT-Ctag
Sign9-SpyC-Ferritin+sign7-Pfs25-SpyT-Ctag

As can be seen from FIG. 13, particles of the expected size formed in the supernatant of transfected or co-transfected cells. Thus, it is further confirmed that particles and coupled particles are able to form in the supernatant of transfected or co-transfected cells in vitro from plasmid DNA.

### Example 9—Verification of Intracellular Conjugation of the Antigen to the Nanoparticle-Forming Protein

### Methods

DNA was cloned in pVAX1 (V26020, thermoFisher) vector and cloned in E. coli (One shot Top10, Invitrogen, C404006). Vectors were purified using a midiprep kit.

HEK293-Freestyle cells were transfected or co-transfected with 37.5 ug in 30 mL culture using FreeStyle™ MAX Reagent (16447100, Life Technologies).

After incubation for 6 days, cells and supernatant were harvested. Supernatant was frozen down at −20° C. and represent the samples called supernatant (SN). Cells were pelleted by spinning down at 1200 RPM, 5 min, and resuspended in 1×SDS+DTT and frozen at −20° C. and represent the samples called “cell”.

Both cell and SN were run on denaturing SDS+DTT gels (15 μL loaded), the SDS gel was then transferred on a Nitrocellulose membrane and blocked in TBS-T 5% milk overnight. Proteins on the membrane were detected with a primary antibody (as specified for each gel) and if necessary a secondary antibody (as specified for each gel). Membrane was washed 3 times between each antibody and prior to development with TBS-T. Membrane was developed with ECL substrate according to manufacturer's instructions.

### Results and Conclusion

This technique allows for visualization of coupling between the particle and the soluble antigen in the cells. We have previously shown that some particles could couple with some antigens, however we did not know if it was happening after secretion or already inside the cell. This experiment shows that coupling is happening intracellularly, as we observe the coupling band from the cell substrate harvested in SDS+DTT (these compounds prohibiting coupling after harvest).

This was shown for the following:
Sign8-SpyC-His and sign9-LS-SpyT
Sign8-SpyT-eGFP+sign9-SpyC-Ferritin
Sign3-SpyC-i301-ctag and sign8-SpyT-eGFP
Sign8-eGFP-SpyC and sign9-LS-SpyT

As seen in FIG. 14, particles and soluble antigens were able to couple intracellularly, and not only in the supernatant. Indeed, a band was visualised of the expected coupling size in cells harvested with SDS+DTT, thus indicating that the coupling occurred inside the cells in the in vitro culture.

### Example 10—Plasmid DNA Immunization in Mice

### Methods

DNA encoding for particles and/or soluble antigen was used for vaccination in mice. Balb/c mice were immunized with 30 μg of LS-SpyT and 30 μg of SpyC (N=6), or 30 μg of SpyC (N=4), or 30 μg of E2-SpyT and 30 μg of SpyC (N=6). DNA was formulated in PBS, and injected in the right thigh muscle.

Mice were immunized on day 0 and week 5, and blood was drawn on week 3 and 4 post prime and post boost. Serum was isolated from the blood and run on ELISA for detection of anti-SpyC IgG. For that purpose, 96-well plates (Nunc MaxiSorp) were coated overnight at 4° C. with 0.1 μg/well SpyC in PBS. Plates were blocked for 1 hour at room temperature (RT) using 0.5% skimmed milk in PBS. Mouse serum was diluted 1:50 or 1:10 in blocking buffer, and added to the plate in a 2-fold dilution, followed by incubation for 1 hour at RT. Plates were washed three times in PBS in between steps. In order to measure total serum IgG, Horseradish peroxidase (HRP) conjugated goat anti-mouse IgG (Life technologies, A16072) was diluted 1:1000 in blocking buffer followed by 1 hour incubation at RT. Plates were developed with TMB X-tra substrate (Kem-En-Tec, 4800A) and absorbance was measured at 450 nm. Data were collected on a BioSan HiPo MPP-96 microplate reader and analyzed using GraphPad Prism (San Diego, USA, version 8.4.3).

### Results and Conclusion

After DNA immunization in mice, it was seen that mice receiving DNA encoding the particles and DNA encoding for SpyC, have higher IgG titers against SpyC after only a first immunization, compared to mice receiving only the DNA encoding for the SpyC. Additionally, this trend is even higher after a boost immunization. This is true for both groups of mice that have received SpyC in combination with either the LS particle or the E2 particle.

This is shown in FIG. 15, where mice receiving DNA encoding for the particles and DNA encoding for SpyC, have higher IgG titers against SpyC after a first immunization than mice receiving only DNA encoding for SpyC, as shown by ELISA.

### Example 11—Expression of Soluble Antigens and Particle-Forming Proteins from mRNA

HEK293-Freestyle cells will be transfected or co-transfected with 37.5 μg in 30 mL culture using FreeStyle™ MAX Reagent (16447100, Life Technologies).

After incubation for 6 days, cells and supernatant will be harvested. Supernatant will frozen down at −20° C. and represent the samples called supernatant (SN). Cells will be pelleted by spinning down at 1200 RPM, 5 min, and resuspended in PBS with Complete™, Mini, EDTA-free Protease Inhibitor Cocktail (Sigma, 11836170001). Cells will be sonicated at 30%, 45 s, 3 times and spun down 20000 g, 10 min, 4° C. The supernatant is frozen at −20° C. and represent the samples called “cell”.

Both cell and SN will be run on denaturing SDS+DTT gels (15 μL loaded). The SDS gel will be transferred on a Nitrocellulose membrane and blocked in TBS-T 5% milk overnight. Proteins on the membrane will be detected with a primary antibody and if necessary a secondary antibody. Membrane will be washed 3 times between each antibody and prior to development with TBS-T. Membranes will be developed with ECL substrate according to the manufacturer's instructions.

After mRNA transfection in mammalian cells in vitro, expression and secretion (through a secretion signal) of particles and soluble antigens will thus be detected by Western blotting similar to what was described in Example 5.

Bands of the expected size in cells and supernatant for all constructs (particle-forming subunit proteins or soluble protein antigens), will indicate expression and secretion of soluble proteins in human (HEK) cells, after mRNA transfection.

### Example 12—Verification of the Conjugation of Soluble Antigen to Different Particle-Forming Proteins Expressed from mRNA

HEK293-Freestyle cells will be co-transfected with 37.5 μg (18.75 μg of each RNA) in 30 mL culture using FreeStyle™ MAX Reagent (16447100, Life Technologies)

Cells will be co-transfected with a vector encoding for a particle and a vector encoding for a protein with a compatible tag-catcher for coupling.

After incubation for 6 days, cells and supernatant will be harvested. Supernatant will be frozen down at −20° C. and represent the samples called supernatant (SN). Cells will be pelleted by spinning down at 1200 RPM, 5 min, and resuspended in PBS with Complete™, Mini, EDTA-free Protease Inhibitor Cocktail (Sigma, 11836170001). Cells will be sonicated at 30%, 45 s, 3 times and spun down 20000 g, 10 min, 4° C. The supernatant is frozen at −20° C. and represent the samples called “cell”.

Both cell and SN will be run on denaturing SDS+DTT gels (15 μL loaded). The SDS gel will be transferred on a Nitrocellulose membrane and blocked in TBS-T 5% milk overnight. Proteins on the membrane will be detected with a primary antibody and if necessary a secondary antibody. Membrane will be washed 3 times between each antibody and prior to development with TBS-T. Membranes will be developed with ECL substrate according to the manufacturer's instructions.

After mRNA co-transfection in mammalian cells in vitro, expression, secretion and coupling of particles to soluble antigens will be detected by WB, by detecting a band of the expected size of the particle and the antigen, similar to what was described in Example 6.

### Example 13—Verification of Nanoparticle Formation when Expressed from Transfected mRNA by Ultracentrifugation and Western Blotting

After Hek cells transfection with mRNA and harvest, the supernatant will be loaded onto an Optiprep step gradient (23, 29 and 35%) followed by centrifuged for 3 h 30 min at 47800 g, 16° C. The gradient will then be dripped into fractions (F1-F12), each fraction containing approximately 250 μL.

If a particle is formed it is expected that it will be found in the middle fractions (F3-F8).

All fractions will be run on denaturing SDS+DTT gels (15 μL loaded). The SDS gel will be transferred on a Nitrocellulose membrane and blocked in TBS-T 5% milk overnight. Proteins on the membrane will be detected with a primary antibody and if necessary a secondary antibody. Membranes will be washed 3 times between each antibody and prior to development with TBS-T. Membranes will be developed with ECL substrate according to the manufacturer's instructions.

After mRNA co-transfection or transfection in mammalian cells in vitro, potential particle formation and secretion will be detected, similar to what was described in Example 7. Particle formation can be expected when the proteins are found within fractions 3-8 from the density gradient.

### Example 14—Visualisation of Particle Formation by Transmission Electronic Microscopy

A described in Example 8, secreted, assembled particles expressed from mRNA constructs in HEK cells will be detected by transmission electronic microscopy.

### Example 15—Verification of Intracellular Conjugation of the Antigen to the Nanoparticle-Forming Protein when Expressed from mRNA

HEK293-Freestyle cells will be transfected or co-transfected with 37.5 μg RNA in 30 mL culture using FreeStyle™ MAX Reagent (16447100, Life Technologies)

After incubation for 6 days, cells and supernatant will harvested. Supernatant will be frozen down at −20° C. and represent the samples called supernatant (SN). Cells will be pelleted by spinning down at 1200 RPM, 5 min, and resuspended in 1×SDS+DTT and frozen down at −20° C. and represent the samples called “cell”.

Both cell and SN will be run on denaturing SDS+DTT gels (15 μL loaded). The SDS gel will be transferred on a Nitrocellulose membrane and blocked in TBS-T 5% milk overnight. Proteins on the membrane will be detected with a primary antibody and if necessary a secondary antibody. Membrane will be washed 3 times between each antibody and prior to development with TBS-T. Membranes will be developed with ECL substrate according to the manufacturer's instructions.

This will allow for visualization of coupling between the particle and the soluble antigen in the cells, similar to what was shown in Example 9. This will show that coupling occurs intracellularly when the constructs are expressed from mRNA.

### Example 16—mRNA Immunization in Mice

mRNA encoding particles and/or soluble antigens (the particle and antigen each fused to a separate peptide tag) will be used for vaccination in mice, similar to what was described in Example 10.

Balb/c mice will be immunized with either a combination of mRNA encoding the particle and mRNA encoding for soluble antigen, or with only mRNA encoding a soluble antigen. Mice will be immunized on day 0 and week 5, and blood will be drawn on week 3 and 4 post prime and post boost.

Serum will be isolated from the blood and run on ELISA for detection of antigen specific IgG. For that purpose, 96-well plates (Nunc MaxiSorp) will be coated overnight at 4° C. with 0.1 μg/well SpyC in PBS. Plates will be blocked for 1 hour at room temperature (RT) using 0.5% skimmed milk in PBS. Mouse serum will be diluted 1:50 in blocking buffer, and added to the plate in a 2-fold dilution, followed by incubation for 1 hour at RT. Plates will be washed three times in PBS in between steps. In order to measure total serum IgG, Horseradish peroxidase (HRP) conjugated goat anti-mouse IgG (Life technologies, A16072) will be diluted 1:1000 in blocking buffer followed by 1 hour incubation at RT. Plates will be developed with TMB X-tra substrate (Kem-En-Tec, 4800A) and absorbance measured at 450 nM. Data will be collected on a BioSan HiPo MPP-96 microplate reader and analyzed using GraphPad Prism (San Diego, USA, version 8.4.3).

It is expected that after mRNA immunization in mice, the mice receiving RNA encoding the particles and soluble antigen (and thus forming coupled antigen-particle complexes) have higher IgG titers against the soluble antigen compared to mice receiving only the RNA encoding for the soluble antigen. Similar to the results shown in Example 10, this trend is expected to be even higher after a boost immunization.

### REFERENCES

De Vincenzo, R.; Conte, C.; Ricci, C.; Scambia, G.; Capelli, G. Long-term efficacy and safety of human papillomavirus vaccination. Int. J. Womens. Health 2014, 6, 999-1010.
Ilva Liekniņa, Gints Kalniņš, Ināra Akopjana, Jānis Bogans, Mihails Šišovs, Juris Jansons, Jānis Rūmnieks, Kaspars Tārs: Production and characterization of novel ssRNA bacteriophage virus-like particles from metagenomic sequencing data; J Nanobiotechnology. 2019; 17: 61.
Schiller, J.; Lowy, D. Explanations for the high potency of HPV prophylactic vaccines. Vaccine 2018, 36, 4768-4773.
Schiller, J. T.; Castellsagué, X.; Garland, S. M. A review of clinical trials of human papillomavirus prophylactic vaccines. Vaccine 2012, 30, F123-F138.
Wadhwa M, Knezevic I, Kang H N, Thorpe R. Immunogenicity assessment of biotherapeutic products: An overview of assays and their utility. Biologicals. 2015; 43(5):298-306. doi:10.1016/j.biologicals.2015.06.004
Young P G, Yosaatmadja Y, Harris P W, Leung I K, Baker E N, Squire C J. Harnessing ester bond chemistry for protein ligation. Chem Commun (Camb). 2017; 53(9):1502-1505. doi:10.1039/c6cc09899a
Zakeri, B. et al. J. Am. Chem. Soc., 2010, 132 (13), pp 4526-4527
Zakeri, B. et al. Proceedings of the National Academy of Sciences 109(12), E690-E697. 2012.
Zhang, P., Narayanan, E., Liu, Q. et al. A multiclade env-gag VLP mRNA vaccine elicits tier-2 HIV-1-neutralizing antibodies and reduces the risk of heterologous SHIV infection in macaques. Nat Med (2021). https://doi.org/10.1038/s41591-021-01574-5

Sequence overview

| | | | |
|---|---|---|---|
| Sequence | | | |
| ID NO: | Organism | Name | Sequence |

Tags (amino acid)

| | | | |
|---|---|---|---|
| 1 | Streptococcus | SpyTag | AHIVMVDAYKPTK |
| | pyogenes | | |
| 2 | Streptococcus | SdyTag | DPIVMIDNDKPIT |
| | dysgalactiae | | |
| 3 | Streptococcus | SnoopTag | KLGDIEFIKVNK |
| | pneumoniae | | |
| 4 | Streptococcus | PhoTag (LPXTG | LVTGTAHIVMVDNYKPIVETGD |
| | phocae | cell wall anchor | |
| | | domain- | |
| | | containing | |
| | | protein) (NCBI | |
| | | Reference | |
| | | Sequence: | |
| | | WP_082385550.1) | |
| 5 | Enterococcus | EntTag | NTIVMVDKLKEVPPT |
| | faecalis | (hypothetical | |
| | | protein | |
| | | CUN42_14770, | |
| | | partial, | |
| | | GenBank | |
| | | PQC83400.1) | |
| 6 | Ruminococcus | RumTag (Cna | SENGNPLIVMVDDTTKVKIS |
| | sp. AF26- | B-type domain- | |
| | 25AA | containing | |
| | | protein) | |
| 7 | Ruminococcus | Rum2Tag | GTPIVIMVDEAKPSLPD |
| | sp. AF25-19 | (hypothetical | |
| | | protein | |
| | | DWY44_14000, | |
| | | partial) | |
| 8 | Ruminococcus | Rum3Tag | GNPLIVMIDEAEQKEIP |
| | sp. Marseille- | (TonB- | |
| | P6503 | dependent | |
| | | receptor) | |
| 9 | Ruminococcus | Rum4Tag | AGGIIVMKDNTTKVSIS |
| | sp. | (hypothetical | |
| | | protein) (NCBI | |
| | | HCW12338.1) | |
| 10 | Ruminococcus | Rum5Tag (Cna | GNPIVTMIDDATLVKIS |
| | flavefaciens | B-type domain- | |
| | | containing | |
| | | protein) (NCBI | |
| | | WP_051536060.1) | |
| 11 | Ruminococcus | Rum6Tag | GNSTITMVDDTTKVHIT |
| | sp. | | |
| 12 | Ruminococcus | Rum7Tag (Cna | GTPLIVMVDDTTKVEIS |
| | sp. AM43-6 | B domain | |
| | | protein) (NCBI | |
| | | WP_118125159.1) | |
| 13 | Artificial | Rumtrunk D9N | GNPLIVMVNDTTKVK |
| | sequence | | |
| 14 | Ruminococcus | Rumtrunk tag | GNPLI VMVDDTTKVK |
| | sp. | | |
| 15 | Bacillus | BacTag (choice- | NEKVTGQFEIVKVDANDKTK |
| | cereus | of-anchor A | |
| | | family protein) | |
| | | (NCBI | |
| | | WP_080470427.1) | |
| 16 | Bacillus | Bac2Tag | SKSLGQFEIVKVDAQDKTK |
| | cereus | (hypothetical | |
| | | protein | |
| | | COD21_31890, | |
| | | partial) (NCBI | |
| | | PGT97799.1) | |
| 17 | Bacillus | Bac3Tag | LGQFEIVKVDSQDKTK |
| | cereus | (choice-of- | |
| | | anchor A family | |
| | | protein) (NCBI | |
| | | WP_053565148.1) | |
| 18 | Bacillus | Bac4Tag | VTGQFEIVKVDAEDKTR |
| | cereus | (choice-of- | |
| | | anchor A family | |
| | | protein) | |
| | | (WP_088344882.1) | |
| 19 | Bacillus | Bac5Tag | EKVMGQFEIMKVDANDKTK |
| | cereus | (hypothetical | |
| | VD022 | protein | |
| | | IC1_02949, | |
| | | partial, | |
| | | EJP89589.1) | |
| 20 | Clostridium | Cpe0147 | DTKQVVKHEDKNDKAQTLVVEKP |
| | perfringens B | (Uniprot: | |
| | str. ATCC | B1R775) aa | |
| | 3626 | 565-587 | |
| | | (esterbond | |
| | | forming TAG) | |

Catchers (amino acid)

| | | | |
|---|---|---|---|
| 21 | Streptococcus | SpyCatcher | GAMVDTLSGLSSEQGQSGDMTIEEDS |
| | pyogenes | | ATHIKESKRDEDGKELAGATMELRDS |
| | | | SGKTISTWISDGQVKDFYLYPGKYTF |
| | | | VETAAPDGYEVATAITFTVNEQGQVT |
| | | | VNGKATKGDAHI |
| 22 | Streptococcus | SdyCatcher | IDTMSGLSGETGQSGNTTIEEDSTTH |
| | dysgalactiae | | VKFSKRDSNGKELAGAMIELRNLSGQ |
| | | | TIQSWVSDGTVKDFYLMPGTYQFVET |
| | | | AAPEGYELAAPITFTIDEKGQIWVDS |
| 23 | Streptococcus | SnoopCatcher | SSGLVPRGSHMKPLRGAVESLQKQHP |
| | pneumoniae | | DYPDIYGAIDQNGTYQNVRTGEDGKL |
| | | | TFKNLSDGKYRLFENSEPAGYKPVON |
| | | | KPIVAFQIVNGEVRDVTSIVPQDIPA |
| | | | TYEFTNGKHYITNEPIPPK |
| 24 | Actinomyces | FimP domain 3 | GSLSKYGKVILTKTGTDDLADKTKYN |
| | viscosus | | GAQFQVYECTKTASGATLRDSDPSTQ |
| | | | TVDPLTIGGEKTFTTAGQGTVEINYL |
| | | | RANDYVNGAKKDQLTDEDYYCLVETK |
| | | | APEGYNLQADPLPERVLAEKAEKKA |
| 25 | Streptococcus | Streptococcal | GSTTKVKLIKVDQDHNRLEGVGFKLV |
| | pneumonia | ancillary pilin | SVARDVSAAAVPLIGEYRYSSSGQVG |
| | serotype 4 | Domain 2 | RTLYTDKNGEIFVTNLPLGNYRFKEV |
| | (strain ATCC | | EPLAGYAVTTLDTDVQLVDHQLVT |
| | BAA- | | |
| | 334/TIGR4) | | |
| 26 | Streptococcus | Streptococcal | PRGNVDFMKVDGRTNTSLQGAMEKVM |
| | pneumonia | ancillary pilin | KEESGHYTPVLQNGKEVVVTSGKDGR |
| | serotype 4 | Domain 3 | FRVEGLEYGTYYLWELQAPTGYVOLT |
| | (strain ATCC | | SPVSFTIGKDTRKELV |
| | BAA- | | |
| | 334/TIGR4) | | |
| 27 | Corynebacterium | Major Pilin | VVTYHGKLKVVKKDGKEAGKVLKGAE |
| | diphtheriae | SpaD Domain 3 | FELYQCTSAAVLGKGPLTVDGVKKWT |
| | NCTC 13129 | | TGDDGTFTIDGLHVTDFEDGKEAAPA |
| | | | TKKFCLKETKAPAGYALPDPNVTEIE |
| | | | FTRAKISEKDKFEGDDEVT |
| 28 | Lactobacillus | Pilin subunit | STNDTTTQNVVLTKYGEDKDVTAIDR |
| | rhamnosus | (SpaA) domain | ATDQIWTGDGAKPLQGVDFTIYNVTA |
| | GG | 2 | NYWASPKDYKGSFDSAPVAATGTIND |
| | | | KGQLTQALPIQSKDASGKTRAAVYLF |
| | | | HETNPRAGYNTSADFWLTLPAKAAAD |
| | | | GNVY |
| 29 | Lactobacillus | Pilin subunit | TTYERTFVKKDAETKEVLEGAGFKIS |
| | rhamnosus | (SpaA) domain | NSDGKFLKLTDKDGQSVSIGEGFIDV |
| | GG | 3 | LANNYRLTWVAESDATVFTSDKSGKF |
| | | | GLNGFADNTTTYTAVETNVPDGYDAA |
| | | | ANTDEKADNS |
| 30 | Streptococcus | Surface protein | GQITIKKIDGSTKASLQGAIFVLKNA |
| | agalactiae | Spb1 domain 3 | TGQFLNENDTNNVEWGTEANATEYTT |
| | A909 | | GADGIITITGLKEGTYYLVEKKAPLG |
| | | | YNLLDNSQKVILGDGATDTTNSDNLL |
| | | | VNP |
| 31 | Streptococcus | PsCsCatcher | EQDVVFSKVNVAGEEIAGAKIQLKDA |
| | intermedius | (LPXTG cell wall | QGQVVHSWTSKAGQSETVKLKAGTYT |
| | | anchor domain- | FHEASAPTGYLAVTDITFEVDVQGKV |
| | | containing | TVKDANGNGVKAD |
| | | protein) | |
| 32 | Streptococcus | RgA Catcher | KLGDIEFIKVNKNDKKPLRGAVESLQ |
| | pneumoniae | | KQHPDYPDIYGAIDQNGTYQNVRTGE |
| | | | DGKLTFKNLSDGKYRLFENSEPAGYK |
| | | | PVQNKPIVAFQIVNGEVRDVTSIVPQ |
| 33 | Corynebacterium | Major Pilin | GSERKGSLTLHKKKGAESEKRATGKE |
| | diphtheriae | SpaD Domain 1 | MDDVAGEPLNGVTFKITKLNEDLONG |
| | NCTC 13129 | | DWAKFPKTAADAKGHETSTTKEVETS |
| | | | GNGTAVFDNLDLGIYLVEETKAPDGI |
| | | | VTGAPFIVSIPMVNEASDAWNYNVVA |
| 34 | Clostridium | Cpe0147 | NLPEVKDGTLRTTVIADGVNGSSEKE |
| | perfringens B | (Uniprot: | ALVSFENSKDGVDVKDTINYEGLVAN |
| | str. ATCC | B1R775) aa | QNYTLTGTLMHVKADGSLEEIATKTT |
| | 3626 | 439-587 | NVTAGENGNGTWGLDFGNQKLQVGEK |
| | | (esterbond | YVVFENAESVENLIDTDKDYNLDTKQ |
| | | forming | VVKHEDKNDKAQTLVVEKP |
| | | CATCHER) | |

Tags (DNA)

| | | | |
|---|---|---|---|
| 35 | Streptococcus | SpyTag DNA | GCTCACATCGTGATGGTGGACGCTTA |
| | pyogenes | | CAAGCCCACCAAG |
| 36 | Streptococcus | SdyTag DNA | GACCCCATCGTGATGATCGACAACGA |
| | dysgalactiae | | CAAGCCCATCACC |
| 37 | Streptococcus | SnoopTag DNA | AAACTGGGTGATATTGAATTTATTAA |
| | pneumoniae | | AGTTAATAAA |
| 38 | Streptococcus | PhoTag DNA | CTGGTTACCGGCACCGCACATATTGT |
| | phocae | | TATGGTTGATAACTATAAGCCGATCG |
| | | | TGGAAACCGGTGAT |
| 39 | Enterococcus | EntTag DNA | ACCGAAGTTAGCGGTAATACCATTGT |
| | faecalis | | GATGGTGGATAAACTGAAAGAAGTTC |
| | | | CGCCTACC |
| 40 | Ruminococcus | RumTag DNA | AGCGAAAACGGCAACCCGCTGATTGT |
| | sp. AF26- | | GATGGTGGATGATACCACCAAAGTGA |
| | 25AA | | AAATTAGC |
| 41 | Ruminococcus | Rum2Tag DNA | GGCACCCCGATTGTGATTATGGTGGA |
| | sp. AF25-19 | | TGAAGCGAAACCGAGCCTGCCGGAT |
| 42 | Ruminococcus | Rum3Tag DNA | GGCAACCCGCTGATTGTGATGATTGA |
| | sp. Marseille- | | TGAAGCGGAACAGAAAGAAATTCCG |
| | P6503 | | |
| 43 | Ruminococcus | Rum4Tag DNA | GCGGGCGGCATTATTGTGATGAAAGA |
| | sp. | | TAACACCACCAAAGTGAGCATTAGC |
| 44 | Ruminococcus | Rum5Tag DNA | GGCAACCCGATTGTGACCATGATTGA |
| | flavefaciens | | TGATGCGACCCTGGTGAAAATT |
| 45 | Ruminococcus | Rum6Tag DNA | GGCAACAGCACCATTACCATGGTGGA |
| | sp. | | TGATACCACCAAAGTGCATATTACC |
| 46 | Ruminococcus | Rum7Tag DNA | GGCACCCCGCTGATTGTGATGGTGGA |
| | sp. AM43-6 | | TGATACCACCAAAGTGGAAATTAGC |
| 47 | Artificial | Rumtrunk D9N | GGTAATCCGCTGATTGTGATGGTGAA |
| | sequence | DNA | TGATACCACCAAAGTGAAA |
| 48 | Ruminococcus | Rumtrunk tag | GGCAACCCGCTGATTGTGATGGTGGA |
| | sp. | | TGATACCACCAAAGTGAAA |
| 49 | Bacillus | BacTag DNA | GGTCAGTTCGAAATTGTTAAAGTTGA |
| | cereus | | TGCAAACGATAAAACTAAA |
| 50 | Bacillus | Bac2Tag DNA | AGCAAAAGCCTGGGCCAGTTTGAAAT |
| | cereus | | TGTGAAAGTGGATGCGCAGGATAAAA |
| | | | CCAAA |
| 51 | Bacillus | Bac3Tag DNA | CTGGGCCAGTTTGAAATTGTTAAAGT |
| | cereus | | TGATAGCCAGGATAAAACCAAA |
| 52 | Bacillus | Bac4Tag DNA | GTTACCGGTCAGTTTGAAATCGTTAA |
| | cereus | | AGTTGATGCCGAAGATAAGACCCGT |
| 53 | Bacillus | Bac5Tag DNA | GAAAAAGTGATGGGCCAGTTCGAAAT |
| | cereus | | CATGAAAGTTGATGCCAACGACAAGA |
| | VD022 | | CCAAA |
| 54 | Clostridium | cpe0147 | GACACCAAGCAGGTGGTGAAGCACGA |
| | perfringens B | (Uniprot: | GGACAAGAACGACAAGGCCCAGACCC |
| | str. ATCC | B1R775) aa | TGGTGGTGGAGAAGCCC |
| | 3626 | 565-587 | |
| | | (esterbond | |
| | | forming TAG) | |
| | | DNA | |
| | | Catchers (DNA) | |
| 55 | Streptococcus | SpyCatcher | GGTGCAATGGTTGATACCCTGAGCGG |
| | pyogenes | DNA | TCTGAGCAGCGAACAGGGTCAGAGCG |
| | | | GTGATATGACCATTGAAGAAGATAGC |
| | | | GCAACCCACATCAAATTCAGCAAACG |
| | | | TGATGAAGATGGTAAAGAACTGGCAG |
| | | | GCGCAACAATGGAACTGCGTGATAGC |
| | | | AGCGGTAAAACCATTAGCACCTGGAT |
| | | | TAGTGATGGTCAGGTGAAAGATTTTT |
| | | | ATCTGTACCCTGGCAAATACACCTTT |
| | | | GTTGAAACCGCAGCACCGGATGGTTA |
| | | | TGAAGTTGCAACCGCAATTACCTTTA |
| | | | CCGTTAATGAACAGGGCCAGGTTACC |
| | | | GTGAATGGTAAAGCAACCAAAGGTGA |
| | | | TGCACATATT |
| 56 | Streptococcus | SdyCatcher | ATGGGTATTGATACCATGAGCGGTCT |
| | dysgalactiae | DNA | GAGCGGTGAAACCGGTCAGAGCGGTA |
| | | | ATACCACCATTGAAGAGGATAGCACC |
| | | | ACACATGTGAAATTCAGCAAACGCGA |
| | | | TGCAAACGGCAAAGAACTGGCAGGCG |
| | | | CAATGATTGAACTGCGTAATCTGAGT |
| | | | GGTCAGACCATTCAGAGCTGGGTTAG |
| | | | TGATGGCACCGTTAAAGATTTTTATC |
| | | | TGATGCCTGGCACCTATCAGTTTGTT |
| | | | GAAACCGCAGCACCGGAAGGTTATGA |
| | | | GCTGGCAGCACCGATTACCTTTACCA |
| | | | TTGATGAAAAAGGTCAGATTTGGGTT |
| | | | GATAGC |
| 57 | Streptococcus | SnoopCatcher | AGCAGCGGCCTGGTGCCGCGCGGCAG |
| | pneumoniae | DNA | CCATATGAAGCCGCTGCGTGGTGCCG |
| | | | TGTTTAGCCTGCAGAAACAGCATCCC |
| | | | GACTATCCCGATATCTATGGCGCGAT |
| | | | TGATCAGAATGGGACCTATCAAAATG |
| | | | TGCGTACCGGCGAAGATGGTAAACTG |
| | | | ACCTTTAAGAATCTGAGCGATGGCAA |
| | | | ATATCGCCTGTTTGAAAATAGCGAAC |
| | | | CCGCTGGCTATAAACCGGTGCAGAAT |
| | | | AAGCCGATTGTGGCGTTTCAGATTGT |
| | | | GAATGGCGAAGTGCGTGATGTGACCA |
| | | | GCATTGTGCCGCAGGATATTCCGGCT |
| | | | ACATATGAATTTACCAACGGTAAACA |
| | | | TTATATCACCAATGAACCGATACCGC |
| | | | CGAAA |
| 58 | Actinomyces | FimP domain 3 | GGTAGCCTGAGCAAATATGGTAAAGT |
| | viscosus | DNA | GATTCTGACCAAAACCGGCACCGATG |
| | | | ATCTGGCAGATAAAACCAAATATAAC |
| | | | GGTGCACAGTTTCAGGTGTATGAATG |
| | | | TACCAAAACAGCAAGCGGTGCAACCC |
| | | | TGCGTGATAGCGATCCGAGCACACAG |
| | | | ACCGTTGATCCGCTGACCATTGGTGG |
| | | | TGAAAAAACCTTTACCACCGCAGGTC |
| | | | AGGGCACCGTTGAAATTAATTATCTG |
| | | | CGTGCCAATGATTATGTGAACGGTGC |
| | | | AAAAAAAGATCAGCTGACCGATGAAG |
| | | | ATTATTACTGTCTGGTTGAAACCAAA |
| | | | GCACCGGAAGGTTATAATCTGCAGGC |
| | | | AGATCCGCTGCCGTTTCGTGTTCTGG |
| | | | CCGAAAAAGCAGAAAAAAAAGCC |
| 59 | Streptococcus | Streptococcal | GGTAGCACCACCAAAGTGAAACTGAT |
| | pneumonia | ancillary pilin | TAAAGTTGATCAGGATCACAATCGTC |
| | serotype 4 | Domain 2 DNA | TGGAAGGTGTTGGTTTTAAACTGGTT |
| | (strain ATCC | | AGCGTTGCACGTGATGTTAGCGCAGC |
| | BAA- | | AGCAGTTCCGCTGATTGGTGAATATC |
| | 334/TIGR4) | | GTTATAGCAGCAGCGGTCAGGTTGGT |
| | | | CGTACCCTGTATACCGATAAAAATGG |
| | | | CGAAATTTTCGTTACCAATCTGCCGC |
| | | | TGGGTAACTATCGTTTTAAAGAAGTT |
| | | | GAACCGCTGGCAGGTTATGCAGTTAC |
| | | | CACACTGGATACCGATGTTCAGCTGG |
| | | | TTGATCATCAGCTGGTGACC |
| 60 | Streptococcus | Streptococcal | CCGCGTGGTAATGTTGATTTTATGAA |
| | pneumonia | ancillary pilin | AGTTGATGGTCGCACCAATACCAGCC |
| | serotype 4 | Domain 3 DNA | TGCAGGGTGCAATGTTTAAAGTGATG |
| | (strain ATCC | | AAAGAAGAAAGCGGTCACTATACACC |
| | BAA- | | GGTGCTGCAGAATGGTAAAGAAGTTG |
| | 334/TIGR4) | | TTGTTACCAGCGGTAAAGATGGTCGT |
| | | | TTTCGTGTTGAAGGTCTGGAATATGG |
| | | | CACCTATTATCTGTGGGAACTGCAGG |
| | | | CACCGACCGGTTATGTTCAGCTGACC |
| | | | AGTCCGGTTAGTTTTACCATTGGCAA |
| | | | AGATACCCGTAAAGAACTGGTG |
| 61 | Corynebacterium | Major Pilin | GTTGTTACCTATCATGGTAAACTGAA |
| | diphtheriae | SpaD Domain 3 | AGTGGTGAAAAAAGACGGTAAAGAGG |
| | NCTC 13129 | DNA | CAGGCAAAGTTCTGAAAGGTGCAGAA |
| | | | TTTGAACTGTATCAGTGTACCAGCGC |
| | | | AGCAGTTTTAGGTAAAGGTCCGCTGA |
| | | | CCGTTGATGGTGTGAAAAAATGGACC |
| | | | ACCGGTGATGATGGCACCTTTACCAT |
| | | | TGATGGTCTGCATGTTACCGATTTTG |
| | | | AAGATGGTAAAGAAGCCGCACCGGCA |
| | | | ACCAAAAAATTCTGTCTGAAAGAAAC |
| | | | CAAAGCACCGGCAGGTTATGCACTGC |
| | | | CTGATCCGAATGTGACCGAAATTGAA |
| | | | TTTACCCGTGCAAAAATCAGCGAGAA |
| | | | AGATAAATTTGAAGGCGACGATGAAG |
| | | | TGACC |
| 62 | Lactobacillus | Pilin subunit | AGCACCAATGATACCACCACACAGAA |
| | rhamnosus | (SpaA) domain | TGTTGTTCTGACCAAATATGGCTTCG |
| | GG | 2 DNA | ATAAAGATGTTACCGCAATTGATCGT |
| | | | GCAACCGATCAGATTTGGACCGGTGA |
| | | | TGGTGCAAAACCGCTGCAGGGTGTTG |
| | | | ATTTTACCATTTATAACGTGACCGCC |
| | | | AATTATTGGGCAAGCCCGAAAGATTA |
| | | | TAAAGGCAGCTTTGATAGCGCACCGG |
| | | | TTGCAGCCACCGGTACAACAAATGAT |
| | | | AAAGGCCAGCTGACCCAGGCACTGCC |
| | | | GATTCAGAGCAAAGATGCAAGCGGTA |
| | | | AAACCCGTGCAGCAGTTTACCTGTTT |
| | | | CACGAAACCAATCCGCGTGCAGGTTA |
| | | | TAATACCAGCGCAGATTTTTGGCTGA |
| | | | CCCTGCCTGCAAAAGCAGCAGCAGAT |
| | | | GGTAATGTTTAT |
| 63 | Lactobacillus | Pilin subunit | ACCACCTATGAACGTACCTTTGTTAA |
| | rhamnosus | (SpaA) domain | AAAAGACGCCGAAACCAAAGAAGTTC |
| | GG | 3 DNA | TGGAAGGCGCAGGCTTTAAAATCAGC |
| | | | AATAGTGATGGCAAATTCCTGAAACT |
| | | | GACCGATAAAGATGGTCAGAGCGTTA |
| | | | GCATTGGTGAAGGTTTTATTGATGTT |
| | | | CTGGCCAATAACTATCGTCTGACCTG |
| | | | GGTTGCAGAAAGTGATGCAACCGTTT |
| | | | TTACCAGCGATAAAAGCGGCAAATTT |
| | | | GGTCTGAATGGTTTTGCAGATAATAC |
| | | | CACCACCTATACCGCAGTTGAAACCA |
| | | | ATGTTCCGGATGGTTATGATGCAGCA |
| | | | GCAAACACCGATTTCAAAGCCGATAA |
| | | | TAGC |
| 64 | Streptococcus | Surface protein | GGTCAGATTACCATCAAAAAAATCGA |
| | agalactiae | Spb1 domain 3 | TGGTAGCACCAAAGCAAGCCTGCAGG |
| | A909 | DNA | GTGCAATTTTTGTTCTGAAAAATGCA |
| | | | ACCGGTCAGTTCCTGAATTTTAACGA |
| | | | TACCAATAATGTTGAATGGGGCACCG |
| | | | AAGCAAATGCCACCGAATATACCACC |
| | | | GGTGCAGATGGTATTATTACCATTAC |
| | | | CGGTCTGAAAGAAGGCACCTATTACC |
| | | | TGGTTGAAAAAAAAGCACCGCTGGGT |
| | | | TATAATCTGCTGGATAATTCACAGAA |
| | | | AGTGATTTTAGGTGATGGTGCAACCG |
| | | | ATACCACCAATAGCGATAACCTGCTG |
| | | | GTTAATCCG |
| 65 | Streptococcus | PsCsCatcher | GAACAGGATGTTGTGTTTAGCAAAGT |
| | intermedius | (LPXTG cell wall | TAATGTTGCCGGTGAAGAAATTGCGG |
| | | anchor domain- | GTGCAAAAATCCAGCTGAAAGATGCA |
| | | containing | CAGGGTCAAGTTGTTCATAGCTGGAC |
| | | protein) DNA | CAGCAAAGCAGGTCAGAGCGAAACCG |
| | | | TTAAACTGAAAGCAGGCACCTATACC |
| | | | TTTCATGAAGCAAGCGCACCGACCGG |
| | | | TTATCTGGCAGTTACCGATATTACCT |
| | | | TTGAAGTTGATGTTCAGGGTAAAGTG |
| | | | ACCGTTAAAGATGCAAATGGTAATGG |
| | | | TGTGAAAGCCGAC |
| 66 | Streptococcus | RgA Catcher | AAACTGGGTGATATTGAGTTCATCAA |
| | pneumoniae | DNA | AGTGAACAAAAACGATAAAAAACCGC |
| | | | TGCGTGGTGCAGTTTTTAGCCTGCAG |
| | | | AAACAGCATCCGGATTACCCGGATAT |
| | | | TTATGGTGCAATTGATCAGAATGGCA |
| | | | CCTATCAGAATGTTCGTACCGGTGAA |
| | | | GATGGTAAACTGACCTTTAAAAACCT |
| | | | GAGCGACGGTAAATATCGCCTGTTTG |
| | | | AAAATAGCGAACCGGCAGGTTATAAA |
| | | | CCGGTTCAGAATAAACCGATTGTGGC |
| | | | CTTTCAGATTGTTAATGGTGAAGTTC |
| | | | GTGATGTGACCAGCATTGTTCCGCAG |
| 67 | Corynebacterium | Major Pilin | GGTAGCGAACGTAAAGGTAGTCTGAC |
| | diphtheriae | SpaD Domain 1 | CCTGCATAAAAAGAAAGGTGCAGAAA |
| | NCTC 13129 | DNA | GCGAAAAACGTGCAACCGGTAAAGAA |
| | | | ATGGATGATGTTGCCGGTGAACCGCT |
| | | | GAATGGTGTTACCTTTAAAATCACCA |
| | | | AACTGAACTTCGATCTGCAGAATGGT |
| | | | GATTGGGCAAAATTTCCGAAAACCGC |
| | | | AGCAGATGCAAAAGGTCATGAAACCA |
| | | | GCACCACCAAAGAAGTGGAAACCAGC |
| | | | GGTAATGGCACCGCAGTTTTTGATAA |
| | | | TCTGGATCTGGGTATTTACCTGGTGG |
| | | | AAGAAACCAAAGCACCGGATGGTATT |
| | | | GTTACAGGTGCACCGTTTATTGTTAG |
| | | | CATTCCGATGGTTAATGAAGCAAGTG |
| | | | ATGCCTGGAATTATAACGTTGTTGCA |
| 68 | Clostridium | cpe0147(uniprot: | AACCTGCCCGAGGTGAAGGACGGCAC |
| | perfringens B | B1R775) aa | CCTGAGGACCACCGTGATCGCCGACG |
| | str. ATCC | 439-587 | GCGTGAACGGCAGCAGCGAGAAGGAG |
| | 3626 | (esterbond | GCCCTGGTGAGCTTCGAGAACAGCAA |
| | | forming catcher) | GGACGGCGTGGACGTGAAGGACACCA |
| | | DNA | TCAACTACGAGGGCCTGGTGGCCAAC |
| | | | CAGAACTACACCCTGACCGGCACCCT |
| | | | GATGCACGTGAAGGCCGACGGCAGCC |
| | | | TGGAGGAGATCGCCACCAAGACCACC |
| | | | AACGTGACCGCCGGCGAGAACGGCAA |
| | | | CGGCACCTGGGGCCTGGACTTCGGCA |
| | | | ACCAGAAGCTGCAGGTGGGCGAGAAG |
| | | | TACGTGGTGTTCGAGAACGCCGAGAG |
| | | | CGTGGAGAACCTGATCGACACCGACA |
| | | | AGGACTACAACCTGGACACCAAGCAG |
| | | | GTGGTGAAGCACGAGGACAAGAACGA |
| | | | CAAGGCCCAGACCCTGGTGGTGGAGA |
| | | | AGCCC |

Particle-forming proteins (amino acid)

| | | | |
|---|---|---|---|
| 69 | Homo sapiens | Ferritin, aa | RMLKALNDQLNRELYSAYLYFAMAAY |
| | | 5-174 | FEDLGLEGFANWMKAQAEEEIGHALR |
| | | | FYNYIYDRNGRVELDEIPKPPKEWES |
| | | | PLKAFEAAYEHEKFISKSIYELAALA |
| | | | EEEKDYSTRAFLEWFINEQVEEEASV |
| | | | KKILDKLKFAKDSPQILFMLDKELSA |
| | | | RAPKLPGLLMQGGE |
| 70 | Homo sapiens | Lumazine | MQIYEGKLTAEGLRFGIVASRENHAL |
| | | synthase | VDRLVEGAIDCIVRHGGREEDITLVR |
| | | | VPGSWEIPVAAGELARKEDIDAVIAI |
| | | | GVLIRGATPHEDYIASEVSKGLANLS |
| | | | LELRKPITFGVITADTLEQAIERAGT |
| | | | KHGNKGWEAALSAIEMANLFKSLR |
| 71 | Homo sapiens | NSP10 | PANSTVLSFCAFAVDPAKAYKDYLAS |
| | | | GGQPITNCVKMLCTHTGTGQAITVTP |
| | | | EANMDQESFGGASCCLYCRCHIDHPN |
| | | | PKGFCDLKGKYVQIPTTCANDPVGFT |
| | | | LRNTVCTVCGMWKGYGCS |
| 72 | Homo sapiens | 1301 | MKMEELFKKHKIVAVLRANSVEEAKK |
| | | | KALAVELGGVHLIEITFTVPDADTVI |
| | | | KELSFLKEMGAIIGAGTVTSVEQARK |
| | | | AVESGAEFIVSPHLDEEISQFAKEKG |
| | | | VFYMPGVMTPTELVKAMKLGHTILKL |
| | | | FPGEVVGPQFVKAMKGPFPNVKFVPT |
| | | | GGVNLDNVCEWEKAGVLAVGVGSALV |
| | | | KGTPVEVAEKAKAFVEKIRGCTE |
| 73 | Thermotoga | 5KP9 | MGARASGSKSGSGSDSGSKMEELFKK |
| | maritima | | HKIVAVLRANSVEEAKKKALAVELGG |
| | | | VHLIEITFTVPDADTVIKELSELKEM |
| | | | GAIIGAGTVTSVEQCRKAVESGAEFI |
| | | | VSPHLDEEISQFCKEKGVFYMPGVMT |
| | | | PTELVKAMKLGHTILKLFPGEVVGPQ |
| | | | FVKAMKGPFPNVKFVPTGGVNLDNVC |
| | | | EWFKAGVLAVGVGSALVKGTPVEVAE |
| | | | KAKAFVEKIRGCTEQKLISEEDLQSR |
| | | | PEPTAPPEESERSGVETTTPPQKQEP |
| | | | IDKELYPLTSLRSLEGNDPSSQ |

Particle-forming proteins (DNA)

| | | | |
|---|---|---|---|
| 74 | Homo sapiens | Ferritin DNA | AGAATGCTGAAGGCCCTGAACGACCA |
| | | sequence | GCTGAACAGAGAGCTGTACTCCGCCT |
| | | covering aa | ACCTGTACTTCGCTATGGCCGCCTAC |
| | | 5-174 | TTCGAGGACCTGGGCCTTGAGGGATT |
| | | | CGCCAACTGGATGAAGGCTCAGGCCG |
| | | | AGGAAGAGATCGGCCACGCTCTGAGA |
| | | | TTCTACAACTACATCTACGACAGAAA |
| | | | CGGCCGCGTGGAACTGGACGAGATCC |
| | | | CCAAGCCTCCTAAAGAGTGGGAGAGC |
| | | | CCTCTGAAGGCTTTCGAGGCTGCTTA |
| | | | CGAGCACGAGAAGTTCATCAGCAAGA |
| | | | GCATCTACGAGCTGGCCGCTCTGGCC |
| | | | GAAGAGGAAAAGGACTACTCTACCAG |
| | | | AGCCTTCCTGGAATGGTTCATCAACG |
| | | | AACAGGTGGAAGAGGAAGCCAGCGTC |
| | | | AAGAAGATCCTGGACAAGCTGAAGTT |
| | | | CGCCAAGGACAGCCCTCAGATCCTGT |
| | | | TCATGCTGGACAAAGAGCTGAGCGCC |
| | | | AGGGCTCCTAAACTGCCTGGACTGCT |
| | | | TATGCAAGGCGGCGAA |
| 75 | Homo sapiens | Lumazine | ATGCAGATCTACGAGGGCAAGCTGAC |
| | | synthase DNA | CGCCGAGGGCCTGAGGTTCGGCATCG |
| | | | TGGCCAGCAGGTTCAACCACGCCCTG |
| | | | GTGGACAGGCTGGTGGAGGGCGCCAT |
| | | | CGACTGCATCGTGAGGCACGGCGGCA |
| | | | GGGAGGAGGACATCACCCTGGTGAGG |
| | | | GTGCCCGGCAGCTGGGAGATCCCCGT |
| | | | GGCCGCCGGCGAGCTGGCCAGGAAGG |
| | | | AGGACATCGACGCCGTGATCGCCATC |
| | | | GGCGTGCTGATCAGGGGCGCCACCCC |
| | | | CCACTTCGACTACATCGCCAGCGAGG |
| | | | TGAGCAAGGGCCTGGCCAACCTGAGC |
| | | | CTGGAGCTGAGGAAGCCCATCACCTT |
| | | | CGGCGTGATCACCGCCGACACCCTGG |
| | | | AGCAGGCCATCGAGAGGGCCGGCACC |
| | | | AAGCACGGCAACAAGGGCTGGGAGGC |
| | | | CGCCCTGAGCGCCATCGAGATGGCCA |
| | | | ACCTGTTCAAGAGCCTGAGG |
| 76 | Homo sapiens | NSP10 DNA | CCCGCCAACAGCACCGTGCTGAGCTT |
| | | | CTGCGCCTTCGCCGTGGACCCCGCCA |
| | | | AGGCCTACAAGGACTACCTGGCCAGC |
| | | | GGCGGCCAGCCCATCACCAACTGCGT |
| | | | GAAGATGCTGTGCACCCACACCGGCA |
| | | | CCGGCCAGGCCATCACCGTGACCCCC |
| | | | GAGGCCAACATGGACCAGGAGAGCTT |
| | | | CGGCGGCGCCAGCTGCTGCCTGTACT |
| | | | GCAGGTGCCACATCGACCACCCCAAC |
| | | | CCCAAGGGCTTCTGCGACCTGAAGGG |
| | | | CAAGTACGTGCAGATCCCCACCACCT |
| | | | GCGCCAACGACCCCGTGGGCTTCACC |
| | | | CTGAGGAACACCGTGTGCACCGTGTG |
| | | | CGGCATGTGGAAGGGCTACGGCTGCA |
| | | | GC |
| 77 | Homo sapiens | 1301 DNA | ATGAAGATGGAGGAGCTGTTCAAGAA |
| | | sequence | GCACAAGATCGTGGCCGTGCTGAGGG |
| | | (optimized for | CCAACAGCGTGGAGGAGGCCAAGAAG |
| | | human | AAGGCCCTGGCCGTGTTCCTGGGCGG |
| | | expression) | CGTGCACCTGATCGAGATCACCTTCA |
| | | | CCGTGCCCGACGCCGACACCGTGATC |
| | | | AAGGAGCTGAGCTTCCTGAAGGAGAT |
| | | | GGGCGCCATCATCGGCGCCGGCACCG |
| | | | TGACCAGCGTGGAGCAGGCCAGGAAG |
| | | | GCCGTGGAGAGCGGCGCCGAGTTCAT |
| | | | CGTGAGCCCCCACCTGGACGAGGAGA |
| | | | TCAGCCAGTTCGCCAAGGAGAAGGGC |
| | | | GTGTTCTACATGCCCGGCGTGATGAC |
| | | | CCCCACCGAGCTGGTGAAGGCCATGA |
| | | | AGCTGGGCCACACCATCCTGAAGCTG |
| | | | TTCCCCGGCGAGGTGGTGGGCCCCCA |
| | | | GTTCGTGAAGGCCATGAAGGGCCCCT |
| | | | TCCCCAACGTGAAGTTCGTGCCCACC |
| | | | GGCGGCGTGAACCTGGACAACGTGTG |
| | | | CGAGTGGTTCAAGGCCGGCGTGCTGG |
| | | | CCGTGGGCGTGGGCAGCGCCCTGGTG |
| | | | AAGGGCACCCCCGTGGAGGTGGCCGA |
| | | | GAAGGCCAAGGCCTTCGTGGAGAAGA |
| | | | TCAGGGGCTGCACCGAG |

Signal peptides (amino acid)

| | | | |
|---|---|---|---|
| 78 | Thermotoga | 5KP9 DNA | ATGGGCGCCAGGGCCAGCGGCAGCAA |
| | maritima | | GAGCGGCAGCGGCAGCGACAGCGGCA |
| | | | GCAAGATGGAGGAGCTGTTCAAGAAG |
| | | | CACAAGATCGTGGCCGTGCTGAGGGC |
| | | | CAACAGCGTGGAGGAGGCCAAGAAGA |
| | | | AGGCCCTGGCCGTGTTCCTGGGCGGC |
| | | | GTGCACCTGATCGAGATCACCTTCAC |
| | | | CGTGCCCGACGCCGACACCGTGATCA |
| | | | AGGAGCTGAGCTTCCTGAAGGAGATG |
| | | | GGCGCCATCATCGGCGCCGGCACCGT |
| | | | GACCAGCGTGGAGCAGTGCAGGAAGG |
| | | | CCGTGGAGAGCGGCGCCGAGTTCATC |
| | | | GTGAGCCCCCACCTGGACGAGGAGAT |
| | | | CAGCCAGTTCTGCAAGGAGAAGGGCG |
| | | | TGTTCTACATGCCCGGCGTGATGACC |
| | | | CCCACCGAGCTGGTGAAGGCCATGAA |
| | | | GCTGGGCCACACCATCCTGAAGCTGT |
| | | | TCCCCGGCGAGGTGGTGGGCCCCCAG |
| | | | TTCGTGAAGGCCATGAAGGGCCCCTT |
| | | | CCCCAACGTGAAGTTCGTGCCCACCG |
| | | | GCGGCGTGAACCTGGACAACGTGTGC |
| | | | GAGTGGTTCAAGGCCGGCGTGCTGGC |
| | | | CGTGGGCGTGGGCAGCGCCCTGGTGA |
| | | | AGGGCACCCCCGTGGAGGTGGCCGAG |
| | | | AAGGCCAAGGCCTTCGTGGAGAAGAT |
| | | | CAGGGGCTGCACCGAGCAGAAGCTGA |
| | | | TCAGCGAGGAGGACCTGCAGAGCAGG |
| | | | CCCGAGCCCACCGCCCCCCCCGAGGA |
| | | | GAGCTTCAGGAGCGGCGTGGAGACCA |
| | | | CCACCCCCCCCCAGAAGCAGGAGCCC |
| | | | ATCGACAAGGAGCTGTACCCCCTGAC |
| | | | CAGCCTGAGGAGCCTGTTCGGCAACG |
| | | | ACCCCAGCAGCCAG |

Signal peptides (amino acid)

| | | | |
|---|---|---|---|
| 79 | Homo sapiens | SP of azurocidin | MTRLTVLALLAGLLASSRA |
| 80 | Homo sapiens | Serum albumin | MKWVTFISLLELESSAYS |
| 81 | Homo sapiens | Modified serum | MKWVTFISLLELFSSSSRA |
| | | albumin | |
| 82 | Cricetulus | Ig kappa chain | MGSAALLLWVLLLWVPGSNG |
| | griseus | V III region | |
| 83 | Cricetulus | Modified Ig | MGSAALLLWVLLLWVPSSRA |
| | griseus | kappa chain V | |
| | | Ill region MOPC | |
| | | 63 like (mlgκ C) | |
| 84 | Homo sapiens | Modified Ig | MEAPAQLLFLLLLWLPSSRA |
| | | kappa chain V | |
| | | III region VG | |
| | | (mlgκ H) | |

Others

| | | | |
|---|---|---|---|
| 85 | Artificial | pVax1 | AHIVMVDAYKPTKGSGTAGGGSGSAN |
| | | SpyTag-AP205 | KPMQPITSTANKIVWSDPTRLSTTES |
| | | (amino acid) | ASLLRQRVKVGIAELNNVSGQYVSVY |
| | | | KRPAPKPEGCADACVIMPNENQSIRT |
| | | | VISGSAENLATLKAEWETHKRNVDTL |
| | | | FASGNAGLGELDPTAAIVSSDTTA |
| 86 | Artificial | pVAX1_HBc- | MDIDPYKEFGASVELLSFLPSDFFPS |
| | | SpyTag (amino | IRDLLDTASALYREALESPEHVSPHH |
| | | acid) | TALRQAILCWGELMNLATWVGSNLED |
| | | | PASRELVVSYVNVNMGLKLRQILWFH |
| | | | ISCLTFGRETVLEYLVSFGVWIRTPT |
| | | | AYRPPNAPILSTLPETTVVGGGGGSP |
| | | | GGGTPSPGGGGSQSPGGGGSQSGESQ |
| | | | CGSAHIVMVDAYKPTK |
| 87 | Artificial | pVAX1- | GAMVDTLSGLSSEQGQSGDMTIEEDS |
| | | SPYCEGFP | ATHIKESKRDEDGKELAGATMELRDS |
| | | (amino acid) | SGKTISTWISDGQVKDFYLYPGKYTF |
| | | | VETAAPDGYEVATAITFTVNEQGQVT |
| | | | VNGKATKGDAHIGGSGSMVSKGEELF |
| | | | TGVVPILVELDGDVNGHKFSVSGEGE |
| | | | GDATYGKLTLKFICTTGKLPVPWPTL |
| | | | VTTLTYGVQCFSRYPDHMKQHDFFKS |
| | | | AMPEGYVQERTIFFKDDGNYKTRAEV |
| | | | KFEGDTLVNRIELKGIDFKEDGNILG |
| | | | HKLEYNYNSHNVYIMADKQKNGIKVN |
| | | | FKIRHNIEDGSVQLADHYQQNTPIGD |
| | | | GPVLLPDNHYLSTQSALSKDPNEKRD |
| | | | HMVLLEFVTAAGITLGMDELYK |
| 88 | Artificial | pVax1 | GCTCACATCGTGATGGTGGACGCCTA |
| | | SpyTag-AP205 | CAAGCCCACAAAAGGCTCTGGAACAG |
| | | (DNA) | CTGGCGGCGGATCTGGCTCTGCCAAC |
| | | | AAACCTATGCAGCCCATCACCAGCAC |
| | | | CGCCAACAAGATCGTTTGGAGCGACC |
| | | | CCACCAGACTGAGCACCACATTCAGC |
| | | | GCTAGCCTGCTGAGACAGCGCGTGAA |
| | | | AGTGGGAATCGCCGAGCTGAACAATG |
| | | | TGTCCGGCCAGTACGTGTCCGTGTAC |
| | | | AAGAGGCCTGCTCCTAAGCCTGAGGG |
| | | | CTGTGCCGATGCCTGTGTGATCATGC |
| | | | CCAACGAGAACCAGAGCATCAGAACC |
| | | | GTGATCAGCGGCAGCGCCGAGAACCT |
| | | | GGCTACACTGAAGGCTGAGTGGGAGA |
| | | | CACACAAGAGAAACGTGGACACCCTG |
| | | | TTCGCCTCTGGCAACGCTGGACTGGG |
| | | | CTTCCTGGATCCTACAGCCGCTATCG |
| | | | TGTCTAGCGACACCACAGCCTGA |
| 89 | Artificial | pVAX1_HBc- | ATGGACATCGACCCCTACAAAGAATT |
| | | SpyTag (DNA) | TGGCGCCAGCGTGGAACTGCTGAGCT |
| | | | TCCTGCCTAGCGACTTCTTCCCTTCC |
| | | | ATCCGGGACCTGCTGGATACAGCCAG |
| | | | CGCACTGTATAGAGAGGCCCTGGAAT |
| | | | CTCCCGAGCACGTGTCACCTCACCAC |
| | | | ACAGCTCTGAGACAGGCCATCCTGTG |
| | | | TTGGGGCGAGCTGATGAACCTGGCCA |
| | | | CATGGGTCGGAAGCAACCTGGAAGAT |
| | | | CCCGCCAGCAGAGAACTGGTGGTGTC |
| | | | CTACGTGAACGTGAACATGGGCCTGA |
| | | | AGCTGAGACAGATCCTGTGGTTCCAC |
| | | | ATCAGCTGCCTGACCTTCGGCAGAGA |
| | | | AACCGTGCTGGAATACCTGGTGTCCT |
| | | | TCGGCGTGTGGATCAGAACCCCTACC |
| | | | GCCTACAGACCTCCTAACGCTCCCAT |
| | | | CCTGAGCACCCTGCCTGAGACAACAG |
| | | | TTGTTGGCGGAGGCGGAGGATCTCCT |
| | | | GGCGGAGGAACACCTTCTCCAGGTGG |
| | | | TGGTGGATCTCAAAGTCCTGGTGGTG |
| | | | GCGGTTCTCAGAGCGGCGAATCTCAG |
| | | | TGTGGCTCTGCCCACATCGTGATGGT |
| | | | GGACGCCTACAAGCCCACCAAATGA |
| 90 | Artificial | pVAX1- | GAGCCATGGTGGATACACTGTCTGGA |
| | | SPYCEGFP | CTGTCTAGCGAGCAGGGCCAGAGCGG |
| | | (DNA) | CGACATGACAATCGAGGAAGATAGCG |
| | | | CCACACACATCAAGTTCAGCAAGCGC |
| | | | GACGAGGACGGCAAAGAACTGGCTGG |
| | | | CGCTACCATGGAACTGAGAGACAGCA |
| | | | GCGGCAAGACCATCAGCACCTGGATC |
| | | | TCTGACGGCCAAGTGAAGGACTTCTA |
| | | | TCTGTACCCCGGCAAGTACACCTTCG |
| | | | TGGAAACAGCCGCTCCTGACGGATAC |
| | | | GAGGTGGCCACAGCCATCACCTTCAC |
| | | | CGTGAACGAGCAGGGACAAGTGACAG |
| | | | TGAACGGCAAGGCCACAAAGGGCGAC |
| | | | GCTCACATTGGCGGCTCTGGCTCCAT |
| | | | GGTGTCCAAGGGCGAAGAACTGTTCA |
| | | | CCGGCGTGGTGCCCATTCTGGTGGAA |
| | | | CTGGATGGGGATGTGAACGGCCACAA |
| | | | GTTCTCCGTGTCTGGCGAAGGCGAAG |
| | | | GGGATGCCACATACGGCAAGCTGACC |
| | | | CTGAAGTTCATCTGCACCACCGGAAA |
| | | | GCTGCCCGTGCCTTGGCCTACACTGG |
| | | | TCACCACACTGACATACGGCGTGCAG |
| | | | TGCTTCAGCAGATACCCCGACCATAT |
| | | | GAAGCAGCACGACTTCTTCAAGAGCG |
| | | | CCATGCCTGAGGGCTACGTGCAAGAG |
| | | | AGAACCATCTTCTTTAAGGACGACGG |
| | | | CAACTACAAGACCAGGGCCGAAGTGA |
| | | | AGTTCGAGGGCGACACCCTGGTCAAC |
| | | | AGAATCGAGCTGAAGGGCATCGACTT |
| | | | CAAAGAGGATGGCAACATCCTGGGCC |
| | | | ACAAGCTCGAGTACAACTACAACAGC |
| | | | CACAACGTGTACATCATGGCCGACAA |
| | | | GCAGAAAAACGGCATCAAAGTGAACT |
| | | | TCAAGATCCGGCACAACATCGAGGAC |
| | | | GGAAGCGTGCAGCTGGCCGATCACTA |
| | | | CCAGCAGAACACACCTATCGGCGACG |
| | | | GACCTGTGCTGCTGCCTGATAACCAC |
| | | | TACCTGAGCACACAGAGCGCCCTGAG |
| | | | CAAGGACCCTAACGAGAAGAGGGACC |
| | | | ACATGGTGCTGCTGGAATTTGTGACC |
| | | | GCCGCTGGCATCACACTCGGCATGGA |
| | | | CGAGCTGTACAAATGA |
| 91 | Artificial | C-tag | EPEA |
| 92 | Hepatitis B | Hepatitis core | MDIDPYKEFGASVELLSFLPSDFFPS |
| | virus | protein | IRDLLDTASALYREALESPEHVSPHH |
| | | | TALRQAILCWGELMNLATWVGSNLED |
| | | | PASRELVVSYVNVNMGLKLRQILWFH |
| | | | ISCLTFGRETVLEYLVSFGVWIRTPT |
| | | | AYRPPNAPILSTLPETTVVGGGGGSP |
| | | | GGGTPSPGGGGSQSPGGGGSQSGESQ |
| | | | C |
| 93 | Artificial | Tandem | MDIDPYKEFGATVELLSFLPSDFFPS |
| | | hepatitis core | VRDLLDTASALYREALESPEHCSPHH |
| | | protein | TALRQAILCWGELMTLATWVGNNLED |
| | | | PASRDLVVNYVNTNMGLKIRQLLWFH |
| | | | ISCLTFGRETVLEYLVSFGVWIRTPP |
| | | | AYRPPNAPILSTLPETTVVGGSGGSG |
| | | | GSGGSGGSMDIDPYKEFGATVELLSF |
| | | | LPSDFFPSVRDLLDTASALYREALES |
| | | | PEHCSPHHTALRQAILCWGELMTLAT |
| | | | WVGNNLED |
| 94 | Artificial | eGFP | MVSKGEELFTGVVPILVELDGDVNGH |
| | | | KFSVSGEGEGDATYGKLTLKFICTTG |
| | | | KLPVPWPTLVTTLTYGVQCFSRYPDH |
| | | | MKQHDFFKSAMPEGYVQERTIFFKDD |
| | | | GNYKTRAEVKFEGDTLVNRIELKGID |
| | | | FKEDGNILGHKLEYNYNSHNVYIMAD |
| | | | KQKNGIKVNFKIRHNIEDGSVQLADH |
| | | | YQQNTPIGDGPVLLPDNHYLSTQSAL |
| | | | SKDPNEKRDHMVLLEFVTAAGITLGM |
| | | | DELYK |
| 95 | Artificial | His purification | HHHHHH |
| | | tag | |
| 96 | Plasmodium | Plasmodium | KVTVDTVCKRGFLIQMSGHLECKCEN |
| | falciparum | falciparum | DLVLVNEETCEEKVLKCDEKTVNKPC |
| | | antigen | GDFSKCIKIDGNPVSYACKCNLGYDM |
| | | | VNNVCIPNECKQVTCGNGKCILDTSN |
| | | | PVKTGVCSCNIGKVPNVQDQNKCSKD |
| | | | GETKCSLKCLKEQETCKAVDGIYKCD |
| | | | CKDGFIIDQESSICT |
| 97 | Natronobacterium | 2-oxo acid | KAAAEEKAAPAAAKPATTEGEFPETR |
| | gregoryi | dehydrogenase | EKMSGIRRAIAKAMVHSKHTAPHVTL |
| | | subunit E2 | MDEADVTKLVAHRKKEKAIAAEKGIK |
| | | | LTFLPYVVKALVSALREYPVLNTSID |
| | | | DETEEIIQKHYYNIGIAADTDRGLLV |
| | | | PVIKHADRKPIFALAQEINELAEKAR |
| | | | DGKLTPGEMKGASCTITNIGSAGGQW |
| | | | FTPVINHPEVAILGIGRIAEKPIVRD |
| | | | GEIVAAPMLALSLSFDHRMIDGATAQ |
| | | | KALNHIKRLLSDPELLLMEA |
| 98 | Norovirus | Norovirus | KMASSDANPSDGSAANLVPEVNNEVM |
| | | capsid protein | ALEPVVGAAIAAPVAGQQNVIDPWIR |
| | | | NNFVQAPGGEFTVSPRNAPGEILWSA |
| | | | PLGPDLNPYLSHLARMYNGYAGGFEV |
| | | | QVILAGNAFTAGKVIFAAVPPNEPTE |
| | | | GLSPSQVTMFPHIVVDVRQLEPVLIP |
| | | | LPDVRNNFYHYNQSNDPTIKLIAMLY |
| | | | TPLRANNAGDDVFTVSCRVLTRPSPD |
| | | | FDFIFLVPPTVESRTKPFSVPVLTVE |
| | | | EMTNSRFPIPLEKLFTGPSSAFVVQP |
| | | | QNGRCTTDGVLLGTTQLSPVNICTER |
| | | | GDVTHITGSRNYTMNLASQNWNDYDP |
| | | | TEEIPAPLGTPDFVGKIQGVLTQTTR |
| | | | TDGSTRGHKATVYTGSADFAPKLGRV |
| | | | QFETDTDRDFEANQNTKFTPVGVIQD |
| | | | GGTTHRNEPQQWVLPSYSGRNTHNVH |
| | | | LAPAVAPTFPGEQLLFFRSTMPGCSG |
| | | | YPNMDLDCLLPQEWVQYFYQEAAPAQ |
| | | | SDVALLRFVNPDTGRVLFECKLHKSG |
| | | | YVTVAHTGQHDLVIPPNGYFREDSWV |
| | | | NQFYTLAPMGNGTGRRRAV |
| 99 | Homo sapiens | Sign7 | MKRGLCCVLLLCGAVFVSPSQEIHAR |
| | | | FRRA |
| 100 | Homo sapiens | Sign8 | MAFLWLLSCWALLGTTFG |
| 101 | Homo sapiens | Sign9 | MLLLLLLLGLRLQLSLG |

### Items

1. A composition for use in the prophylaxis and/or treatment of a disease wherein the composition comprises:
   i. a first polynucleotide encoding a protein fused to a first peptide tag; and
   ii. a second polynucleotide encoding an antigen fused to a second peptide tag,
wherein the antigen and the protein upon expression in a cell are linked via an isopeptide bond between the first peptide tag and the second peptide tag, whereby i-ii form a particle displaying said antigen.
2. The composition for the use according to item 1, wherein the protein is a particle-forming protein, such as a viral capsid protein or a viral envelope protein such as a glycoprotein.
3. The composition for the use according to item 2, wherein the protein is a protein from a hepatitis virus such as hepatitis B or E, for example a core protein from hepatitis B virus; a protein from a novovirus such as NoV; a protein from a papilloma virus such as Human Papilloma Virus (HPV), preferably HPV16 or HPV18, such as HPV L1; a protein from a polyomavirus such as polyomavirus vp1 (PyV); a protein from a calicivirus such as feline calicivirus (FCV), preferably FCV VP1; a protein from a circovirus such as a porcine circovirus (PCV), preferably PCV2 ORF2; a protein from a nervous necrosis virus (NNV), such as NNV coat protein; a protein from a parvovirus such as canine parvovirus (CVP), preferably CPV VP2, goose parvovirus (GPV) or porcine parvovirus (PPV), preferably structural proteins from GPV or PPV, or parvovirus B19; a protein from a protoparvovirus such as an enteritis virus, for example mink enteritis virus (MEV), preferably MEV VP2, or duck plague virus (DPV), preferably a DPV structural protein.
4. The composition for the use according to any one of the preceding items, wherein the protein is a bacteriophage protein, such as a protein from Salmonella virus P22, from MS2, from QBeta, PRR1, PP7, bacteriophage R17, bacteriophage fr, bacteriophage GA, bacteriophage SP, bacteriophage M11, bacteriophage MX1, bacteriophage NL95, bacteriophage f2 or Cb5.
5. The composition for the use according to any one of the preceding items, wherein the first and/or second peptide tag is derived from the splitting of a protein containing an intramolecular isopeptide bond, identified from a library by using a known binding partner, or designed in silico, to obtain complementary binding partners each containing a reactive amino acid involved in isopeptide bond formation, preferably wherein the first or second peptide tag comprises a tag selected from the group consisting of a SpyTag (SEQ ID NO: 1), a SdyTag (SEQ ID NO: 2), a SnoopTag (SEQ ID NO: 3), a PhoTag (SEQ ID NO: 4), an EntTag (SEQ ID NO: 5), a KTag, a BacTag (SEQ ID NO: 15), a Bac2Tag (SEQ ID NO: 16), a Bac3Tag (SEQ ID NO: 17), a Bac4Tag (SEQ ID NO: 18), a RumTrunkTag (SEQ ID NO: 13 or SEQ ID NO: 14), a Rum7Tag (SEQ ID NO: 12), a RumTag (SEQ ID NO: 6), a Rum2Tag (SEQ ID NO: 7), a Rum3Tag (SEQ ID NO: 8), a Rum4Tag (SEQ ID NO: 9), a Rum5Tag (SEQ ID NO: 10), a Rum6Tag (SEQ ID NO: 11) and a Bac5Tag (SEQ ID NO: 19), and/or preferably wherein the first or second peptide tag comprises a tag selected from the group consisting of a SpyCatcher (SEQ ID NO: 21), a SdyCatcher (SEQ ID NO: 22), a SnoopCatcher (SEQ ID NO: 23) and an esther-forming split-protein pair.
6. The composition for the use according to any one of the preceding items, wherein the disease is cancer, a lipid disorder, a cardiovascular disease, an immune-inflammatory disease, a chronic disease, a neurological disease, an allergic reaction/disease and/or an infectious disease, such as an infectious disease selected from the group consisting of malaria, tuberculosis, and a disease caused by a virus, such as a coronavirus, for example SARS-CoV-2, malaria, tuberculosis, HIV, HCV, Dengue fever, Chikungunya, Yellow fever, HBV or influenza.
7. The composition for the use according to any one of the preceding items, wherein said antigen is a protein, peptide and/or an antigenic fragment from the group consisting of cancer-specific polypeptides, polypeptides associated with cardiovascular diseases, polypeptides associated with asthma, polypeptides associated with nasal polyposis, polypeptides associated with atopic dermatitis, polypeptides associated with eosinophilic esophagitis, polypeptides associated with hypereosinophilic syndrome, polypeptides associated with Churg-Strauss syndrome and polypeptides associated with pathogenic organisms, preferably wherein the antigen is a protein, peptide and/or an antigenic fragment of Severe acute respiratory syndrome coronavirus 2 (SARS-CoV-2).
8. The composition for the use according to any one of the preceding items, wherein the antigen is selected from the group consisting of: hemagglutinin, GD2, EGF-R, CEA, CD52, CD21, neuraminidase, human melanoma protein gp100, human melanoma protein melan-A/MART1, HIV envelope protein, M2e, VAR2CSA, ICAM1, CSP, Dengue virus NS1, Dengue virus envelope protein, Chikungunya virus envelope protein, tyrosinase, HCV E2, NA17-A nt, MAGE-3, HPV 16 E7, HPV L2, PD1, PD-L1, CTLA-4, p53, hCG, Fel d1, EGRFvIII, endoglin, ANGPTL-3, CSPG4, CTLA-4, HER2, IgE, IL-1 beta, IL-5, IL-13, IL-17, IL-22, IL-31, IL-33, TSLP, NGF, (IHNV) G-protein, a lymphotoxin such as lymphotoxin α or β, a lymphotoxin receptor, a receptor activator of nuclear factor kB ligand, vascular endothelial growth factor VEGF, a VEGF receptor, IL-23 p19, ghrelin, CCL21, CXCL12, SDF-1, M-CSF, MCP-1, endoglin, GnRH, TRH, eotaxin, bradykinin, BLC, TNF-α, amyloid β peptide A, angiotensin, gastrin, progastrin, CETP, CCR5, C5a, CXCR4, Des-Arg-bradykinin, GnRH peptide, angiotensin peptide, TNF peptide, or an antigenic fragment or antigenic variant thereof, preferably wherein the antigen is selected from the group consisting of a SARS-CoV-2 envelope protein, a SARS-CoV-2 spike protein, a SARS-CoV-2 nucleocapsid protein and a SARS-CoV-2 envelope protein.
9. A composition according to any one of the preceding items.
10. An expression system comprising:
   i. a first polynucleotide encoding a protein fused to a first peptide tag; and
   ii. a second polynucleotide encoding an antigen fused to a second peptide tag,
wherein upon expression of the first and second polynucleotides in a cell, the antigen and the protein are linked via an isopeptide bond between the first peptide tag and the second peptide tag, whereby i-ii form a particle displaying said antigen, optionally wherein the first peptide tag, the second peptide tag, the protein and/or the antigen are as defined in any one of items 1 to 8.
11. A cell expressing:
   i. a first polynucleotide encoding a protein fused to a first peptide tag, preferably as defined in any one of the preceding items; and
   ii. a second polynucleotide encoding an antigen fused to a second peptide tag, preferably as defined in any one of the preceding items;
wherein the antigen and the protein upon expression in a cell are linked via an isopeptide bond between the first peptide tag and the second peptide tag, whereby i-ii form a particle displaying said antigen, optionally wherein the cell comprises the expression system according to item 10.
12. A host cell, wherein the host cell comprises an expression system according to item 10.
13. The composition for the use according to any one of items 1 to 8, the expression system according to item 10, and/or the cell or host cell according to any one of items 11 to 12, for use in a method for inducing an immune response in a subject, the method comprising the step of administering said composition, polynucleotide or expression system to said subject at least once.
14. A method of administering a composition for use in the prophylaxis and/or treatment of a disease in a subject in need thereof comprising the steps of
   i. obtaining at least one composition as defined in any one of items 1 to 9; and
   ii. administering said composition to a subject at least once for prophylaxis and/or treatment of a disease as defined in any one of the preceding items.
15. A kit of parts comprising
   i. a composition as defined in any one of items 1 to 9 or an expression system according to item 10; and
   ii. optionally, a medical instrument or other means for administering the composition; and
   iii. instructions for use.

1. A composition for use in the prophylaxis and/or treatment of a disease wherein the composition comprises:
i. a first polynucleotide encoding a protein fused to a first peptide tag; and
ii. a second polynucleotide encoding an antigen fused to a second peptide tag,
wherein the antigen and the protein upon expression in a cell are linked via an isopeptide bond, or an ester bond, between the first peptide tag and the second peptide tag, whereby i-ii form a particle displaying said antigen.

2. The composition for the use according to claim 1, wherein the particle is a virus-like particle.

3. The composition for the use according to any one of the preceding claims, wherein the particle is a nanoparticle.

4. The composition for the use according to any one of the preceding claims, wherein the first and/or second polynucleotide is DNA.

5. The composition for the use according to any one of the preceding claims, wherein the first and/or second polynucleotide is RNA.

6. The composition for the use according to claim 5, wherein the RNA is formulated in a lipid particle formulation.

7. The composition for the use according to any one of the preceding claims, wherein the protein is a particle-forming protein.

8. The composition for the use according to any one of the preceding claims, wherein the protein is a viral capsid protein or a viral envelope protein such as a glycoprotein.

9. The composition for the use according to any one of the preceding claims, wherein the protein is a viral capsid protein or a viral envelope protein from a mammalian virus such as a human virus.

10. The composition for the use according to claim 9, wherein the protein is a protein from a hepatitis virus such as hepatitis B or E, for example a core protein from hepatitis B virus; a protein from a norovirus such as NoV; a protein from a papilloma virus such as Human Papilloma Virus (HPV), preferably HPV16 or HPV18, such as HPV L1; a protein from a polyomavirus such as polyomavirus vp1 (PyV); a protein from a calicivirus such as feline calicivirus (FCV), preferably FCV VP1; a protein from a circovirus such as a porcine circovirus (PCV), preferably PCV2 ORF2; a protein from a nervous necrosis virus (NNV), such as NNV coat protein; a protein from a parvovirus such as canine parvovirus (CVP), preferably CPV VP2, goose parvovirus (GPV) or porcine parvovirus (PPV), preferably structural proteins from GPV or PPV, or parvovirus B19; a protein from a protoparvovirus such as an enteritis virus, for example mink enteritis virus (MEV), preferably MEV VP2, or duck plague virus (DPV), preferably a DPV structural protein.

11. The composition for the use according to any one of the preceding claims, wherein the protein is a protein from a plant virus, such as a cowpea virus, a tobacco virus, a tomato virus, a cucumber virus or a potato virus.

12. The composition for the use according to claim 11, wherein the plant virus is a mosaic virus, preferably Cowpea mosaic virus (CPMV); tobacco mosaic virus (TMV); tomato spotted wilt virus (TSWV); tomato yellow leaf curl virus (TYLCV); cucumber mosaic virus (CMV); or from potato virus Y (PVY).

13. The composition for the use according to any one of the preceding claims, wherein the protein is a bacteriophage protein, such as a protein from Salmonella virus P22, from MS2, from QBeta, PRR1, PP7, bacteriophage R17, bacteriophage fr, bacteriophage GA, bacteriophage SP, bacteriophage M11, bacteriophage MX1, bacteriophage NL95, bacteriophage f2 or Cb5.

14. The composition for the use according to any one of the preceding claims, wherein the protein is a particle forming protein, such as ferritin as set forth in SEQ ID NO: 69, i301 as set forth in SEQ ID NO: 72, replicase polyprotein 1a (pp1a), a lumazine synthase as set forth in SEQ ID NO: 70, Hbc as set forth in SEQ ID NO: 92, tandemHBc as set forth in SEQ ID NO: 93, the 2-oxo acid dehydrogenase subunit E2 as set forth in SEQ ID NO: 97, or the norovirus capsid protein as set forth in SEQ ID NO: 98.

15. The composition for the use according to any one of the preceding claims, wherein the first or second peptide tag comprises a tag selected from the group consisting of a SpyTag as set forth in SEQ ID NO: 1, a SdyTag as set forth in SEQ ID NO: 2, a SnoopTag as set forth in SEQ ID NO: 3, a PhoTag as set forth in SEQ ID NO: 4, an EntTag as set forth in SEQ ID NO: 5, a KTag, a BacTag as set forth in SEQ ID NO: 15, a Bac2Tag as set forth in SEQ ID NO: 16, a Bac3Tag as set forth in SEQ ID NO: 17, a Bac4Tag as set forth in SEQ ID NO: 18, a RumTrunk D9N tag as set forth in SEQ ID NO: 13 or SEQ ID NO: 14, a Rum7Tag as set forth in SEQ ID NO: 12, a RumTag as set forth in SEQ ID NO: 6, a Rum2Tag as set forth in SEQ ID NO: 7, a Rum3Tag as set forth in SEQ ID NO: 8, a Rum4Tag as set forth in SEQ ID NO: 9, a Rum5Tag as set forth in SEQ ID NO: 10, a Rum6Tag as set forth in SEQ ID NO: 11 and a Bac5Tag as set forth in SEQ ID NO: 19.

16. The composition for the use according to any one of the preceding claims, wherein the first or second peptide tag comprises a tag selected from the group consisting of a SpyCatcher as set forth in SEQ ID NO: 21, a SdyCatcher as set forth in SEQ ID NO: 22, a SnoopCatcher as set forth in SEQ ID NO: 23 and an esther-forming split-protein pair.

17. The composition for the use according to any one of the preceding claims, wherein the composition further comprises a SpyLigase, whereby the two peptide tags may be locked together.

18. The composition for the use according to any one of the preceding claims, wherein one of the first and second peptide tags is an SdyTag and the other of the first and second peptide tags is an SdyCatcher.

19. The composition for the use according to any one of the preceding claims, wherein one of the first and second peptide tags is a SpyTag, such as the SpyTag as set forth in SEQ ID NO: 1 or a homologue thereof having at least 70%, such as at least 80%, such as at least 90% sequence identity thereto, and the other of the first and second peptide tags is a SpyCatcher, such as the SpyCatcher as set forth in SEQ ID NO: 21 or a homologue thereof having at least 70%, such as at least 80%, such as at least 90% sequence identity thereto.

20. The composition for the use according to any one of the preceding claims, wherein one of the first and second peptide tags is a SnoopTag and the other of the first and second peptide tags is a SnoopCatcher.

21. The composition for the use according to any one of the preceding claims, wherein one or more first peptide tags are fused to the N-terminal end, to the C-terminal end of the protein and/or inserted in-frame into the coding sequence of the protein, optionally by a linker.

22. The composition for the use according to any one of the preceding claims, wherein one or more second peptide tags are fused to the N-terminal end, to the C-terminal end of the antigen and/or inserted in-frame into the coding sequence of the antigen, optionally by a linker.

23. The composition for the use according to any one of the preceding claims, wherein the disease is cancer, a lipid disorder, a cardiovascular disease, an immune-inflammatory disease, a chronic disease, a neurological disease, an infectious disease and/or an allergic reaction/disease.

24. The composition for the use according to claim 23, wherein the cancer is selected from the group consisting of breast cancer, gastric cancer, ovarian cancer, glioblastoma, bone cancer, skin cancer and uterine serous carcinoma.

25. The composition for the use according to any one of claims 23 to 24, wherein the lipid disorder is selected from the group consisting of a lipid disorder such as hyperlipidemia, type I, type II, type III, type IV, or type V hyperlipidemia, secondary hypertriglyceridemia, hypercholesterolemia, familial hypercholesterolemia, xanthomatosis and cholesterol acetyltransferase deficiency.

26. The composition for the use according to any one of claims 23 to 25, wherein the cardiovascular disease is selected from the group consisting of dyslipidemia, atherosclerosis, a coronary artery disease and/or hypercholesterolemia.

27. The composition for the use according to any one of claims 23 to 26, wherein the chronic or immune-inflammatory disease is selected from the group consisting of eosinophilic asthma, allergy, nasal polyposis, atopic dermatitis, eosinophilic esophagitis, hypereosinophilic syndrome, contact allergy and Churg-Strauss syndrome.

28. The composition for the use according to any one of claims 23 to 27, wherein the neurological disease is Alzheimer's disease.

29. The composition for the use according to any one of claims 23 to 28, wherein the infectious disease is selected from the group consisting of malaria, tuberculosis, and a disease caused by a virus, such as a coronavirus, for example SARS-CoV-2, malaria, tuberculosis, HIV, HCV, Dengue fever, Chikungunya, Yellow fever, HBV or influenza.

30. The composition for the use according to any one of the preceding claims, wherein the antigen is a polypeptide, peptide and/or an antigenic fragment of a polypeptide associated with or specific for an abnormal physiological response.

31. The composition for the use according to claim 30, wherein the abnormal physiological response is an autoimmune disease, an allergic reaction and/or a cancer.

32. The composition for the use according to any one of the preceding claims, wherein said antigen is a protein, peptide and/or an antigenic fragment from the group consisting of cancer-specific polypeptides, polypeptides associated with cardiovascular diseases, polypeptides associated with asthma, polypeptides associated with nasal polyposis, polypeptides associated with atopic dermatitis, polypeptides associated with eosinophilic esophagitis, polypeptides associated with hypereosinophilic syndrome, polypeptides associated with Churg-Strauss syndrome and polypeptides associated with pathogenic organisms.

33. The composition for the use according to any one of the preceding claims, wherein said antigen is a protein, peptide and/or an antigenic fragment of Severe acute respiratory syndrome coronavirus 2 (SARS-CoV-2).

34. The composition for the use according to any one of the preceding claims, wherein the antigen is selected from the group consisting of: hemagglutinin, GD2, EGF-R, CEA, CD52, CD21, neuraminidase, human melanoma protein gp100, human melanoma protein melan-A/MART1, HIV envelope protein, M2e, VAR2CSA, ICAM1, CSP, Dengue virus NS1, Dengue virus envelope protein, Chikungunya virus envelope protein, tyrosinase, HCV E2, NA17-A nt, MAGE-3, HPV 16 E7, HPV L2, PD1, PD-L1, CTLA-4, p53, hCG, Fel d1, EGRFvIII, endoglin, ANGPTL-3, CSPG4, CTLA-4, HER2, IgE, IL-1 beta, IL-5, IL-13, IL-17, IL-22, IL-31, IL-33, TSLP, NGF, (IHNV) G-protein, a lymphotoxin such as lymphotoxin α or β, a lymphotoxin receptor, a receptor activator of nuclear factor kB ligand, vascular endothelial growth factor VEGF, a VEGF receptor, IL-23 p19, ghrelin, CCL21, CXCL12, SDF-1, M-CSF, MCP-1, endoglin, GnRH, TRH, eotaxin, bradykinin, BLC, TNF-α, amyloid β peptide A, angiotensin, gastrin, progastrin, CETP, CCR5, C5a, CXCR4, Des-Arg-bradykinin, GnRH peptide, angiotensin peptide, TNF peptide, or an antigenic fragment or antigenic variant thereof.

35. The composition for the use according to any one of the preceding claims, wherein the antigen is selected from the group consisting of a SARS-CoV-2 envelope protein, a SARS-CoV-2 spike protein, a SARS-CoV-2 nucleocapsid protein and a SARS-CoV-2 envelope protein.

36. The composition for the use according to any one of the preceding claims, wherein the antigen is selected from the group consisting of IL-13, IL-31, IL-17A, PCSK9, an HIV trimer and an influenza virus trimer.

37. The composition for the use according to any one of the preceding claims, wherein the antigen is selected from the group consisting of eGFP as set forth in SEQ ID NO: 94 or a homologue thereof having at least 70%, such as at least 80%, such as at least 90% sequence identity thereto, Pfs25 as set forth in SEQ ID NO: 96 or a homologue thereof having at least 70%, such as at least 80%, such as at least 90% sequence identity thereto, and the His purification tag as set forth in SEQ ID NO: 95 or a homologue thereof having at least 70%, such as at least 80%, such as at least 90% sequence identity thereto.

38. The composition for the use according to any one of the preceding claims for use in treatment of an infectious disease, wherein the antigen is a protein or a peptide specific to the pathogenic organism which causes the infectious disease, or a fragment thereof.

39. The composition for the use according to any one of the preceding claims, wherein the antigen is capable of eliciting an immune reaction in an animal, such as a mammal, such as a Homo sapiens, cow, pig, horse, sheep, goat, llama, mouse, rat, monkey, and/or a bird, such as a chicken and/or a fish, such as a salmon.

40. The composition for the use according to any one of the preceding claims, wherein the antigen further comprises a polyhistidine tag.

41. A composition as defined in any one of the preceding claims, wherein the protein is selected from the group consisting of i301 as set forth in SEQ ID NO: 72 or a homologue thereof having at least 70%, such as at least 80%, such as at least 90% sequence identity thereto, Hbc as set forth in SEQ ID NO: 92 or a homologue thereof having at least 70%, such as at least 80%, such as at least 90% sequence identity thereto, tandemHBc as set forth in SEQ ID NO: 93 or a homologue thereof having at least 70%, such as at least 80%, such as at least 90% sequence identity thereto, human ferritin as set forth in SEQ ID NO: 69 or a homologue thereof having at least 70%, such as at least 80%, such as at least 90% sequence identity thereto, the 2-oxo acid dehydrogenase subunit E2 as set forth in SEQ ID NO: 97 or a homologue thereof having at least 70%, such as at least 80%, such as at least 90% sequence identity thereto, lumazine synthase as set forth in SEQ ID NO: 70 or a homologue thereof having at least 70%, such as at least 80%, such as at least 90% sequence identity thereto, and the norovirus capsid protein as set forth in SEQ ID NO: 98 or a homologue thereof having at least 70%, such as at least 80%, such as at least 90% sequence identity thereto.

42. The composition according to claim 41, wherein the first and the second peptide tags are selected from the group consisting of SpyTag as set forth in SEQ ID NO: 1 or a homologue thereof having at least 70%, such as at least 80%, such as at least 90% sequence identity thereto, and SpyCatcher as set forth in SEQ ID NO: 21 or a homologue thereof having at least 70%, such as at least 80%, such as at least 90% sequence identity thereto.

43. The composition according to any one of claims 41 to 42, wherein the antigen is selected from the group consisting of eGFP as set forth in SEQ ID NO: 94 or a homologue thereof having at least 70%, such as at least 80%, such as at least 90% sequence identity thereto, Pfs25 as set forth in SEQ ID NO: 96 or a homologue thereof having at least 70%, such as at least 80%, such as at least 90% sequence identity thereto, and the His purification tag as set forth in SEQ ID NO: 95 or a homologue thereof having at least 70%, such as at least 80%, such as at least 90% sequence identity thereto.

44. An expression system comprising:
i. a first polynucleotide encoding a protein fused to a first peptide tag; and
ii. a second polynucleotide encoding an antigen fused to a second peptide tag,
wherein upon expression of the first and second polynucleotides in a cell, the antigen and the protein are linked via an isopeptide bond, or an ester bond, between the first peptide tag and the second peptide tag, whereby i-ii form a particle displaying said antigen.

45. The expression system according to claim 44, wherein the first peptide tag, the second peptide tag, the protein and/or the antigen are as defined in any one of claims 1 to 40.

46. The expression system according to any one of claim 44, wherein the first polynucleotide and the second polynucleotide are both DNA polynucleotides or RNA polynucleotides.

47. The expression system according to any one of claims 44 to 46, wherein the first and second polynucleotides are comprised within one vector such as a viral vector or a plasmid, or wherein the first and second polynucleotides are comprised within two vectors such as two viral vectors; two plasmids; or one viral vector and one plasmid.

48. The expression system according to claim 47, wherein the viral vector is an adenoviral vector, such as a modified adenoviral vector, e.g. the replication-deficient simian adenovirus vector ChAdOx1, or a modified vaccinia Ankara (MVA) vector.

49. The expression system according to any one of claims 44 to 47, wherein the expression system comprises or consists of a plasmid.

50. The expression system according to claim 49, wherein said plasmid is pVAX1.

51. The expression system according to any one of claims 44 to 50, wherein the expression system comprises or consists of an mRNA.

52. The expression system according to any one of claims 44 to 51, wherein the first polynucleotide comprises or consists of a first polynucleotide encoding a protein comprising a first peptide tag, and the second polynucleotide comprises or consists of a second polynucleotide encoding an antigen comprising a second peptide tag.

53. The expression system according to any one of claims 44 to 52, wherein the first polynucleotide and the second polynucleotide are comprised within the same nucleic acid molecule, or within two different nucleic acid molecules.

54. The expression system according to any one of claims 44 to 53, further comprising a first promoter upstream of the first polynucleotide.

55. The expression system according to any one of claims 44 to 54, further comprising a second promoter upstream of the second polynucleotide.

56. The expression system according to any one of claims 44 to 55, wherein the first promoter is a constitutive promoter and the second promoter is an inducible promoter, such as a vitamin D-inducible promoter.

57. The expression system according to any one of claims 44 to 55, wherein the first and the second promoters are constitutive promoters.

58. The expression system according to any one of claims 44 to 57, wherein the first polynucleotide further comprises a secretion or excretion signal, whereby the protein fused to the first peptide tag is secreted or excreted from the endoplasmic reticulum of the cell.

59. The expression system according to any one of claims 44 to 58, wherein the second polynucleotide further comprises a secretion or excretion signal, whereby the antigen fused to the second peptide tag is secreted or excreted from the endoplasmic reticulum of the cell.

60. The expression system according to any one of claims 44 to 59, wherein the secretion or excretion signal comprises or consists of SEQ ID NO: 79, SEQ ID NO: 80, SEQ ID NO: 81, SEQ ID NO: 82, SEQ ID NO: 83 or SEQ ID NO: 84.

61. A cell expressing:
i. a first polynucleotide encoding a protein fused to a first peptide tag, preferably as defined in any one of the preceding claims; and
ii. a second polynucleotide encoding an antigen fused to a second peptide tag, preferably as defined in any one of the preceding claims;
wherein the antigen and the protein upon expression in a cell are linked via an isopeptide bond, or an ester bond, between the first peptide tag and the second peptide tag, whereby i-ii form a particle displaying said antigen.

62. The cell according to claim 61, comprising the expression system according to any one of claims 44 to 60.

63. A host cell, wherein the host cell comprises an expression system according to any one of claims 44 to 60.

64. The host cell according to claim 63, wherein the host cell is selected from the group consisting of a bacterial cell, a yeast cell, a fungal cell, a plant cell, a mammalian cell and an insect cell.

65. The composition for the use according to any one of claims 1 to 40, the expression system according to any one of claims 44 to 60, and/or the cell or host cell according to any one of claims 61 to 64, for use in a method for inducing an immune response in a subject, the method comprising the step of administering said composition, polynucleotide or expression system to said subject at least once.

66. A method of administering a composition for use in the prophylaxis and/or treatment of a disease in a subject in need thereof comprising the step of
i. administering at least one composition as defined in any one of claims 1 to 40 to a subject at least once for prophylaxis and/or treatment of a disease as defined in any one of the preceding claims.

67. The method of administering a composition for the use according to claim 66, wherein the composition is boosted by administration in a form or body part different from the previous administration.

68. The method of administering a composition for the use according to any one of claims 66 to 67, wherein the composition is administered to the area most likely to be the receptacle of a given disease.

69. The method of administering a composition for the use according to any one of claims 66 to 68, wherein the subject is an animal, such as a mammal, such as a cow, pig, horse, sheep, goat, llama, mouse, rat, monkey, most preferably such as a human being; or a bird, such as a chicken or a fish, such as a salmon.

70. The method of administering a composition for the use according to any one of claims 66 to 69, wherein the composition is administered in combination with any other vaccine.

71. The method of administering a composition for the use according to any one of claims 66 to 70, wherein the composition forms a part of a vaccine cocktail.

72. A kit of parts comprising
i. a composition as defined in any one of the preceding claims or an expression system according to any one of claims 44 to 60, and
ii. optionally, a medical instrument or other means for administering the composition, and
iii. instructions for use.

73. The kit of parts according to claim 72, comprising a second active ingredient.


SEQUENCE LISTING (101 sequences)

SEQ ID NO: 1 - protein, 13 aa, Streptococcus pyogenes
AHIVMVDAYKPTK

SEQ ID NO: 2 - protein, 13 aa, Streptococcus dysgalactiae
DPIVMIDNDKPIT

SEQ ID NO: 3 - protein, 12 aa, Streptococcus pneumoniae
KLGDIEFIKVNK

SEQ ID NO: 4 - protein, 22 aa, Streptococcus phocae
LVTGTAHIVMVDNYKPIVETGD

SEQ ID NO: 5 - protein, 15 aa, Enterococcus faecalis
NTIVMVDKLKEVPPT

SEQ ID NO: 6 - protein, 20 aa, Ruminococcus sp. AF26-25AA
SENGNPLIVMVDDTTKVKIS

SEQ ID NO: 7 - protein, 17 aa, Ruminococcus sp. AF25-19
GTPIVIMVDEAKPSLPD

SEQ ID NO: 8 - protein, 17 aa, Ruminococcus sp. Marseille-P6503
GNPLIVMIDEAEQKEIP

SEQ ID NO: 9 - protein, 17 aa, Ruminococcus sp.
AGGIIVMKDNTTKVSIS

SEQ ID NO: 10 - protein, 17 aa, Ruminococcus flavefaciens
GNPIVTMIDDATLVKIS

SEQ ID NO: 11 - protein, 17 aa, Ruminococcus sp.
GNSTITMVDDTTKVHIT

SEQ ID NO: 12 - protein, 17 aa, Ruminococcus sp. AM43-6
GTPLIVMVDDTTKVEIS

SEQ ID NO: 13 - protein, 15 aa, Artificial sequence
  Rumtrunk D9N
  MISC_FEATURE (1)..(15): Rumtrunk D9N
GNPLIVMVNDTTKVK

SEQ ID NO: 14 - protein, 15 aa, Ruminococcus sp.
GNPLIVMVDDTTKVK

SEQ ID NO: 15 - protein, 20 aa, Bacillus cereus
NEKVTGQFEIVKVDANDKTK

SEQ ID NO: 16 - protein, 19 aa, Bacillus cereus
SKSLGQFEIVKVDAQDKTK

SEQ ID NO: 17 - protein, 16 aa, Bacillus cereus
LGQFEIVKVDSQDKTK

SEQ ID NO: 18 - protein, 17 aa, Bacillus cereus
VTGQFEIVKVDAEDKTR

SEQ ID NO: 19 - protein, 19 aa, Bacillus cereus VD022
EKVMGQFEIMKVDANDKTK

SEQ ID NO: 20 - protein, 23 aa, Clostridium perfringens B str. ATCC 3626
DTKQVVKHEDKNDKAQTLVVEKP

SEQ ID NO: 21 - protein, 116 aa, Streptococcus pyogenes
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 22 - protein, 104 aa, Streptococcus dysgalactiae
IDTMSGLSGETGQSGNTTIEEDSTTHVKFSKRDSNGKELAGAMIELRNLSGQTIQSWVSDGTVKDFYLMPGTYQFVETAAPEGYELAAPITFTIDEKGQIWVDS

SEQ ID NO: 23 - protein, 123 aa, Streptococcus pneumoniae
SSGLVPRGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 24 - protein, 129 aa, Actinomyces viscosus
GSLSKYGKVILTKTGTDDLADKTKYNGAQFQVYECTKTASGATLRDSDPSTQTVDPLTIGGEKTFTTAGQGTVEINYLRANDYVNGAKKDQLTDEDYYCLVETKAPEGYNLQADPLPFRVLAEKAEKKA

SEQ ID NO: 25 - protein, 102 aa, Streptococcus pneumonia serotype 4 (strain ATCC BAA-334/TIGR4)
GSTTKVKLIKVDQDHNRLEGVGFKLVSVARDVSAAAVPLIGEYRYSSSGQVGRTLYTDKNGEIFVTNLPLGNYRFKEVEPLAGYAVTTLDTDVQLVDHQLVT

SEQ ID NO: 26 - protein, 94 aa, Streptococcus pneumonia serotype 4 (strain ATCC BAA-334/TIGR4)
PRGNVDFMKVDGRTNTSLQGAMFKVMKEESGHYTPVLQNGKEVVVTSGKDGRFRVEGLEYGTYYLWELQAPTGYVQLTSPVSFTIGKDTRKELV

SEQ ID NO: 27 - protein, 123 aa, Corynebacterium diphtheriae NCTC 13129
VVTYHGKLKVVKKDGKEAGKVLKGAEFELYQCTSAAVLGKGPLTVDGVKKWTTGDDGTFTIDGLHVTDFEDGKEAAPATKKFCLKETKAPAGYALPDPNVTEIEFTRAKISEKDKFEGDDEVT

SEQ ID NO: 28 - protein, 134 aa, Lactobacillus rhamnosus GG
STNDTTTQNVVLTKYGFDKDVTAIDRATDQIWTGDGAKPLQGVDFTIYNVTANYWASPKDYKGSFDSAPVAATGTTNDKGQLTQALPIQSKDASGKTRAAVYLFHETNPRAGYNTSADFWLTLPAKAAADGNVY

SEQ ID NO: 29 - protein, 114 aa, Lactobacillus rhamnosus GG
TTYERTFVKKDAETKEVLEGAGFKISNSDGKFLKLTDKDGQSVSIGEGFIDVLANNYRLTWVAESDATVFTSDKSGKFGLNGFADNTTTYTAVETNVPDGYDAAANTDFKADNS

SEQ ID NO: 30 - protein, 107 aa, Streptococcus agalactiae A909
GQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNP

SEQ ID NO: 31 - protein, 91 aa, Streptococcus intermedius
EQDVVFSKVNVAGEEIAGAKIQLKDAQGQVVHSWTSKAGQSETVKLKAGTYTFHEASAPTGYLAVTDITFEVDVQGKVTVKDANGNGVKAD

SEQ ID NO: 32 - protein, 104 aa, Streptococcus pneumoniae
KLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQ

SEQ ID NO: 33 - protein, 130 aa, Corynebacterium diphtheriae NCTC 13129
GSERKGSLTLHKKKGAESEKRATGKEMDDVAGEPLNGVTFKITKLNFDLQNGDWAKFPKTAADAKGHETSTTKEVETSGNGTAVFDNLDLGIYLVEETKAPDGIVTGAPFIVSIPMVNEASDAWNYNVVA

SEQ ID NO: 34 - protein, 149 aa, Clostridium perfringens B str. ATCC 3626
NLPEVKDGTLRTTVIADGVNGSSEKEALVSFENSKDGVDVKDTINYEGLVANQNYTLTGTLMHVKADGSLEEIATKTTNVTAGENGNGTWGLDFGNQKLQVGEKYVVFENAESVENLIDTDKDYNLDTKQVVKHEDKNDKAQTLVVEKP

SEQ ID NO: 35 - DNA, 39 bp, Streptococcus pyogenes
gctcacatcgtgatggtggacgcttacaagcccaccaag

SEQ ID NO: 36 - DNA, 39 bp, Streptococcus dysgalactiae
gaccccatcgtgatgatcgacaacgacaagcccatcacc

SEQ ID NO: 37 - DNA, 36 bp, Streptococcus pneumoniae
aaactgggtgatattgaatttattaaagttaataaa

SEQ ID NO: 38 - DNA, 66 bp, Streptococcus phocae
ctggttaccggcaccgcacatattgttatggttgataactataagccgatcgtggaaaccggtgat

SEQ ID NO: 39 - DNA, 60 bp, Enterococcus faecalis
accgaagttagcggtaataccattgtgatggtggataaactgaaagaagttccgcctacc

SEQ ID NO: 40 - DNA, 60 bp, Ruminococcus sp. AF26-25AA
agcgaaaacggcaacccgctgattgtgatggtggatgataccaccaaagtgaaaattagc

SEQ ID NO: 41 - DNA, 51 bp, Ruminococcus sp. AF25-19
ggcaccccgattgtgattatggtggatgaagcgaaaccgagcctgccggat

SEQ ID NO: 42 - DNA, 51 bp, Ruminococcus sp. Marseille-P6503
ggcaacccgctgattgtgatgattgatgaagcggaacagaaagaaattccg

SEQ ID NO: 43 - DNA, 51 bp, Ruminococcus sp.
gcgggcggcattattgtgatgaaagataacaccaccaaagtgagcattagc

SEQ ID NO: 44 - DNA, 48 bp, Ruminococcus flavefaciens
ggcaacccgattgtgaccatgattgatgatgcgaccctggtgaaaatt

SEQ ID NO: 45 - DNA, 51 bp, Ruminococcus sp.
ggcaacagcaccattaccatggtggatgataccaccaaagtgcatattacc

SEQ ID NO: 46 - DNA, 51 bp, Ruminococcus sp. AM43-6
ggcaccccgctgattgtgatggtggatgataccaccaaagtggaaattagc

SEQ ID NO: 47 - DNA, 45 bp, Artificial sequence
  Rumtrunk D9N DNA
  misc_feature (1)..(45): Rumtrunk D9N
ggtaatccgctgattgtgatggtgaatgataccaccaaagtgaaa

SEQ ID NO: 48 - DNA, 45 bp, Ruminococcus sp.
ggcaacccgctgattgtgatggtggatgataccaccaaagtgaaa

SEQ ID NO: 49 - DNA, 45 bp, Bacillus cereus
ggtcagttcgaaattgttaaagttgatgcaaacgataaaactaaa

SEQ ID NO: 50 - DNA, 57 bp, Bacillus cereus
agcaaaagcctgggccagtttgaaattgtgaaagtggatgcgcaggataaaaccaaa

SEQ ID NO: 51 - DNA, 48 bp, Bacillus cereus
ctgggccagtttgaaattgttaaagttgatagccaggataaaaccaaa

SEQ ID NO: 52 - DNA, 51 bp, Bacillus cereus
gttaccggtcagtttgaaatcgttaaagttgatgccgaagataagacccgt

SEQ ID NO: 53 - DNA, 57 bp, Bacillus cereus VD022
gaaaaagtgatgggccagttcgaaatcatgaaagttgatgccaacgacaagaccaaa

SEQ ID NO: 54 - DNA, 69 bp, Clostridium perfringens B str. ATCC 3626
gacaccaagcaggtggtgaagcacgaggacaagaacgacaaggcccagaccctggtggtggagaagccc

SEQ ID NO: 55 - DNA, 348 bp, Streptococcus pyogenes
ggtgcaatggttgataccctgagcggtctgagcagcgaacagggtcagagcggtgatatgaccattgaagaagatagcgcaacccacatcaaattcagcaaacgtgatgaagatggtaaagaactggcaggcgcaacaatggaactgcgtgatagcagcggtaaaaccattagcacctggattagtgatggtcaggtgaaagatttttatctgtaccctggcaaatacacctttgttgaaaccgcagcaccggatggttatgaagttgcaaccgcaattacctttaccgttaatgaacagggccaggttaccgtgaatggtaaagcaaccaaaggtgatgcacatatt

SEQ ID NO: 56 - DNA, 318 bp, Streptococcus dysgalactiae
atgggtattgataccatgagcggtctgagcggtgaaaccggtcagagcggtaataccaccattgaagaggatagcaccacacatgtgaaattcagcaaacgcgatgcaaacggcaaagaactggcaggcgcaatgattgaactgcgtaatctgagtggtcagaccattcagagctgggttagtgatggcaccgttaaagatttttatctgatgcctggcacctatcagtttgttgaaaccgcagcaccggaaggttatgagctggcagcaccgattacctttaccattgatgaaaaaggtcagatttgggttgatagc

SEQ ID NO: 57 - DNA, 369 bp, Streptococcus pneumoniae
agcagcggcctggtgccgcgcggcagccatatgaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcaggatattccggctacatatgaatttaccaacggtaaacattatatcaccaatgaaccgataccgccgaaa

SEQ ID NO: 58 - DNA, 387 bp, Actinomyces viscosus
ggtagcctgagcaaatatggtaaagtgattctgaccaaaaccggcaccgatgatctggcagataaaaccaaatataacggtgcacagtttcaggtgtatgaatgtaccaaaacagcaagcggtgcaaccctgcgtgatagcgatccgagcacacagaccgttgatccgctgaccattggtggtgaaaaaacctttaccaccgcaggtcagggcaccgttgaaattaattatctgcgtgccaatgattatgtgaacggtgcaaaaaaagatcagctgaccgatgaagattattactgtctggttgaaaccaaagcaccggaaggttataatctgcaggcagatccgctgccgtttcgtgttctggccgaaaaagcagaaaaaaaagcc

SEQ ID NO: 59 - DNA, 306 bp, Streptococcus pneumonia serotype 4 (strain ATCC BAA-334/TIGR4)
ggtagcaccaccaaagtgaaactgattaaagttgatcaggatcacaatcgtctggaaggtgttggttttaaactggttagcgttgcacgtgatgttagcgcagcagcagttccgctgattggtgaatatcgttatagcagcagcggtcaggttggtcgtaccctgtataccgataaaaatggcgaaattttcgttaccaatctgccgctgggtaactatcgttttaaagaagttgaaccgctggcaggttatgcagttaccacactggataccgatgttcagctggttgatcatcagctggtgacc

SEQ ID NO: 60 - DNA, 282 bp, Streptococcus pneumonia serotype 4 (strain ATCC BAA-334/TIGR4)
ccgcgtggtaatgttgattttatgaaagttgatggtcgcaccaataccagcctgcagggtgcaatgtttaaagtgatgaaagaagaaagcggtcactatacaccggtgctgcagaatggtaaagaagttgttgttaccagcggtaaagatggtcgttttcgtgttgaaggtctggaatatggcacctattatctgtgggaactgcaggcaccgaccggttatgttcagctgaccagtccggttagttttaccattggcaaagatacccgtaaagaactggtg

SEQ ID NO: 61 - DNA, 369 bp, Corynebacterium diphtheriae NCTC 13129
gttgttacctatcatggtaaactgaaagtggtgaaaaaagacggtaaagaggcaggcaaagttctgaaaggtgcagaatttgaactgtatcagtgtaccagcgcagcagttttaggtaaaggtccgctgaccgttgatggtgtgaaaaaatggaccaccggtgatgatggcacctttaccattgatggtctgcatgttaccgattttgaagatggtaaagaagccgcaccggcaaccaaaaaattctgtctgaaagaaaccaaagcaccggcaggttatgcactgcctgatccgaatgtgaccgaaattgaatttacccgtgcaaaaatcagcgagaaagataaatttgaaggcgacgatgaagtgacc

SEQ ID NO: 62 - DNA, 402 bp, Lactobacillus rhamnosus GG
agcaccaatgataccaccacacagaatgttgttctgaccaaatatggcttcgataaagatgttaccgcaattgatcgtgcaaccgatcagatttggaccggtgatggtgcaaaaccgctgcagggtgttgattttaccatttataacgtgaccgccaattattgggcaagcccgaaagattataaaggcagctttgatagcgcaccggttgcagccaccggtacaacaaatgataaaggccagctgacccaggcactgccgattcagagcaaagatgcaagcggtaaaacccgtgcagcagtttacctgtttcacgaaaccaatccgcgtgcaggttataataccagcgcagatttttggctgaccctgcctgcaaaagcagcagcagatggtaatgtttat

SEQ ID NO: 63 - DNA, 342 bp, Lactobacillus rhamnosus GG
accacctatgaacgtacctttgttaaaaaagacgccgaaaccaaagaagttctggaaggcgcaggctttaaaatcagcaatagtgatggcaaattcctgaaactgaccgataaagatggtcagagcgttagcattggtgaaggttttattgatgttctggccaataactatcgtctgacctgggttgcagaaagtgatgcaaccgtttttaccagcgataaaagcggcaaatttggtctgaatggttttgcagataataccaccacctataccgcagttgaaaccaatgttccggatggttatgatgcagcagcaaacaccgatttcaaagccgataatagc

SEQ ID NO: 64 - DNA, 321 bp, Streptococcus agalactiae A909
ggtcagattaccatcaaaaaaatcgatggtagcaccaaagcaagcctgcagggtgcaatttttgttctgaaaaatgcaaccggtcagttcctgaattttaacgataccaataatgttgaatggggcaccgaagcaaatgccaccgaatataccaccggtgcagatggtattattaccattaccggtctgaaagaaggcacctattacctggttgaaaaaaaagcaccgctgggttataatctgctggataattcacagaaagtgattttaggtgatggtgcaaccgataccaccaatagcgataacctgctggttaatccg

SEQ ID NO: 65 - DNA, 273 bp, Streptococcus intermedius
gaacaggatgttgtgtttagcaaagttaatgttgccggtgaagaaattgcgggtgcaaaaatccagctgaaagatgcacagggtcaagttgttcatagctggaccagcaaagcaggtcagagcgaaaccgttaaactgaaagcaggcacctatacctttcatgaagcaagcgcaccgaccggttatctggcagttaccgatattacctttgaagttgatgttcagggtaaagtgaccgttaaagatgcaaatggtaatggtgtgaaagccgac

SEQ ID NO: 66 - DNA, 312 bp, Streptococcus pneumoniae
aaactgggtgatattgagttcatcaaagtgaacaaaaacgataaaaaaccgctgcgtggtgcagtttttagcctgcagaaacagcatccggattacccggatatttatggtgcaattgatcagaatggcacctatcagaatgttcgtaccggtgaagatggtaaactgacctttaaaaacctgagcgacggtaaatatcgcctgtttgaaaatagcgaaccggcaggttataaaccggttcagaataaaccgattgtggcctttcagattgttaatggtgaagttcgtgatgtgaccagcattgttccgcag

SEQ ID NO: 67 - DNA, 390 bp, Corynebacterium diphtheriae NCTC 13129
ggtagcgaacgtaaaggtagtctgaccctgcataaaaagaaaggtgcagaaagcgaaaaacgtgcaaccggtaaagaaatggatgatgttgccggtgaaccgctgaatggtgttacctttaaaatcaccaaactgaacttcgatctgcagaatggtgattgggcaaaatttccgaaaaccgcagcagatgcaaaaggtcatgaaaccagcaccaccaaagaagtggaaaccagcggtaatggcaccgcagtttttgataatctggatctgggtatttacctggtggaagaaaccaaagcaccggatggtattgttacaggtgcaccgtttattgttagcattccgatggttaatgaagcaagtgatgcctggaattataacgttgttgca

SEQ ID NO: 68 - DNA, 447 bp, Clostridium perfringens B str. ATCC 3626
aacctgcccgaggtgaaggacggcaccctgaggaccaccgtgatcgccgacggcgtgaacggcagcagcgagaaggaggccctggtgagcttcgagaacagcaaggacggcgtggacgtgaaggacaccatcaactacgagggcctggtggccaaccagaactacaccctgaccggcaccctgatgcacgtgaaggccgacggcagcctggaggagatcgccaccaagaccaccaacgtgaccgccggcgagaacggcaacggcacctggggcctggacttcggcaaccagaagctgcaggtgggcgagaagtacgtggtgttcgagaacgccgagagcgtggagaacctgatcgacaccgacaaggactacaacctggacaccaagcaggtggtgaagcacgaggacaagaacgacaaggcccagaccctggtggtggagaagccc

SEQ ID NO: 69 - protein, 170 aa, Homo sapiens
RMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 70 - protein, 154 aa, Homo sapiens
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 71 - protein, 122 aa, Homo sapiens
PANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCS

SEQ ID NO: 72 - protein, 205 aa, Homo sapiens
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 73 - protein, 282 aa, Thermotoga maritima
MGARASGSKSGSGSDSGSKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQ

SEQ ID NO: 74 - DNA, 510 bp, Homo sapiens
agaatgctgaaggccctgaacgaccagctgaacagagagctgtactccgcctacctgtacttcgctatggccgcctacttcgaggacctgggccttgagggattcgccaactggatgaaggctcaggccgaggaagagatcggccacgctctgagattctacaactacatctacgacagaaacggccgcgtggaactggacgagatccccaagcctcctaaagagtgggagagccctctgaaggctttcgaggctgcttacgagcacgagaagttcatcagcaagagcatctacgagctggccgctctggccgaagaggaaaaggactactctaccagagccttcctggaatggttcatcaacgaacaggtggaagaggaagccagcgtcaagaagatcctggacaagctgaagttcgccaaggacagccctcagatcctgttcatgctggacaaagagctgagcgccagggctcctaaactgcctggactgcttatgcaaggcggcgaa

SEQ ID NO: 75 - DNA, 462 bp, Homo sapiens
atgcagatctacgagggcaagctgaccgccgagggcctgaggttcggcatcgtggccagcaggttcaaccacgccctggtggacaggctggtggagggcgccatcgactgcatcgtgaggcacggcggcagggaggaggacatcaccctggtgagggtgcccggcagctgggagatccccgtggccgccggcgagctggccaggaaggaggacatcgacgccgtgatcgccatcggcgtgctgatcaggggcgccaccccccacttcgactacatcgccagcgaggtgagcaagggcctggccaacctgagcctggagctgaggaagcccatcaccttcggcgtgatcaccgccgacaccctggagcaggccatcgagagggccggcaccaagcacggcaacaagggctgggaggccgccctgagcgccatcgagatggccaacctgttcaagagcctgagg

SEQ ID NO: 76 - DNA, 366 bp, Homo sapiens
cccgccaacagcaccgtgctgagcttctgcgccttcgccgtggaccccgccaaggcctacaaggactacctggccagcggcggccagcccatcaccaactgcgtgaagatgctgtgcacccacaccggcaccggccaggccatcaccgtgacccccgaggccaacatggaccaggagagcttcggcggcgccagctgctgcctgtactgcaggtgccacatcgaccaccccaaccccaagggcttctgcgacctgaagggcaagtacgtgcagatccccaccacctgcgccaacgaccccgtgggcttcaccctgaggaacaccgtgtgcaccgtgtgcggcatgtggaagggctacggctgcagc

SEQ ID NO: 77 - DNA, 615 bp, Homo sapiens
atgaagatggaggagctgttcaagaagcacaagatcgtggccgtgctgagggccaacagcgtggaggaggccaagaagaaggccctggccgtgttcctgggcggcgtgcacctgatcgagatcaccttcaccgtgcccgacgccgacaccgtgatcaaggagctgagcttcctgaaggagatgggcgccatcatcggcgccggcaccgtgaccagcgtggagcaggccaggaaggccgtggagagcggcgccgagttcatcgtgagcccccacctggacgaggagatcagccagttcgccaaggagaagggcgtgttctacatgcccggcgtgatgacccccaccgagctggtgaaggccatgaagctgggccacaccatcctgaagctgttccccggcgaggtggtgggcccccagttcgtgaaggccatgaagggccccttccccaacgtgaagttcgtgcccaccggcggcgtgaacctggacaacgtgtgcgagtggttcaaggccggcgtgctggccgtgggcgtgggcagcgccctggtgaagggcacccccgtggaggtggccgagaaggccaaggccttcgtggagaagatcaggggctgcaccgag

SEQ ID NO: 78 - DNA, 846 bp, Thermotoga maritima
atgggcgccagggccagcggcagcaagagcggcagcggcagcgacagcggcagcaagatggaggagctgttcaagaagcacaagatcgtggccgtgctgagggccaacagcgtggaggaggccaagaagaaggccctggccgtgttcctgggcggcgtgcacctgatcgagatcaccttcaccgtgcccgacgccgacaccgtgatcaaggagctgagcttcctgaaggagatgggcgccatcatcggcgccggcaccgtgaccagcgtggagcagtgcaggaaggccgtggagagcggcgccgagttcatcgtgagcccccacctggacgaggagatcagccagttctgcaaggagaagggcgtgttctacatgcccggcgtgatgacccccaccgagctggtgaaggccatgaagctgggccacaccatcctgaagctgttccccggcgaggtggtgggcccccagttcgtgaaggccatgaagggccccttccccaacgtgaagttcgtgcccaccggcggcgtgaacctggacaacgtgtgcgagtggttcaaggccggcgtgctggccgtgggcgtgggcagcgccctggtgaagggcacccccgtggaggtggccgagaaggccaaggccttcgtggagaagatcaggggctgcaccgagcagaagctgatcagcgaggaggacctgcagagcaggcccgagcccaccgccccccccgaggagagcttcaggagcggcgtggagaccaccacccccccccagaagcaggagcccatcgacaaggagctgtaccccctgaccagcctgaggagcctgttcggcaacgaccccagcagccag

SEQ ID NO: 79 - protein, 19 aa, Homo sapiens
MTRLTVLALLAGLLASSRA

SEQ ID NO: 80 - protein, 18 aa, Homo sapiens
MKWVTFISLLFLFSSAYS

SEQ ID NO: 81 - protein, 19 aa, Homo sapiens
MKWVTFISLLFLFSSSSRA

SEQ ID NO: 82 - protein, 20 aa, Cricetulus griseus
MGSAALLLWVLLLWVPGSNG

SEQ ID NO: 83 - protein, 20 aa, Cricetulus griseus
MGSAALLLWVLLLWVPSSRA

SEQ ID NO: 84 - protein, 20 aa, Homo sapiens
MEAPAQLLFLLLLWLPSSRA

SEQ ID NO: 85 - protein, 154 aa, Artificial
  Vector
  MISC_FEATURE (1)..(154): pVax1_ SpyTag-AP205
AHIVMVDAYKPTKGSGTAGGGSGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 86 - protein, 198 aa, Artificial
  Vector
  MISC_FEATURE (1)..(198): pVAX1_ HBc-SpyTag
MDIDPYKEFGASVELLSFLPSDFFPSIRDLLDTASALYREALESPEHVSPHHTALRQAILCWGELMNLATWVGSNLEDPASRELVVSYVNVNMGLKLRQILWFHISCLTFGRETVLEYLVSFGVWIRTPTAYRPPNAPILSTLPETTVVGGGGGSPGGGTPSPGGGGSQSPGGGGSQSGESQCGSAHIVMVDAYKPTK

SEQ ID NO: 87 - protein, 360 aa, Artificial
  Vector
  MISC_FEATURE (1)..(360): pVAX1-SPYCEGFP
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 88 - DNA, 465 bp, Artificial
  Vector
  misc_feature (1)..(465): pVax1_ SpyTag-AP205
gctcacatcgtgatggtggacgcctacaagcccacaaaaggctctggaacagctggcggcggatctggctctgccaacaaacctatgcagcccatcaccagcaccgccaacaagatcgtttggagcgaccccaccagactgagcaccacattcagcgctagcctgctgagacagcgcgtgaaagtgggaatcgccgagctgaacaatgtgtccggccagtacgtgtccgtgtacaagaggcctgctcctaagcctgagggctgtgccgatgcctgtgtgatcatgcccaacgagaaccagagcatcagaaccgtgatcagcggcagcgccgagaacctggctacactgaaggctgagtgggagacacacaagagaaacgtggacaccctgttcgcctctggcaacgctggactgggcttcctggatcctacagccgctatcgtgtctagcgacaccacagcctga

SEQ ID NO: 89 - DNA, 597 bp, Artificial
  Vector
  misc_feature (1)..(597): pVAX1_ HBc-SpyTag
atggacatcgacccctacaaagaatttggcgccagcgtggaactgctgagcttcctgcctagcgacttcttcccttccatccgggacctgctggatacagccagcgcactgtatagagaggccctggaatctcccgagcacgtgtcacctcaccacacagctctgagacaggccatcctgtgttggggcgagctgatgaacctggccacatgggtcggaagcaacctggaagatcccgccagcagagaactggtggtgtcctacgtgaacgtgaacatgggcctgaagctgagacagatcctgtggttccacatcagctgcctgaccttcggcagagaaaccgtgctggaatacctggtgtccttcggcgtgtggatcagaacccctaccgcctacagacctcctaacgctcccatcctgagcaccctgcctgagacaacagttgttggcggaggcggaggatctcctggcggaggaacaccttctccaggtggtggtggatctcaaagtcctggtggtggcggttctcagagcggcgaatctcagtgtggctctgcccacatcgtgatggtggacgcctacaagcccaccaaatga

SEQ ID NO: 90 - DNA, 1082 bp, Artificial
  Vector
  misc_feature (1)..(1082): pVAX1-SPYCEGFP
gagccatggtggatacactgtctggactgtctagcgagcagggccagagcggcgacatgacaatcgaggaagatagcgccacacacatcaagttcagcaagcgcgacgaggacggcaaagaactggctggcgctaccatggaactgagagacagcagcggcaagaccatcagcacctggatctctgacggccaagtgaaggacttctatctgtaccccggcaagtacaccttcgtggaaacagccgctcctgacggatacgaggtggccacagccatcaccttcaccgtgaacgagcagggacaagtgacagtgaacggcaaggccacaaagggcgacgctcacattggcggctctggctccatggtgtccaagggcgaagaactgttcaccggcgtggtgcccattctggtggaactggatggggatgtgaacggccacaagttctccgtgtctggcgaaggcgaaggggatgccacatacggcaagctgaccctgaagttcatctgcaccaccggaaagctgcccgtgccttggcctacactggtcaccacactgacatacggcgtgcagtgcttcagcagataccccgaccatatgaagcagcacgacttcttcaagagcgccatgcctgagggctacgtgcaagagagaaccatcttctttaaggacgacggcaactacaagaccagggccgaagtgaagttcgagggcgacaccctggtcaacagaatcgagctgaagggcatcgacttcaaagaggatggcaacatcctgggccacaagctcgagtacaactacaacagccacaacgtgtacatcatggccgacaagcagaaaaacggcatcaaagtgaacttcaagatccggcacaacatcgaggacggaagcgtgcagctggccgatcactaccagcagaacacacctatcggcgacggacctgtgctgctgcctgataaccactacctgagcacacagagcgccctgagcaaggaccctaacgagaagagggaccacatggtgctgctggaatttgtgaccgccgctggcatcacactcggcatggacgagctgtacaaatga

SEQ ID NO: 91 - protein, 4 aa, Artificial
  C-terminal tag
  misc_feature (1)..(4): C-tag
EPEA

SEQ ID NO: 92 - protein, 183 aa, Hepatitis B virus
MDIDPYKEFGASVELLSFLPSDFFPSIRDLLDTASALYREALESPEHVSPHHTALRQAILCWGELMNLATWVGSNLEDPASRELVVSYVNVNMGLKLRQILWFHISCLTFGRETVLEYLVSFGVWIRTPTAYRPPNAPILSTLPETTVVGGGGGSPGGGTPSPGGGGSQSPGGGGSQSGESQC

SEQ ID NO: 93 - protein, 242 aa, Artificial
  Tandem hepatitis core protein construct
  misc_feature (1)..(242): Tandem hepatitis core protein
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVGGSGGSGGSGGSGGSMDIDPYKEFGATVELLSFLPSDFFPSVRDLLDTASALYREALESPEHCSPHHTALRQAILCWGELMTLATWVGNNLED

SEQ ID NO: 94 - protein, 239 aa, Artificial
  Enhanced green fluorescent protein
  misc_feature (1)..(239): eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 95 - protein, 6 aa, Artificial
  His-tag
  misc_feature (1)..(6): His purification tag
HHHHHH

SEQ ID NO: 96 - protein, 171 aa, Plasmodium falciparum
KVTVDTVCKRGFLIQMSGHLECKCENDLVLVNEETCEEKVLKCDEKTVNKPCGDFSKCIKIDGNPVSYACKCNLGYDMVNNVCIPNECKQVTCGNGKCILDTSNPVKTGVCSCNIGKVPNVQDQNKCSKDGETKCSLKCLKEQETCKAVDGIYKCDCKDGFIIDQESSICT

SEQ ID NO: 97 - protein, 254 aa, Natronobacterium gregoryi
KAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 98 - protein, 539 aa, Norovirus
KMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNDYDPTEEIPAPLGTPDFVGKIQGVLTQTTRTDGSTRGHKATVYTGSADFAPKLGRVQFETDTDRDFEANQNTKFTPVGVIQDGGTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 99 - protein, 30 aa, Homo sapiens
MKRGLCCVLLLCGAVFVSPSQEIHARFRRA

SEQ ID NO: 100 - protein, 18 aa, Homo sapiens
MAFLWLLSCWALLGTTFG

SEQ ID NO: 101 - protein, 17 aa, Homo sapiens
MLLLLLLLGLRLQLSLG